SEQUENCE LISTING (1391 sequences)

SEQ ID NO: 1 - protein, 10 aa, Artificial Sequence
  phosphopeptide or phosphopeptide variant
ALRSSPIMRK

SEQ ID NO: 2 - protein, 9 aa, Artificial Sequence
  phosphopeptide or phosphopeptide variant
ALYSGVHKK

SEQ ID NO: 3 - protein, 10 aa, Artificial Sequence
  phosphopeptide or phosphopeptide variant
DTVPLSPLKY

SEQ ID NO: 4 - protein, 10 aa, Artificial Sequence
  phosphopeptide or phosphopeptide variant
EMKKSPTSLK

SEQ ID NO: 5 - protein, 13 aa, Artificial Sequence
  phosphopeptide or phosphopeptide variant
FVSKVMIGSPKKV

SEQ ID NO: 6 - protein, 11 aa, Artificial Sequence
  phosphopeptide or phosphopeptide variant
KGISSSSLKEK

SEQ ID NO: 7 - protein, 10 aa, Artificial Sequence
  phosphopeptide or phosphopeptide variant
KIDSPTKVKK

SEQ ID NO: 8 - protein, 9 aa, Artificial Sequence
  phosphopeptide or phosphopeptide variant
KIFSKQQGK

SEQ ID NO: 9 - protein, 10 aa, Artificial Sequence
  phosphopeptide or phosphopeptide variant
KIRSSPREAK

SEQ ID NO: 10 - protein, 9 aa, Artificial Sequence
  phosphopeptide or phosphopeptide variant
KIRTSPTFR

SEQ ID NO: 11 - protein, 9 aa, Artificial Sequence
  phosphopeptide or phosphopeptide variant
KLFSPAHKK

SEQ ID NO: 12 - protein, 9 aa, Artificial Sequence
  phosphopeptide or phosphopeptide variant
KLRSPFLQK

SEQ ID NO: 13 - protein, 11 aa, Artificial Sequence
  phosphopeptide or phosphopeptide variant
KLSSPRGGMKK

SEQ ID NO: 14 - protein, 10 aa, Artificial Sequence
  phosphopeptide or phosphopeptide variant
KMPTTPVKAK

SEQ ID NO: 15 - protein, 9 aa, Artificial Sequence
  phosphopeptide or phosphopeptide variant
KRFSGTVRL

SEQ ID NO: 16 - protein, 9 aa, Artificial Sequence
  phosphopeptide or phosphopeptide variant
KRYSGNMEY

SEQ ID NO: 17 - protein, 11 aa, Artificial Sequence
  phosphopeptide or phosphopeptide variant
KSKSNPDFLKK

SEQ ID NO: 18 - protein, 9 aa, Artificial Sequence
  phosphopeptide or phosphopeptide variant
KSKTPLVAR

SEQ ID NO: 19 - protein, 10 aa, Artificial Sequence
  phosphopeptide or phosphopeptide variant
KSSSLGNLKK

SEQ ID NO: 20 - protein, 12 aa, Artificial Sequence
  phosphopeptide or phosphopeptide variant
KTLSPGKNGVVK

SEQ ID NO: 21 - protein, 10 aa, Artificial Sequence
  phosphopeptide or phosphopeptide variant
KTPTSPLKMK

SEQ ID NO: 22 - protein, 11 aa, Artificial Sequence
  phosphopeptide or phosphopeptide variant
KVHGSLARAGK

SEQ ID NO: 23 - protein, 9 aa, Artificial Sequence
  phosphopeptide or phosphopeptide variant
KVLTPIKEK

SEQ ID NO: 24 - protein, 10 aa, Artificial Sequence
  phosphopeptide or phosphopeptide variant
LLNKSSPVKK

SEQ ID NO: 25 - protein, 10 aa, Artificial Sequence
  phosphopeptide or phosphopeptide variant
MTRSPPRVSK

SEQ ID NO: 26 - protein, 9 aa, Artificial Sequence
  phosphopeptide or phosphopeptide variant
NYVERKNSL

SEQ ID NO: 27 - protein, 10 aa, Artificial Sequence
  phosphopeptide or phosphopeptide variant
QVFSPKKGQK

SEQ ID NO: 28 - protein, 10 aa, Artificial Sequence
  phosphopeptide or phosphopeptide variant
RAFSFSKTPK

SEQ ID NO: 29 - protein, 9 aa, Artificial Sequence
  phosphopeptide or phosphopeptide variant
RIYQYIQSR

SEQ ID NO: 30 - protein, 9 aa, Artificial Sequence
  phosphopeptide or phosphopeptide variant
RKLSVILIL

SEQ ID NO: 31 - protein, 9 aa, Artificial Sequence
  phosphopeptide or phosphopeptide variant
RLISPYKKK

SEQ ID NO: 32 - protein, 9 aa, Artificial Sequence
  phosphopeptide or phosphopeptide variant
RLKSPFRKK

SEQ ID NO: 33 - protein, 12 aa, Artificial Sequence
  phosphopeptide or phosphopeptide variant
RLLDRSPSRSAK

SEQ ID NO: 34 - protein, 9 aa, Artificial Sequence
  phosphopeptide or phosphopeptide variant
RLPSSTLKR

SEQ ID NO: 35 - protein, 10 aa, Artificial Sequence
  phosphopeptide or phosphopeptide variant
RLRSAGAAQK

SEQ ID NO: 36 - protein, 9 aa, Artificial Sequence
  phosphopeptide or phosphopeptide variant
RLSSPISKR

SEQ ID NO: 37 - protein, 9 aa, Artificial Sequence
  phosphopeptide or phosphopeptide variant
RLSSRYSQK

SEQ ID NO: 38 - protein, 9 aa, Artificial Sequence
  phosphopeptide or phosphopeptide variant
RMYSKSRDH

SEQ ID NO: 39 - protein, 11 aa, Artificial Sequence
  phosphopeptide or phosphopeptide variant
RSASSATQVHK

SEQ ID NO: 40 - protein, 9 aa, Artificial Sequence
  phosphopeptide or phosphopeptide variant
RSRRSPLLK

SEQ ID NO: 41 - protein, 10 aa, Artificial Sequence
  phosphopeptide or phosphopeptide variant
RSRSPPPVSK

SEQ ID NO: 42 - protein, 10 aa, Artificial Sequence
  phosphopeptide or phosphopeptide variant
RSRTSPITRR

SEQ ID NO: 43 - protein, 9 aa, Artificial Sequence
  phosphopeptide or phosphopeptide variant
RSSSLIRHK

SEQ ID NO: 44 - protein, 9 aa, Artificial Sequence
  phosphopeptide or phosphopeptide variant
RSVSLSMRK

SEQ ID NO: 45 - protein, 9 aa, Artificial Sequence
  phosphopeptide or phosphopeptide variant
RSYSGSRSR

SEQ ID NO: 46 - protein, 10 aa, Artificial Sequence
  phosphopeptide or phosphopeptide variant
RSYSPDHRQK

SEQ ID NO: 47 - protein, 9 aa, Artificial Sequence
  phosphopeptide or phosphopeptide variant
RSYSPERSK

SEQ ID NO: 48 - protein, 9 aa, Artificial Sequence
  phosphopeptide or phosphopeptide variant
RSYSPRNSR

SEQ ID NO: 49 - protein, 9 aa, Artificial Sequence
  phosphopeptide or phosphopeptide variant
RSYSYPRQK

SEQ ID NO: 50 - protein, 14 aa, Artificial Sequence
  phosphopeptide or phosphopeptide variant
RSYVTTSTRTYSLG

SEQ ID NO: 51 - protein, 10 aa, Artificial Sequence
  phosphopeptide or phosphopeptide variant
RTPSFLKKNK

SEQ ID NO: 52 - protein, 9 aa, Artificial Sequence
  phosphopeptide or phosphopeptide variant
RTYKSPLRH

SEQ ID NO: 53 - protein, 10 aa, Artificial Sequence
  phosphopeptide or phosphopeptide variant
RVASPTSGVK

SEQ ID NO: 54 - protein, 11 aa, Artificial Sequence
  phosphopeptide or phosphopeptide variant
RVASPTSGVKR

SEQ ID NO: 55 - protein, 10 aa, Artificial Sequence
  phosphopeptide or phosphopeptide variant
RVKLPSGSKK

SEQ ID NO: 56 - protein, 11 aa, Artificial Sequence
  phosphopeptide or phosphopeptide variant
RVKSPGSGHVK

SEQ ID NO: 57 - protein, 11 aa, Artificial Sequence
  phosphopeptide or phosphopeptide variant
RVKSPSPKSER

SEQ ID NO: 58 - protein, 11 aa, Artificial Sequence
  phosphopeptide or phosphopeptide variant
RVKTPTSQSYR

SEQ ID NO: 59 - protein, 9 aa, Artificial Sequence
  phosphopeptide or phosphopeptide variant
RVKTTPLRR

SEQ ID NO: 60 - protein, 10 aa, Artificial Sequence
  phosphopeptide or phosphopeptide variant
RVRQSPLATR

SEQ ID NO: 61 - protein, 9 aa, Artificial Sequence
  phosphopeptide or phosphopeptide variant
RVYSPYNHR

SEQ ID NO: 62 - protein, 9 aa, Artificial Sequence
  phosphopeptide or phosphopeptide variant
SLFSPRRNK

SEQ ID NO: 63 - protein, 11 aa, Artificial Sequence
  phosphopeptide or phosphopeptide variant
SLLNKSSPVKK

SEQ ID NO: 64 - protein, 9 aa, Artificial Sequence
  phosphopeptide or phosphopeptide variant
SLMSPGRRK

SEQ ID NO: 65 - protein, 9 aa, Artificial Sequence
  phosphopeptide or phosphopeptide variant
SVKSPVTVK

SEQ ID NO: 66 - protein, 9 aa, Artificial Sequence
  phosphopeptide or phosphopeptide variant
SVRRSVLMK

SEQ ID NO: 67 - protein, 9 aa, Artificial Sequence
  phosphopeptide or phosphopeptide variant
SVYSPVKKK

SEQ ID NO: 68 - protein, 12 aa, Artificial Sequence
  phosphopeptide or phosphopeptide variant
VSKVMIGSPKKV

SEQ ID NO: 69 - protein, 10 aa, Artificial Sequence
  phosphopeptide or phosphopeptide variant
VTQTPPYVKK

SEQ ID NO: 70 - protein, 11 aa, Artificial Sequence
  phosphopeptide or phosphopeptide variant
AEEEIGTPRKF

SEQ ID NO: 71 - protein, 8 aa, Artificial Sequence
  phosphopeptide or phosphopeptide variant
ESESLPRY

SEQ ID NO: 72 - protein, 10 aa, Artificial Sequence
  phosphopeptide or phosphopeptide variant
FSSSHEGFSY

SEQ ID NO: 73 - protein, 11 aa, Artificial Sequence
  phosphopeptide or phosphopeptide variant
GEESSDDGKKY

SEQ ID NO: 74 - protein, 9 aa, Artificial Sequence
  phosphopeptide or phosphopeptide variant
IIETPHKEI

SEQ ID NO: 75 - protein, 9 aa, Artificial Sequence
  phosphopeptide or phosphopeptide variant
ISFSAHTDY

SEQ ID NO: 76 - protein, 9 aa, Artificial Sequence
  phosphopeptide or phosphopeptide variant
ISSSMHSLY

SEQ ID NO: 77 - protein, 9 aa, Artificial Sequence
  phosphopeptide or phosphopeptide variant
ITQGTPLKY

SEQ ID NO: 78 - protein, 8 aa, Artificial Sequence
  phosphopeptide or phosphopeptide variant
IVTDRDPL

SEQ ID NO: 79 - protein, 9 aa, Artificial Sequence
  phosphopeptide or phosphopeptide variant
KSESRQERY

SEQ ID NO: 80 - protein, 10 aa, Artificial Sequence
  phosphopeptide or phosphopeptide variant
LLDPSRSYSY

SEQ ID NO: 81 - protein, 10 aa, Artificial Sequence
  phosphopeptide or phosphopeptide variant
LLDTPVKTQY

SEQ ID NO: 82 - protein, 10 aa, Artificial Sequence
  phosphopeptide or phosphopeptide variant
LSDSDTEAKL

SEQ ID NO: 83 - protein, 9 aa, Artificial Sequence
  phosphopeptide or phosphopeptide variant
MTDTYRLKY

SEQ ID NO: 84 - protein, 8 aa, Artificial Sequence
  phosphopeptide or phosphopeptide variant
NTDSPLRY

SEQ ID NO: 85 - protein, 10 aa, Artificial Sequence
  phosphopeptide or phosphopeptide variant
QLDSPQRALY

SEQ ID NO: 86 - protein, 9 aa, Artificial Sequence
  phosphopeptide or phosphopeptide variant
RGDSPKIDL

SEQ ID NO: 87 - protein, 9 aa, Artificial Sequence
  phosphopeptide or phosphopeptide variant
RSDSRAQAV

SEQ ID NO: 88 - protein, 11 aa, Artificial Sequence
  phosphopeptide or phosphopeptide variant
RSDSYVELSQY

SEQ ID NO: 89 - protein, 13 aa, Artificial Sequence
  phosphopeptide or phosphopeptide variant
RTEPSKSPGSLRY

SEQ ID NO: 90 - protein, 8 aa, Artificial Sequence
  phosphopeptide or phosphopeptide variant
SIDSPQKL

SEQ ID NO: 91 - protein, 10 aa, Artificial Sequence
  phosphopeptide or phosphopeptide variant
SLDSPSYVLY

SEQ ID NO: 92 - protein, 8 aa, Artificial Sequence
  phosphopeptide or phosphopeptide variant
SREKHSEI

SEQ ID NO: 93 - protein, 10 aa, Artificial Sequence
  phosphopeptide or phosphopeptide variant
SSDSPTNHFF

SEQ ID NO: 94 - protein, 9 aa, Artificial Sequence
  phosphopeptide or phosphopeptide variant
STDSETLRY

SEQ ID NO: 95 - protein, 9 aa, Artificial Sequence
  phosphopeptide or phosphopeptide variant
SVDISPTRL

SEQ ID NO: 96 - protein, 10 aa, Artificial Sequence
  phosphopeptide or phosphopeptide variant
TMASPGKDNY

SEQ ID NO: 97 - protein, 11 aa, Artificial Sequence
  phosphopeptide or phosphopeptide variant
TSETPDYLLKY

SEQ ID NO: 98 - protein, 11 aa, Artificial Sequence
  phosphopeptide or phosphopeptide variant
AEEEIGTPRKF

SEQ ID NO: 99 - protein, 10 aa, Artificial Sequence
  phosphopeptide or phosphopeptide variant
AENSPTRQQF

SEQ ID NO: 100 - protein, 11 aa, Artificial Sequence
  phosphopeptide or phosphopeptide variant
AESSPTAGKKL

SEQ ID NO: 101 - protein, 9 aa, Artificial Sequence
  phosphopeptide or phosphopeptide variant
ATAGPRLGW

SEQ ID NO: 102 - protein, 9 aa, Artificial Sequence
  phosphopeptide or phosphopeptide variant
DERLRINSL

SEQ ID NO: 103 - protein, 9 aa, Artificial Sequence
  phosphopeptide or phosphopeptide variant
DQFERIKTL

SEQ ID NO: 104 - protein, 9 aa, Artificial Sequence
  phosphopeptide or phosphopeptide variant
DQISHRASL

SEQ ID NO: 105 - protein, 10 aa, Artificial Sequence
  phosphopeptide or phosphopeptide variant
EESSDDGKKY

SEQ ID NO: 106 - protein, 10 aa, Artificial Sequence
  phosphopeptide or phosphopeptide variant
EESSDDGKKY

SEQ ID NO: 107 - protein, 11 aa, Artificial Sequence
  phosphopeptide or phosphopeptide variant
GEESSDDGKKY

SEQ ID NO: 108 - protein, 9 aa, Artificial Sequence
  phosphopeptide or phosphopeptide variant
KEMSPTRQL

SEQ ID NO: 109 - protein, 9 aa, Artificial Sequence
  phosphopeptide or phosphopeptide variant
RQKSPLFQF

SEQ ID NO: 110 - protein, 9 aa, Artificial Sequence
  phosphopeptide or phosphopeptide variant
SEGSLHRKY

SEQ ID NO: 111 - protein, 11 aa, Artificial Sequence
  phosphopeptide or phosphopeptide variant
ARFSPDDKYSR

SEQ ID NO: 112 - protein, 9 aa, Artificial Sequence
  phosphopeptide or phosphopeptide variant
FRYSGKTEY

SEQ ID NO: 113 - protein, 9 aa, Artificial Sequence
  phosphopeptide or phosphopeptide variant
GRKSPPPSF

SEQ ID NO: 114 - protein, 9 aa, Artificial Sequence
  phosphopeptide or phosphopeptide variant
GRLGSPHRR

SEQ ID NO: 115 - protein, 11 aa, Artificial Sequence
  phosphopeptide or phosphopeptide variant
GRLSPKASQVK

SEQ ID NO: 116 - protein, 10 aa, Artificial Sequence
  phosphopeptide or phosphopeptide variant
GRLSPVPVPR

SEQ ID NO: 117 - protein, 11 aa, Artificial Sequence
  phosphopeptide or phosphopeptide variant
GRSSTASLVKR

SEQ ID NO: 118 - protein, 11 aa, Artificial Sequence
  phosphopeptide or phosphopeptide variant
HRNSNPVIAEL

SEQ ID NO: 119 - protein, 9 aa, Artificial Sequence
  phosphopeptide or phosphopeptide variant
KRASFAKSV

SEQ ID NO: 120 - protein, 9 aa, Artificial Sequence
  phosphopeptide or phosphopeptide variant
KRASSPFRR

SEQ ID NO: 121 - protein, 9 aa, Artificial Sequence
  phosphopeptide or phosphopeptide variant
KRASVFVKL

SEQ ID NO: 122 - protein, 7 aa, Artificial Sequence
  phosphopeptide or phosphopeptide variant
KRFSFKK

SEQ ID NO: 123 - protein, 9 aa, Artificial Sequence
  phosphopeptide or phosphopeptide variant
KRFSFKKSF

SEQ ID NO: 124 - protein, 9 aa, Artificial Sequence
  phosphopeptide or phosphopeptide variant
KRFSGTVRL

SEQ ID NO: 125 - protein, 9 aa, Artificial Sequence
  phosphopeptide or phosphopeptide variant
KRLSPAPQL

SEQ ID NO: 126 - protein, 9 aa, Artificial Sequence
  phosphopeptide or phosphopeptide variant
KRLSVERIY

SEQ ID NO: 127 - protein, 8 aa, Artificial Sequence
  phosphopeptide or phosphopeptide variant
KRMSPKPL

SEQ ID NO: 128 - protein, 9 aa, Artificial Sequence
  phosphopeptide or phosphopeptide variant
KRYSGNMEY

SEQ ID NO: 129 - protein, 9 aa, Artificial Sequence
  phosphopeptide or phosphopeptide variant
MRLSRELQL

SEQ ID NO: 130 - protein, 10 aa, Artificial Sequence
  phosphopeptide or phosphopeptide variant
NRYTNRVVTL

SEQ ID NO: 131 - protein, 9 aa, Artificial Sequence
  phosphopeptide or phosphopeptide variant
RRFSPPRRM

SEQ ID NO: 132 - protein, 9 aa, Artificial Sequence
  phosphopeptide or phosphopeptide variant
RRFSRSPIR

SEQ ID NO: 133 - protein, 9 aa, Artificial Sequence
  phosphopeptide or phosphopeptide variant
RRISGVDRY

SEQ ID NO: 134 - protein, 9 aa, Artificial Sequence
  phosphopeptide or phosphopeptide variant
RRKSQLDSL

SEQ ID NO: 135 - protein, 9 aa, Artificial Sequence
  phosphopeptide or phosphopeptide variant
RRKSQVAEL

SEQ ID NO: 136 - protein, 9 aa, Artificial Sequence
  phosphopeptide or phosphopeptide variant
RRLSADIRL

SEQ ID NO: 137 - protein, 9 aa, Artificial Sequence
  phosphopeptide or phosphopeptide variant
RRLSFLVSY

SEQ ID NO: 138 - protein, 10 aa, Artificial Sequence
  phosphopeptide or phosphopeptide variant
RRLSGGSHSY

SEQ ID NO: 139 - protein, 10 aa, Artificial Sequence
  phosphopeptide or phosphopeptide variant
RRLSGPLHTL

SEQ ID NO: 140 - protein, 9 aa, Artificial Sequence
  phosphopeptide or phosphopeptide variant
RRMSLLSVV

SEQ ID NO: 141 - protein, 9 aa, Artificial Sequence
  phosphopeptide or phosphopeptide variant
RRNSSERTL

SEQ ID NO: 142 - protein, 9 aa, Artificial Sequence
  phosphopeptide or phosphopeptide variant
RRNSSIVGR

SEQ ID NO: 143 - protein, 10 aa, Artificial Sequence
  phosphopeptide or phosphopeptide variant
RRNSVFQQGM

SEQ ID NO: 144 - protein, 9 aa, Artificial Sequence
  phosphopeptide or phosphopeptide variant
RRPSLVHGY

SEQ ID NO: 145 - protein, 9 aa, Artificial Sequence
  phosphopeptide or phosphopeptide variant
RRPSVFERL

SEQ ID NO: 146 - protein, 9 aa, Artificial Sequence
  phosphopeptide or phosphopeptide variant
RRPSYRKIL

SEQ ID NO: 147 - protein, 9 aa, Artificial Sequence
  phosphopeptide or phosphopeptide variant
RRPSYTLGM

SEQ ID NO: 148 - protein, 9 aa, Artificial Sequence
  phosphopeptide or phosphopeptide variant
RRSSFLQVF

SEQ ID NO: 149 - protein, 9 aa, Artificial Sequence
  phosphopeptide or phosphopeptide variant
RRSSIGLRV

SEQ ID NO: 150 - protein, 9 aa, Artificial Sequence
  phosphopeptide or phosphopeptide variant
RRSSIQSTF

SEQ ID NO: 151 - protein, 9 aa, Artificial Sequence
  phosphopeptide or phosphopeptide variant
RRSSQSWSL

SEQ ID NO: 152 - protein, 9 aa, Artificial Sequence
  phosphopeptide or phosphopeptide variant
RRSSVDLGL

SEQ ID NO: 153 - protein, 9 aa, Artificial Sequence
  phosphopeptide or phosphopeptide variant
RRSSVDLGL

SEQ ID NO: 154 - protein, 9 aa, Artificial Sequence
  phosphopeptide or phosphopeptide variant
RRSSVKVEA

SEQ ID NO: 155 - protein, 9 aa, Artificial Sequence
  phosphopeptide or phosphopeptide variant
RRVVQRSSL

SEQ ID NO: 156 - protein, 9 aa, Artificial Sequence
  phosphopeptide or phosphopeptide variant
RRYSPPIQR

SEQ ID NO: 157 - protein, 8 aa, Artificial Sequence
  phosphopeptide or phosphopeptide variant
SRLTHLSL

SEQ ID NO: 158 - protein, 9 aa, Artificial Sequence
  phosphopeptide or phosphopeptide variant
SRMSPKAQR

SEQ ID NO: 159 - protein, 9 aa, Artificial Sequence
  phosphopeptide or phosphopeptide variant
SRTSPITRR

SEQ ID NO: 160 - protein, 10 aa, Artificial Sequence
  phosphopeptide or phosphopeptide variant
SRYSRSPYSR

SEQ ID NO: 161 - protein, 10 aa, Artificial Sequence
  phosphopeptide or phosphopeptide variant
SRYSRSPYSR

SEQ ID NO: 162 - protein, 10 aa, Artificial Sequence
  phosphopeptide or phosphopeptide variant
SRYSRSPYSR

SEQ ID NO: 163 - protein, 9 aa, Artificial Sequence
  phosphopeptide or phosphopeptide variant
RSISVGENL

SEQ ID NO: 164 - protein, 8 aa, Artificial Sequence
  phosphopeptide or phosphopeptide variant
RSRSPLEL

SEQ ID NO: 165 - protein, 9 aa, Artificial Sequence
  phosphopeptide or phosphopeptide variant
EPKRRSARL

SEQ ID NO: 166 - protein, 9 aa, Artificial Sequence
  phosphopeptide or phosphopeptide variant
EPRSPSHSM

SEQ ID NO: 167 - protein, 10 aa, Artificial Sequence
  phosphopeptide or phosphopeptide variant
FRRSPTKSSL

SEQ ID NO: 168 - protein, 12 aa, Artificial Sequence
  phosphopeptide or phosphopeptide variant
FRRSPTKSSLDY

SEQ ID NO: 169 - protein, 8 aa, Artificial Sequence
  phosphopeptide or phosphopeptide variant
FSISPVRL

SEQ ID NO: 170 - protein, 10 aa, Artificial Sequence
  phosphopeptide or phosphopeptide variant
GPRSASLLSL

SEQ ID NO: 171 - protein, 10 aa, Artificial Sequence
  phosphopeptide or phosphopeptide variant
GPRSASLLSL

SEQ ID NO: 172 - protein, 9 aa, Artificial Sequence
  phosphopeptide or phosphopeptide variant
HPRSPTPTL

SEQ ID NO: 173 - protein, 11 aa, Artificial Sequence
  phosphopeptide or phosphopeptide variant
KPASPKFIVTL

SEQ ID NO: 174 - protein, 9 aa, Artificial Sequence
  phosphopeptide or phosphopeptide variant
KPPHSPLVL

SEQ ID NO: 175 - protein, 10 aa, Artificial Sequence
  phosphopeptide or phosphopeptide variant
KPPSPEHQSL

SEQ ID NO: 176 - protein, 10 aa, Artificial Sequence
  phosphopeptide or phosphopeptide variant
KPPSPSPIEM

SEQ ID NO: 177 - protein, 9 aa, Artificial Sequence
  phosphopeptide or phosphopeptide variant
KPPTPGASF

SEQ ID NO: 178 - protein, 9 aa, Artificial Sequence
  phosphopeptide or phosphopeptide variant
KPPYRSHSL

SEQ ID NO: 179 - protein, 9 aa, Artificial Sequence
  phosphopeptide or phosphopeptide variant
KPQTRGKTF

SEQ ID NO: 180 - protein, 9 aa, Artificial Sequence
  phosphopeptide or phosphopeptide variant
KPRPPPLSP

SEQ ID NO: 181 - protein, 9 aa, Artificial Sequence
  phosphopeptide or phosphopeptide variant
KPRSPDHVL

SEQ ID NO: 182 - protein, 10 aa, Artificial Sequence
  phosphopeptide or phosphopeptide variant
KPRSPPRALV

SEQ ID NO: 183 - protein, 11 aa, Artificial Sequence
  phosphopeptide or phosphopeptide variant
KPRSPPRALVL

SEQ ID NO: 184 - protein, 9 aa, Artificial Sequence
  phosphopeptide or phosphopeptide variant
KPYSPLASL

SEQ ID NO: 185 - protein, 9 aa, Artificial Sequence
  phosphopeptide or phosphopeptide variant
LPIFSRLSI

SEQ ID NO: 186 - protein, 10 aa, Artificial Sequence
  phosphopeptide or phosphopeptide variant
MPRQPSATRL

SEQ ID NO: 187 - protein, 11 aa, Artificial Sequence
  phosphopeptide or phosphopeptide variant
QPRSPGPDYSL

SEQ ID NO: 188 - protein, 10 aa, Artificial Sequence
  phosphopeptide or phosphopeptide variant
QPRTPSPLVL

SEQ ID NO: 189 - protein, 10 aa, Artificial Sequence
  phosphopeptide or phosphopeptide variant
QPRTPSPLVL

SEQ ID NO: 190 - protein, 9 aa, Artificial Sequence
  phosphopeptide or phosphopeptide variant
RAPSPSSRM

SEQ ID NO: 191 - protein, 11 aa, Artificial Sequence
  phosphopeptide or phosphopeptide variant
RPASARAQPGL

SEQ ID NO: 192 - protein, 10 aa, Artificial Sequence
  phosphopeptide or phosphopeptide variant
RPATGGPGVA

SEQ ID NO: 193 - protein, 11 aa, Artificial Sequence
  phosphopeptide or phosphopeptide variant
RPDSRLGKTEL

SEQ ID NO: 194 - protein, 9 aa, Artificial Sequence
  phosphopeptide or phosphopeptide variant
RPHSPEKAF

SEQ ID NO: 195 - protein, 10 aa, Artificial Sequence
  phosphopeptide or phosphopeptide variant
RPITPPRNSA

SEQ ID NO: 196 - protein, 9 aa, Artificial Sequence
  phosphopeptide or phosphopeptide variant
RPISPGLSY

SEQ ID NO: 197 - protein, 9 aa, Artificial Sequence
  phosphopeptide or phosphopeptide variant
RPKLSSPAL

SEQ ID NO: 198 - protein, 9 aa, Artificial Sequence
  phosphopeptide or phosphopeptide variant
RPKPSSSPV

SEQ ID NO: 199 - protein, 10 aa, Artificial Sequence
  phosphopeptide or phosphopeptide variant
RPNSPSPTAL

SEQ ID NO: 200 - protein, 10 aa, Artificial Sequence
  phosphopeptide or phosphopeptide variant
RPPPPPDTPP

SEQ ID NO: 201 - protein, 9 aa, Artificial Sequence
  phosphopeptide or phosphopeptide variant
RPPSPGPVL

SEQ ID NO: 202 - protein, 10 aa, Artificial Sequence
  phosphopeptide or phosphopeptide variant
RPQRATSNVF

SEQ ID NO: 203 - protein, 11 aa, Artificial Sequence
  phosphopeptide or phosphopeptide variant
RPRANSGGVDL

SEQ ID NO: 204 - protein, 9 aa, Artificial Sequence
  phosphopeptide or phosphopeptide variant
RPRGSQSLL

SEQ ID NO: 205 - protein, 10 aa, Artificial Sequence
  phosphopeptide or phosphopeptide variant
RPRPHSAPSL

SEQ ID NO: 206 - protein, 8 aa, Artificial Sequence
  phosphopeptide or phosphopeptide variant
RPRSAVLL

SEQ ID NO: 207 - protein, 9 aa, Artificial Sequence
  phosphopeptide or phosphopeptide variant
RPRSLEVTI

SEQ ID NO: 208 - protein, 9 aa, Artificial Sequence
  phosphopeptide or phosphopeptide variant
RPRSPAARL

SEQ ID NO: 209 - protein, 10 aa, Artificial Sequence
  phosphopeptide or phosphopeptide variant
RPRSPRENSI

SEQ ID NO: 210 - protein, 9 aa, Artificial Sequence
  phosphopeptide or phosphopeptide variant
RPRSPSPIS

SEQ ID NO: 211 - protein, 8 aa, Artificial Sequence
  phosphopeptide or phosphopeptide variant
RPRSPTGP

SEQ ID NO: 212 - protein, 12 aa, Artificial Sequence
  phosphopeptide or phosphopeptide variant
RPRSPTGPSNSF

SEQ ID NO: 213 - protein, 10 aa, Artificial Sequence
  phosphopeptide or phosphopeptide variant
RPRPVSPSSL

SEQ ID NO: 214 - protein, 8 aa, Artificial Sequence
  phosphopeptide or phosphopeptide variant
RPSSLPDL

SEQ ID NO: 215 - protein, 9 aa, Artificial Sequence
  phosphopeptide or phosphopeptide variant
RPSRSSPGL

SEQ ID NO: 216 - protein, 9 aa, Artificial Sequence
  phosphopeptide or phosphopeptide variant
RPSSPALYF

SEQ ID NO: 217 - protein, 9 aa, Artificial Sequence
  phosphopeptide or phosphopeptide variant
RPTSRLNRL

SEQ ID NO: 218 - protein, 9 aa, Artificial Sequence
  phosphopeptide or phosphopeptide variant
RPVSPFQEL

SEQ ID NO: 219 - protein, 9 aa, Artificial Sequence
  phosphopeptide or phosphopeptide variant
RPVTPVSDL

SEQ ID NO: 220 - protein, 9 aa, Artificial Sequence
  phosphopeptide or phosphopeptide variant
RPWSPAVSA

SEQ ID NO: 221 - protein, 10 aa, Artificial Sequence
  phosphopeptide or phosphopeptide variant
RPYSPPFFSL

SEQ ID NO: 222 - protein, 9 aa, Artificial Sequence
  phosphopeptide or phosphopeptide variant
RRKSQVAEL

SEQ ID NO: 223 - protein, 12 aa, Artificial Sequence
  phosphopeptide or phosphopeptide variant
RRSSLDAEIDSL

SEQ ID NO: 224 - protein, 9 aa, Artificial Sequence
  phosphopeptide or phosphopeptide variant
RSESKDRKL

SEQ ID NO: 225 - protein, 9 aa, Artificial Sequence
  phosphopeptide or phosphopeptide variant
RSGSLERKV

SEQ ID NO: 226 - protein, 9 aa, Artificial Sequence
  phosphopeptide or phosphopeptide variant
RSLSPGGAA

SEQ ID NO: 227 - protein, 8 aa, Artificial Sequence
  phosphopeptide or phosphopeptide variant
RSLSPLLF

SEQ ID NO: 228 - protein, 10 aa, Artificial Sequence
  phosphopeptide or phosphopeptide variant
RTESDSGLKK

SEQ ID NO: 229 - protein, 9 aa, Artificial Sequence
  phosphopeptide or phosphopeptide variant
RVRSPTRSP

SEQ ID NO: 230 - protein, 11 aa, Artificial Sequence
  phosphopeptide or phosphopeptide variant
SPEKAGRRSSL

SEQ ID NO: 231 - protein, 10 aa, Artificial Sequence
  phosphopeptide or phosphopeptide variant
SPGLARKRSL

SEQ ID NO: 232 - protein, 9 aa, Artificial Sequence
  phosphopeptide or phosphopeptide variant
SPKSPGLKA

SEQ ID NO: 233 - protein, 9 aa, Artificial Sequence
  phosphopeptide or phosphopeptide variant
SPKSPTAAL

SEQ ID NO: 234 - protein, 9 aa, Artificial Sequence
  phosphopeptide or phosphopeptide variant
SPRSPGKPM

SEQ ID NO: 235 - protein, 10 aa, Artificial Sequence
  phosphopeptide or phosphopeptide variant
SPRRSRSISL

SEQ ID NO: 236 - protein, 11 aa, Artificial Sequence
  phosphopeptide or phosphopeptide variant
SPRTPVSPVKF

SEQ ID NO: 237 - protein, 11 aa, Artificial Sequence
  phosphopeptide or phosphopeptide variant
SPSSPSVRRQL

SEQ ID NO: 238 - protein, 13 aa, Artificial Sequence
  phosphopeptide or phosphopeptide variant
SPSTSRSGGSSRL

SEQ ID NO: 239 - protein, 8 aa, Artificial Sequence
  phosphopeptide or phosphopeptide variant
SPVVHQSL

SEQ ID NO: 240 - protein, 10 aa, Artificial Sequence
  phosphopeptide or phosphopeptide variant
TPAQPQRRSL

SEQ ID NO: 241 - protein, 11 aa, Artificial Sequence
  phosphopeptide or phosphopeptide variant
TPISPGRASGM

SEQ ID NO: 242 - protein, 9 aa, Artificial Sequence
  phosphopeptide or phosphopeptide variant
TPRSPPLGL

SEQ ID NO: 243 - protein, 10 aa, Artificial Sequence
  phosphopeptide or phosphopeptide variant
TPRSPPLGLI

SEQ ID NO: 244 - protein, 13 aa, Artificial Sequence
  phosphopeptide or phosphopeptide variant
VPRSPKHAHSSSL

SEQ ID NO: 245 - protein, 11 aa, Artificial Sequence
  phosphopeptide or phosphopeptide variant
YQRSFDEVEGV

SEQ ID NO: 246 - protein, 12 aa, Artificial Sequence
  phosphopeptide or phosphopeptide variant
YQRSFDEVEGVF

SEQ ID NO: 247 - protein, 10 aa, Artificial Sequence
  phosphopeptide or phosphopeptide variant
AMRSSPIMRK

SEQ ID NO: 248 - protein, 9 aa, Artificial Sequence
  phosphopeptide or phosphopeptide variant
AMYSGVHKK

SEQ ID NO: 249 - protein, 10 aa, Artificial Sequence
  phosphopeptide or phosphopeptide variant
DLVPLSPLKK

SEQ ID NO: 250 - protein, 10 aa, Artificial Sequence
  phosphopeptide or phosphopeptide variant
DMVPLSPLKK

SEQ ID NO: 251 - protein, 10 aa, Artificial Sequence
  phosphopeptide or phosphopeptide variant
ELKKSPTSLK

SEQ ID NO: 252 - protein, 11 aa, Artificial Sequence
  phosphopeptide or phosphopeptide variant
KLISSSSLKEK

SEQ ID NO: 253 - protein, 11 aa, Artificial Sequence
  phosphopeptide or phosphopeptide variant
KMISSSSLKEK

SEQ ID NO: 254 - protein, 10 aa, Artificial Sequence
  phosphopeptide or phosphopeptide variant
KLDSPTKVKK

SEQ ID NO: 255 - protein, 10 aa, Artificial Sequence
  phosphopeptide or phosphopeptide variant
KMDSPTKVKK

SEQ ID NO: 256 - protein, 9 aa, Artificial Sequence
  phosphopeptide or phosphopeptide variant
KLFSKQQGK

SEQ ID NO: 257 - protein, 9 aa, Artificial Sequence
  phosphopeptide or phosphopeptide variant
KMFSKQQGK

SEQ ID NO: 258 - protein, 10 aa, Artificial Sequence
  phosphopeptide or phosphopeptide variant
KLRSSPREAK

SEQ ID NO: 259 - protein, 10 aa, Artificial Sequence
  phosphopeptide or phosphopeptide variant
KMRSSPREAK

SEQ ID NO: 260 - protein, 9 aa, Artificial Sequence
  phosphopeptide or phosphopeptide variant
KLRTSPTFK

SEQ ID NO: 261 - protein, 9 aa, Artificial Sequence
  phosphopeptide or phosphopeptide variant
KMRTSPTFK

SEQ ID NO: 262 - protein, 9 aa, Artificial Sequence
  phosphopeptide or phosphopeptide variant
KMFSPAHKK

SEQ ID NO: 263 - protein, 9 aa, Artificial Sequence
  phosphopeptide or phosphopeptide variant
KMRSPFLQK

SEQ ID NO: 264 - protein, 11 aa, Artificial Sequence
  phosphopeptide or phosphopeptide variant
KMSSPRGGMKK

SEQ ID NO: 265 - protein, 10 aa, Artificial Sequence
  phosphopeptide or phosphopeptide variant
KLSSPRGGMK

SEQ ID NO: 266 - protein, 10 aa, Artificial Sequence
  phosphopeptide or phosphopeptide variant
KMSSPRGGMK

SEQ ID NO: 267 - protein, 10 aa, Artificial Sequence
  phosphopeptide or phosphopeptide variant
KLPTTPVKAK

SEQ ID NO: 268 - protein, 9 aa, Artificial Sequence
  phosphopeptide or phosphopeptide variant
KLFSGTVRK

SEQ ID NO: 269 - protein, 9 aa, Artificial Sequence
  phosphopeptide or phosphopeptide variant
KMFSGTVRK

SEQ ID NO: 270 - protein, 9 aa, Artificial Sequence
  phosphopeptide or phosphopeptide variant
KLYSGNMEK

SEQ ID NO: 271 - protein, 9 aa, Artificial Sequence
  phosphopeptide or phosphopeptide variant
KMYSGNMEK

SEQ ID NO: 272 - protein, 11 aa, Artificial Sequence
  phosphopeptide or phosphopeptide variant
KLKSNPDFLKK

SEQ ID NO: 273 - protein, 11 aa, Artificial Sequence
  phosphopeptide or phosphopeptide variant
KMKSNPDFLKK

SEQ ID NO: 274 - protein, 10 aa, Artificial Sequence
  phosphopeptide or phosphopeptide variant
KMKSNPDFLK

SEQ ID NO: 275 - protein, 10 aa, Artificial Sequence
  phosphopeptide or phosphopeptide variant
KLKSNPDFLK

SEQ ID NO: 276 - protein, 9 aa, Artificial Sequence
  phosphopeptide or phosphopeptide variant
KSKTPLVAK

SEQ ID NO: 277 - protein, 9 aa, Artificial Sequence
  phosphopeptide or phosphopeptide variant
KLKTPLVAR

SEQ ID NO: 278 - protein, 9 aa, Artificial Sequence
  phosphopeptide or phosphopeptide variant
KMKTPLVAR

SEQ ID NO: 279 - protein, 9 aa, Artificial Sequence
  phosphopeptide or phosphopeptide variant
KLKTPLVAK

SEQ ID NO: 280 - protein, 9 aa, Artificial Sequence
  phosphopeptide or phosphopeptide variant
KMKTPLVAK

SEQ ID NO: 281 - protein, 10 aa, Artificial Sequence
  phosphopeptide or phosphopeptide variant
KLSSLGNLKK

SEQ ID NO: 282 - protein, 10 aa, Artificial Sequence
  phosphopeptide or phosphopeptide variant
KMSSLGNLKK

SEQ ID NO: 283 - protein, 9 aa, Artificial Sequence
  phosphopeptide or phosphopeptide variant
KLSSLGNLK

SEQ ID NO: 284 - protein, 9 aa, Artificial Sequence
  phosphopeptide or phosphopeptide variant
KMSSLGNLK

SEQ ID NO: 285 - protein, 10 aa, Artificial Sequence
  phosphopeptide or phosphopeptide variant
KLPTSPLKMK

SEQ ID NO: 286 - protein, 10 aa, Artificial Sequence
  phosphopeptide or phosphopeptide variant
KMPTSPLKMK

SEQ ID NO: 287 - protein, 11 aa, Artificial Sequence
  phosphopeptide or phosphopeptide variant
KLHGSLARAGK

SEQ ID NO: 288 - protein, 11 aa, Artificial Sequence
  phosphopeptide or phosphopeptide variant
KMHGSLARAGK

SEQ ID NO: 289 - protein, 9 aa, Artificial Sequence
  phosphopeptide or phosphopeptide variant
KLLTPIKEK

SEQ ID NO: 290 - protein, 9 aa, Artificial Sequence
  phosphopeptide or phosphopeptide variant
KMLTPIKEK

SEQ ID NO: 291 - protein, 10 aa, Artificial Sequence
  phosphopeptide or phosphopeptide variant
LMNKSSPVKK

SEQ ID NO: 292 - protein, 9 aa, Artificial Sequence
  phosphopeptide or phosphopeptide variant
LLNKSSPVK

SEQ ID NO: 293 - protein, 9 aa, Artificial Sequence
  phosphopeptide or phosphopeptide variant
LMNKSSPVK

SEQ ID NO: 294 - protein, 10 aa, Artificial Sequence
  phosphopeptide or phosphopeptide variant
MLRSPPRVSK

SEQ ID NO: 295 - protein, 10 aa, Artificial Sequence
  phosphopeptide or phosphopeptide variant
MMRSPPRVSK

SEQ ID NO: 296 - protein, 9 aa, Artificial Sequence
  phosphopeptide or phosphopeptide variant
NLVERKNSL

SEQ ID NO: 297 - protein, 9 aa, Artificial Sequence
  phosphopeptide or phosphopeptide variant
NMVERKNSL

SEQ ID NO: 298 - protein, 9 aa, Artificial Sequence
  phosphopeptide or phosphopeptide variant
NLVERKNSK

SEQ ID NO: 299 - protein, 9 aa, Artificial Sequence
  phosphopeptide or phosphopeptide variant
NMVERKNSK

SEQ ID NO: 300 - protein, 9 aa, Artificial Sequence
  phosphopeptide or phosphopeptide variant
NYVERKNSK

SEQ ID NO: 301 - protein, 10 aa, Artificial Sequence
  phosphopeptide or phosphopeptide variant
QLFSPKKGQK

SEQ ID NO: 302 - protein, 10 aa, Artificial Sequence
  phosphopeptide or phosphopeptide variant
QMFSPKKGQK

SEQ ID NO: 303 - protein, 10 aa, Artificial Sequence
  phosphopeptide or phosphopeptide variant
RLFSFSKTPK

SEQ ID NO: 304 - protein, 10 aa, Artificial Sequence
  phosphopeptide or phosphopeptide variant
RMFSFSKTPK

SEQ ID NO: 305 - protein, 9 aa, Artificial Sequence
  phosphopeptide or phosphopeptide variant
RLYQYIQSR

SEQ ID NO: 306 - protein, 9 aa, Artificial Sequence
  phosphopeptide or phosphopeptide variant
RMYQYIQSR

SEQ ID NO: 307 - protein, 9 aa, Artificial Sequence
  phosphopeptide or phosphopeptide variant
RLYQYIQSK

SEQ ID NO: 308 - protein, 9 aa, Artificial Sequence
  phosphopeptide or phosphopeptide variant
RMYQYIQSK

SEQ ID NO: 309 - protein, 9 aa, Artificial Sequence
  phosphopeptide or phosphopeptide variant
RKLSVILIK

SEQ ID NO: 310 - protein, 9 aa, Artificial Sequence
  phosphopeptide or phosphopeptide variant
RLLSVILIK

SEQ ID NO: 311 - protein, 9 aa, Artificial Sequence
  phosphopeptide or phosphopeptide variant
RMLSVILIK

SEQ ID NO: 312 - protein, 9 aa, Artificial Sequence
  phosphopeptide or phosphopeptide variant
RMISPYKKK

SEQ ID NO: 313 - protein, 9 aa, Artificial Sequence
  phosphopeptide or phosphopeptide variant
RMKSPFRKK

SEQ ID NO: 314 - protein, 12 aa, Artificial Sequence
  phosphopeptide or phosphopeptide variant
RMLDRSPSRSAK

SEQ ID NO: 315 - protein, 9 aa, Artificial Sequence
  phosphopeptide or phosphopeptide variant
RLPSSTLKK

SEQ ID NO: 316 - protein, 9 aa, Artificial Sequence
  phosphopeptide or phosphopeptide variant
RMPSSTLKK

SEQ ID NO: 317 - protein, 9 aa, Artificial Sequence
  phosphopeptide or phosphopeptide variant
RMPSSTLKR

SEQ ID NO: 318 - protein, 10 aa, Artificial Sequence
  phosphopeptide or phosphopeptide variant
RMRSAGAAQK

SEQ ID NO: 319 - protein, 9 aa, Artificial Sequence
  phosphopeptide or phosphopeptide variant
RLSSPISKK

SEQ ID NO: 320 - protein, 9 aa, Artificial Sequence
  phosphopeptide or phosphopeptide variant
RMSSPISKR

SEQ ID NO: 321 - protein, 9 aa, Artificial Sequence
  phosphopeptide or phosphopeptide variant
RMSSPISKK

SEQ ID NO: 322 - protein, 9 aa, Artificial Sequence
  phosphopeptide or phosphopeptide variant
RMSSRYSQK

SEQ ID NO: 323 - protein, 9 aa, Artificial Sequence
  phosphopeptide or phosphopeptide variant
RMYSKSRDK

SEQ ID NO: 324 - protein, 9 aa, Artificial Sequence
  phosphopeptide or phosphopeptide variant
RMYSKSRDH

SEQ ID NO: 325 - protein, 9 aa, Artificial Sequence
  phosphopeptide or phosphopeptide variant
RLYSKSRDK

SEQ ID NO: 326 - protein, 11 aa, Artificial Sequence
  phosphopeptide or phosphopeptide variant
RLASSATQVHK

SEQ ID NO: 327 - protein, 11 aa, Artificial Sequence
  phosphopeptide or phosphopeptide variant
RMASSATQVHK

SEQ ID NO: 328 - protein, 9 aa, Artificial Sequence
  phosphopeptide or phosphopeptide variant
RLRRSPLLK

SEQ ID NO: 329 - protein, 9 aa, Artificial Sequence
  phosphopeptide or phosphopeptide variant
RMRRSPLLK

SEQ ID NO: 330 - protein, 10 aa, Artificial Sequence
  phosphopeptide or phosphopeptide variant
RLRSPPPVSK

SEQ ID NO: 331 - protein, 10 aa, Artificial Sequence
  phosphopeptide or phosphopeptide variant
RMRSPPPVSK

SEQ ID NO: 332 - protein, 10 aa, Artificial Sequence
  phosphopeptide or phosphopeptide variant
RLRTSPITRK

SEQ ID NO: 333 - protein, 10 aa, Artificial Sequence
  phosphopeptide or phosphopeptide variant
RMRTSPITRK

SEQ ID NO: 334 - protein, 10 aa, Artificial Sequence
  phosphopeptide or phosphopeptide variant
RMRTSPITRR

SEQ ID NO: 335 - protein, 10 aa, Artificial Sequence
  phosphopeptide or phosphopeptide variant
RMRTSPITRR

SEQ ID NO: 336 - protein, 9 aa, Artificial Sequence
  phosphopeptide or phosphopeptide variant
RLSSLIRHK

SEQ ID NO: 337 - protein, 9 aa, Artificial Sequence
  phosphopeptide or phosphopeptide variant
RMSSLIRHK

SEQ ID NO: 338 - protein, 9 aa, Artificial Sequence
  phosphopeptide or phosphopeptide variant
RLVSLSMRK

SEQ ID NO: 339 - protein, 9 aa, Artificial Sequence
  phosphopeptide or phosphopeptide variant
RMVSLSMRK

SEQ ID NO: 340 - protein, 9 aa, Artificial Sequence
  phosphopeptide or phosphopeptide variant
RLYSGSRSK

SEQ ID NO: 341 - protein, 9 aa, Artificial Sequence
  phosphopeptide or phosphopeptide variant
RMYSGSRSK

SEQ ID NO: 342 - protein, 9 aa, Artificial Sequence
  phosphopeptide or phosphopeptide variant
RMYSGSRSR

SEQ ID NO: 343 - protein, 9 aa, Artificial Sequence
  phosphopeptide or phosphopeptide variant
RLYSGSRSR

SEQ ID NO: 344 - protein, 10 aa, Artificial Sequence
  phosphopeptide or phosphopeptide variant
RLYSPDHRQK

SEQ ID NO: 345 - protein, 10 aa, Artificial Sequence
  phosphopeptide or phosphopeptide variant
RMYSPDHRQK

SEQ ID NO: 346 - protein, 9 aa, Artificial Sequence
  phosphopeptide or phosphopeptide variant
RLYSPERSK

SEQ ID NO: 347 - protein, 9 aa, Artificial Sequence
  phosphopeptide or phosphopeptide variant
RMYSPERSK

SEQ ID NO: 348 - protein, 9 aa, Artificial Sequence
  phosphopeptide or phosphopeptide variant
RLYSPRNSK

SEQ ID NO: 349 - protein, 9 aa, Artificial Sequence
  phosphopeptide or phosphopeptide variant
RMYSPRNSK

SEQ ID NO: 350 - protein, 9 aa, Artificial Sequence
  phosphopeptide or phosphopeptide variant
RLYSYPRQK

SEQ ID NO: 351 - protein, 9 aa, Artificial Sequence
  phosphopeptide or phosphopeptide variant
RMYSYPRQK

SEQ ID NO: 352 - protein, 14 aa, Artificial Sequence
  phosphopeptide or phosphopeptide variant
RLYVTTSTRTYSLK

SEQ ID NO: 353 - protein, 14 aa, Artificial Sequence
  phosphopeptide or phosphopeptide variant
RMYVTTSTRTYSLK

SEQ ID NO: 354 - protein, 14 aa, Artificial Sequence
  phosphopeptide or phosphopeptide variant
RLYVTTSTRTYSLG

SEQ ID NO: 355 - protein, 14 aa, Artificial Sequence
  phosphopeptide or phosphopeptide variant
RMYVTTSTRTYSLG

SEQ ID NO: 356 - protein, 10 aa, Artificial Sequence
  phosphopeptide or phosphopeptide variant
RLPSFLKKNK

SEQ ID NO: 357 - protein, 10 aa, Artificial Sequence
  phosphopeptide or phosphopeptide variant
RMPSFLKKNK

SEQ ID NO: 358 - protein, 9 aa, Artificial Sequence
  phosphopeptide or phosphopeptide variant
RLYKSPLRK

SEQ ID NO: 359 - protein, 9 aa, Artificial Sequence
  phosphopeptide or phosphopeptide variant
RMYKSPLRK

SEQ ID NO: 360 - protein, 9 aa, Artificial Sequence
  phosphopeptide or phosphopeptide variant
RLYKSPLRH

SEQ ID NO: 361 - protein, 9 aa, Artificial Sequence
  phosphopeptide or phosphopeptide variant
RMYKSPLRH

SEQ ID NO: 362 - protein, 9 aa, Artificial Sequence
  phosphopeptide or phosphopeptide variant
RTYKSPLRK

SEQ ID NO: 363 - protein, 10 aa, Artificial Sequence
  phosphopeptide or phosphopeptide variant
RLASPTSGVK

SEQ ID NO: 364 - protein, 10 aa, Artificial Sequence
  phosphopeptide or phosphopeptide variant
RMASPTSGVK

SEQ ID NO: 365 - protein, 11 aa, Artificial Sequence
  phosphopeptide or phosphopeptide variant
RLASPTSGVKK

SEQ ID NO: 366 - protein, 11 aa, Artificial Sequence
  phosphopeptide or phosphopeptide variant
RMASPTSGVKK

SEQ ID NO: 367 - protein, 11 aa, Artificial Sequence
  phosphopeptide or phosphopeptide variant
RLASPTSGVKR

SEQ ID NO: 368 - protein, 11 aa, Artificial Sequence
  phosphopeptide or phosphopeptide variant
RMASPTSGVKR

SEQ ID NO: 369 - protein, 11 aa, Artificial Sequence
  phosphopeptide or phosphopeptide variant
RVASPTSGVKK

SEQ ID NO: 370 - protein, 10 aa, Artificial Sequence
  phosphopeptide or phosphopeptide variant
RLKLPSGSKK

SEQ ID NO: 371 - protein, 10 aa, Artificial Sequence
  phosphopeptide or phosphopeptide variant
RMKLPSGSKK

SEQ ID NO: 372 - protein, 9 aa, Artificial Sequence
  phosphopeptide or phosphopeptide variant
RLKLPSGSK

SEQ ID NO: 373 - protein, 9 aa, Artificial Sequence
  phosphopeptide or phosphopeptide variant
RMKLPSGSK

SEQ ID NO: 374 - protein, 11 aa, Artificial Sequence
  phosphopeptide or phosphopeptide variant
RLKSPGSGHVK

SEQ ID NO: 375 - protein, 11 aa, Artificial Sequence
  phosphopeptide or phosphopeptide variant
RMKSPGSGHVK

SEQ ID NO: 376 - protein, 11 aa, Artificial Sequence
  phosphopeptide or phosphopeptide variant
RLKSPSPKSEK

SEQ ID NO: 377 - protein, 11 aa, Artificial Sequence
  phosphopeptide or phosphopeptide variant
RMKSPSPKSEK

SEQ ID NO: 378 - protein, 11 aa, Artificial Sequence
  phosphopeptide or phosphopeptide variant
RLKSPSPKSER

SEQ ID NO: 379 - protein, 11 aa, Artificial Sequence
  phosphopeptide or phosphopeptide variant
RMKSPSPKSER

SEQ ID NO: 380 - protein, 11 aa, Artificial Sequence
  phosphopeptide or phosphopeptide variant
RLKTPTSQSYK

SEQ ID NO: 381 - protein, 11 aa, Artificial Sequence
  phosphopeptide or phosphopeptide variant
RMKTPTSQSYK

SEQ ID NO: 382 - protein, 11 aa, Artificial Sequence
  phosphopeptide or phosphopeptide variant
RLKTPTSQSYR

SEQ ID NO: 383 - protein, 11 aa, Artificial Sequence
  phosphopeptide or phosphopeptide variant
RMKTPTSQSYR

SEQ ID NO: 384 - protein, 9 aa, Artificial Sequence
  phosphopeptide or phosphopeptide variant
RLKTTPLRK

SEQ ID NO: 385 - protein, 9 aa, Artificial Sequence
  phosphopeptide or phosphopeptide variant
RMKTTPLRK

SEQ ID NO: 386 - protein, 9 aa, Artificial Sequence
  phosphopeptide or phosphopeptide variant
RLKTTPLRR

SEQ ID NO: 387 - protein, 9 aa, Artificial Sequence
  phosphopeptide or phosphopeptide variant
RMKTTPLRR

SEQ ID NO: 388 - protein, 10 aa, Artificial Sequence
  phosphopeptide or phosphopeptide variant
RLRQSPLATK

SEQ ID NO: 389 - protein, 10 aa, Artificial Sequence
  phosphopeptide or phosphopeptide variant
RMRQSPLATK

SEQ ID NO: 390 - protein, 10 aa, Artificial Sequence
  phosphopeptide or phosphopeptide variant
RLRQSPLATR

SEQ ID NO: 391 - protein, 10 aa, Artificial Sequence
  phosphopeptide or phosphopeptide variant
RMRQSPLATR

SEQ ID NO: 392 - protein, 9 aa, Artificial Sequence
  phosphopeptide or phosphopeptide variant
RLYSPYNHK

SEQ ID NO: 393 - protein, 9 aa, Artificial Sequence
  phosphopeptide or phosphopeptide variant
RMYSPYNHK

SEQ ID NO: 394 - protein, 9 aa, Artificial Sequence
  phosphopeptide or phosphopeptide variant
RLYSPYNHR

SEQ ID NO: 395 - protein, 9 aa, Artificial Sequence
  phosphopeptide or phosphopeptide variant
RMYSPYNHR

SEQ ID NO: 396 - protein, 9 aa, Artificial Sequence
  phosphopeptide or phosphopeptide variant
SMFSPRRNK

SEQ ID NO: 397 - protein, 10 aa, Artificial Sequence
  phosphopeptide or phosphopeptide variant
SLLNKSSPVK

SEQ ID NO: 398 - protein, 10 aa, Artificial Sequence
  phosphopeptide or phosphopeptide variant
LLNKSSPVKK

SEQ ID NO: 399 - protein, 9 aa, Artificial Sequence
  phosphopeptide or phosphopeptide variant
LLNKSSPVK

SEQ ID NO: 400 - protein, 10 aa, Artificial Sequence
  phosphopeptide or phosphopeptide variant
SMLNKSSPVK

SEQ ID NO: 401 - protein, 10 aa, Artificial Sequence
  phosphopeptide or phosphopeptide variant
LMNKSSPVKK

SEQ ID NO: 402 - protein, 9 aa, Artificial Sequence
  phosphopeptide or phosphopeptide variant
LMNKSSPVK

SEQ ID NO: 403 - protein, 9 aa, Artificial Sequence
  phosphopeptide or phosphopeptide variant
SMMSPGRRK

SEQ ID NO: 404 - protein, 9 aa, Artificial Sequence
  phosphopeptide or phosphopeptide variant
SLKSPVTVK

SEQ ID NO: 405 - protein, 9 aa, Artificial Sequence
  phosphopeptide or phosphopeptide variant
SMKSPVTVK

SEQ ID NO: 406 - protein, 9 aa, Artificial Sequence
  phosphopeptide or phosphopeptide variant
SLRRSVLMK

SEQ ID NO: 407 - protein, 9 aa, Artificial Sequence
  phosphopeptide or phosphopeptide variant
SMRRSVLMK

SEQ ID NO: 408 - protein, 9 aa, Artificial Sequence
  phosphopeptide or phosphopeptide variant
SLYSPVKKK

SEQ ID NO: 409 - protein, 9 aa, Artificial Sequence
  phosphopeptide or phosphopeptide variant
SMYSPVKKK

SEQ ID NO: 410 - protein, 12 aa, Artificial Sequence
  phosphopeptide or phosphopeptide variant
VLKVMIGSPKKK

SEQ ID NO: 411 - protein, 11 aa, Artificial Sequence
  phosphopeptide or phosphopeptide variant
VLKVMIGSPKK

SEQ ID NO: 412 - protein, 10 aa, Artificial Sequence
  phosphopeptide or phosphopeptide variant
VLKVMIGSPK

SEQ ID NO: 413 - protein, 12 aa, Artificial Sequence
  phosphopeptide or phosphopeptide variant
VMKVMIGSPKKK

SEQ ID NO: 414 - protein, 11 aa, Artificial Sequence
  phosphopeptide or phosphopeptide variant
VMKVMIGSPKK

SEQ ID NO: 415 - protein, 10 aa, Artificial Sequence
  phosphopeptide or phosphopeptide variant
VMKVMIGSPK

SEQ ID NO: 416 - protein, 10 aa, Artificial Sequence
  phosphopeptide or phosphopeptide variant
VLQTPPYVKK

SEQ ID NO: 417 - protein, 10 aa, Artificial Sequence
  phosphopeptide or phosphopeptide variant
VMQTPPYVKK

SEQ ID NO: 418 - protein, 9 aa, Artificial Sequence
  phosphopeptide or phosphopeptide variant
VLQTPPYVK

SEQ ID NO: 419 - protein, 9 aa, Artificial Sequence
  phosphopeptide or phosphopeptide variant
VMQTPPYVK

SEQ ID NO: 420 - protein, 11 aa, Artificial Sequence
  phosphopeptide or phosphopeptide variant
ASEEIGTPRKY

SEQ ID NO: 421 - protein, 11 aa, Artificial Sequence
  phosphopeptide or phosphopeptide variant
ASDEIGTPRKY

SEQ ID NO: 422 - protein, 11 aa, Artificial Sequence
  phosphopeptide or phosphopeptide variant
ATDEIGTPRKY

SEQ ID NO: 423 - protein, 11 aa, Artificial Sequence
  phosphopeptide or phosphopeptide variant
ATEEIGTPRKY

SEQ ID NO: 424 - protein, 11 aa, Artificial Sequence
  phosphopeptide or phosphopeptide variant
ASEEIGTPRKF

SEQ ID NO: 425 - protein, 11 aa, Artificial Sequence
  phosphopeptide or phosphopeptide variant
ASDEIGTPRKF

SEQ ID NO: 426 - protein, 11 aa, Artificial Sequence
  phosphopeptide or phosphopeptide variant
ATDEIGTPRKF

SEQ ID NO: 427 - protein, 11 aa, Artificial Sequence
  phosphopeptide or phosphopeptide variant
ATEEIGTPRKF

SEQ ID NO: 428 - protein, 8 aa, Artificial Sequence
  phosphopeptide or phosphopeptide variant
ESDSLPRY

SEQ ID NO: 429 - protein, 8 aa, Artificial Sequence
  phosphopeptide or phosphopeptide variant
ETESLPRY

SEQ ID NO: 430 - protein, 8 aa, Artificial Sequence
  phosphopeptide or phosphopeptide variant
ETDSLPRY

SEQ ID NO: 431 - protein, 10 aa, Artificial Sequence
  phosphopeptide or phosphopeptide variant
FSDSHEGFSY

SEQ ID NO: 432 - protein, 10 aa, Artificial Sequence
  phosphopeptide or phosphopeptide variant
FTESHEGFSY

SEQ ID NO: 433 - protein, 10 aa, Artificial Sequence
  phosphopeptide or phosphopeptide variant
FSESHEGFSY

SEQ ID NO: 434 - protein, 10 aa, Artificial Sequence
  phosphopeptide or phosphopeptide variant
FTDSHEGFSY

SEQ ID NO: 435 - protein, 11 aa, Artificial Sequence
  phosphopeptide or phosphopeptide variant
GSESSDDGKKY

SEQ ID NO: 436 - protein, 11 aa, Artificial Sequence
  phosphopeptide or phosphopeptide variant
GSDSSDDGKKY

SEQ ID NO: 437 - protein, 11 aa, Artificial Sequence
  phosphopeptide or phosphopeptide variant
GTDSSDDGKKY

SEQ ID NO: 438 - protein, 11 aa, Artificial Sequence
  phosphopeptide or phosphopeptide variant
GTESSDDGKKY

SEQ ID NO: 439 - protein, 9 aa, Artificial Sequence
  phosphopeptide or phosphopeptide variant
IIETPHKEY

SEQ ID NO: 440 - protein, 9 aa, Artificial Sequence
  phosphopeptide or phosphopeptide variant
ISETPHKEI

SEQ ID NO: 441 - protein, 9 aa, Artificial Sequence
  phosphopeptide or phosphopeptide variant
ITETPHKEI

SEQ ID NO: 442 - protein, 9 aa, Artificial Sequence
  phosphopeptide or phosphopeptide variant
ISDTPHKEI

SEQ ID NO: 443 - protein, 9 aa, Artificial Sequence
  phosphopeptide or phosphopeptide variant
ITDTPHKEI

SEQ ID NO: 444 - protein, 9 aa, Artificial Sequence
  phosphopeptide or phosphopeptide variant
ISETPHKEY

SEQ ID NO: 445 - protein, 9 aa, Artificial Sequence
  phosphopeptide or phosphopeptide variant
ISDTPHKEY

SEQ ID NO: 446 - protein, 9 aa, Artificial Sequence
  phosphopeptide or phosphopeptide variant
ITDTPHKEY

SEQ ID NO: 447 - protein, 9 aa, Artificial Sequence
  phosphopeptide or phosphopeptide variant
ITETPHKEY

SEQ ID NO: 448 - protein, 9 aa, Artificial Sequence
  phosphopeptide or phosphopeptide variant
ISDSAHTDY

SEQ ID NO: 449 - protein, 9 aa, Artificial Sequence
  phosphopeptide or phosphopeptide variant
ISESAHTDY

SEQ ID NO: 450 - protein, 9 aa, Artificial Sequence
  phosphopeptide or phosphopeptide variant
ITESAHTDY

SEQ ID NO: 451 - protein, 9 aa, Artificial Sequence
  phosphopeptide or phosphopeptide variant
ITDSAHTDY

SEQ ID NO: 452 - protein, 9 aa, Artificial Sequence
  phosphopeptide or phosphopeptide variant
ISDSMHSLY

SEQ ID NO: 453 - protein, 9 aa, Artificial Sequence
  phosphopeptide or phosphopeptide variant
ISESMHSLY

SEQ ID NO: 454 - protein, 9 aa, Artificial Sequence
  phosphopeptide or phosphopeptide variant
ITESMHSLY

SEQ ID NO: 455 - protein, 9 aa, Artificial Sequence
  phosphopeptide or phosphopeptide variant
ITDSMHSLY

SEQ ID NO: 456 - protein, 9 aa, Artificial Sequence
  phosphopeptide or phosphopeptide variant
ITDGTPLKY

SEQ ID NO: 457 - protein, 9 aa, Artificial Sequence
  phosphopeptide or phosphopeptide variant
ITEGTPLKY

SEQ ID NO: 458 - protein, 9 aa, Artificial Sequence
  phosphopeptide or phosphopeptide variant
ISEGTPLKY

SEQ ID NO: 459 - protein, 9 aa, Artificial Sequence
  phosphopeptide or phosphopeptide variant
ISDGTPLKY

SEQ ID NO: 460 - protein, 8 aa, Artificial Sequence
  phosphopeptide or phosphopeptide variant
ISTDRDPL

SEQ ID NO: 461 - protein, 8 aa, Artificial Sequence
  phosphopeptide or phosphopeptide variant
ITTDRDPL

SEQ ID NO: 462 - protein, 8 aa, Artificial Sequence
  phosphopeptide or phosphopeptide variant
IVTDRDPY

SEQ ID NO: 463 - protein, 8 aa, Artificial Sequence
  phosphopeptide or phosphopeptide variant
ISTDRDPY

SEQ ID NO: 464 - protein, 8 aa, Artificial Sequence
  phosphopeptide or phosphopeptide variant
ITTDRDPY

SEQ ID NO: 465 - protein, 9 aa, Artificial Sequence
  phosphopeptide or phosphopeptide variant
KTDSRQERY

SEQ ID NO: 466 - protein, 9 aa, Artificial Sequence
  phosphopeptide or phosphopeptide variant
KSDSRQERY

SEQ ID NO: 467 - protein, 9 aa, Artificial Sequence
  phosphopeptide or phosphopeptide variant
KTESRQERY

SEQ ID NO: 468 - protein, 10 aa, Artificial Sequence
  phosphopeptide or phosphopeptide variant
LSDPSRSYSY

SEQ ID NO: 469 - protein, 10 aa, Artificial Sequence
  phosphopeptide or phosphopeptide variant
LSEPSRSYSY

SEQ ID NO: 470 - protein, 10 aa, Artificial Sequence
  phosphopeptide or phosphopeptide variant
LTEPSRSYSY

SEQ ID NO: 471 - protein, 10 aa, Artificial Sequence
  phosphopeptide or phosphopeptide variant
LTDPSRSYSY

SEQ ID NO: 472 - protein, 10 aa, Artificial Sequence
  phosphopeptide or phosphopeptide variant
LSDTPVKTQY

SEQ ID NO: 473 - protein, 10 aa, Artificial Sequence
  phosphopeptide or phosphopeptide variant
LSETPVKTQY

SEQ ID NO: 474 - protein, 10 aa, Artificial Sequence
  phosphopeptide or phosphopeptide variant
LTETPVKTQY

SEQ ID NO: 475 - protein, 10 aa, Artificial Sequence
  phosphopeptide or phosphopeptide variant
LTDTPVKTQY

SEQ ID NO: 476 - protein, 10 aa, Artificial Sequence
  phosphopeptide or phosphopeptide variant
LSESDTEAKL

SEQ ID NO: 477 - protein, 10 aa, Artificial Sequence
  phosphopeptide or phosphopeptide variant
LTESDTEAKL

SEQ ID NO: 478 - protein, 10 aa, Artificial Sequence
  phosphopeptide or phosphopeptide variant
LTDSDTEAKL

SEQ ID NO: 479 - protein, 10 aa, Artificial Sequence
  phosphopeptide or phosphopeptide variant
LSESDTEAKY

SEQ ID NO: 480 - protein, 10 aa, Artificial Sequence
  phosphopeptide or phosphopeptide variant
LSDSDTEAKY

SEQ ID NO: 481 - protein, 10 aa, Artificial Sequence
  phosphopeptide or phosphopeptide variant
LTDSDTEAKY

SEQ ID NO: 482 - protein, 10 aa, Artificial Sequence
  phosphopeptide or phosphopeptide variant
LTESDTEAKY

SEQ ID NO: 483 - protein, 9 aa, Artificial Sequence
  phosphopeptide or phosphopeptide variant
MSDTYRLKY

SEQ ID NO: 484 - protein, 9 aa, Artificial Sequence
  phosphopeptide or phosphopeptide variant
MSETYRLKY

SEQ ID NO: 485 - protein, 9 aa, Artificial Sequence
  phosphopeptide or phosphopeptide variant
MTETYRLKY

SEQ ID NO: 486 - protein, 8 aa, Artificial Sequence
  phosphopeptide or phosphopeptide variant
NSDSPLRY

SEQ ID NO: 487 - protein, 8 aa, Artificial Sequence
  phosphopeptide or phosphopeptide variant
NTESPLRY

SEQ ID NO: 488 - protein, 8 aa, Artificial Sequence
  phosphopeptide or phosphopeptide variant
NSESPLRY

SEQ ID NO: 489 - protein, 10 aa, Artificial Sequence
  phosphopeptide or phosphopeptide variant
QSDSPQRALY

SEQ ID NO: 490 - protein, 10 aa, Artificial Sequence
  phosphopeptide or phosphopeptide variant
QSESPQRALY

SEQ ID NO: 491 - protein, 10 aa, Artificial Sequence
  phosphopeptide or phosphopeptide variant
QTESPQRALY

SEQ ID NO: 492 - protein, 10 aa, Artificial Sequence
  phosphopeptide or phosphopeptide variant
QTDSPQRALY

SEQ ID NO: 493 - protein, 9 aa, Artificial Sequence
  phosphopeptide or phosphopeptide variant
RSDSPKIDL

SEQ ID NO: 494 - protein, 9 aa, Artificial Sequence
  phosphopeptide or phosphopeptide variant
RTDSPKIDL

SEQ ID NO: 495 - protein, 9 aa, Artificial Sequence
  phosphopeptide or phosphopeptide variant
RTESPKIDL

SEQ ID NO: 496 - protein, 9 aa, Artificial Sequence
  phosphopeptide or phosphopeptide variant
RSESPKIDL

SEQ ID NO: 497 - protein, 9 aa, Artificial Sequence
  phosphopeptide or phosphopeptide variant
RSDSPKIDY

SEQ ID NO: 498 - protein, 9 aa, Artificial Sequence
  phosphopeptide or phosphopeptide variant
RSESPKIDY

SEQ ID NO: 499 - protein, 9 aa, Artificial Sequence
  phosphopeptide or phosphopeptide variant
RTESPKIDY

SEQ ID NO: 500 - protein, 9 aa, Artificial Sequence
  phosphopeptide or phosphopeptide variant
RTDSPKIDY

SEQ ID NO: 501 - protein, 9 aa, Artificial Sequence
  phosphopeptide or phosphopeptide variant
RSESRAQAV

SEQ ID NO: 502 - protein, 9 aa, Artificial Sequence
  phosphopeptide or phosphopeptide variant
RTESRAQAV

SEQ ID NO: 503 - protein, 9 aa, Artificial Sequence
  phosphopeptide or phosphopeptide variant
RTDSRAQAV

SEQ ID NO: 504 - protein, 9 aa, Artificial Sequence
  phosphopeptide or phosphopeptide variant
RSESRAQAY

SEQ ID NO: 505 - protein, 9 aa, Artificial Sequence
  phosphopeptide or phosphopeptide variant
RSDSRAQAY

SEQ ID NO: 506 - protein, 9 aa, Artificial Sequence
  phosphopeptide or phosphopeptide variant
RTDSRAQAY

SEQ ID NO: 507 - protein, 9 aa, Artificial Sequence
  phosphopeptide or phosphopeptide variant
RTESRAQAY

SEQ ID NO: 508 - protein, 11 aa, Artificial Sequence
  phosphopeptide or phosphopeptide variant
RSESYVELSQY

SEQ ID NO: 509 - protein, 11 aa, Artificial Sequence
  phosphopeptide or phosphopeptide variant
RTESYVELSQY

SEQ ID NO: 510 - protein, 11 aa, Artificial Sequence
  phosphopeptide or phosphopeptide variant
RTDSYVELSQY

SEQ ID NO: 511 - protein, 13 aa, Artificial Sequence
  phosphopeptide or phosphopeptide variant
RSDPSKSPGSLRY

SEQ ID NO: 512 - protein, 13 aa, Artificial Sequence
  phosphopeptide or phosphopeptide variant
RTDPSKSPGSLRY

SEQ ID NO: 513 - protein, 13 aa, Artificial Sequence
  phosphopeptide or phosphopeptide variant
RSEPSKSPGSLRY

SEQ ID NO: 514 - protein, 8 aa, Artificial Sequence
  phosphopeptide or phosphopeptide variant
SSDSPQKL

SEQ ID NO: 515 - protein, 8 aa, Artificial Sequence
  phosphopeptide or phosphopeptide variant
SSESPQKL

SEQ ID NO: 516 - protein, 8 aa, Artificial Sequence
  phosphopeptide or phosphopeptide variant
SSESPQKY

SEQ ID NO: 517 - protein, 8 aa, Artificial Sequence
  phosphopeptide or phosphopeptide variant
SSDSPQKY

SEQ ID NO: 518 - protein, 8 aa, Artificial Sequence
  phosphopeptide or phosphopeptide variant
STESPQKY

SEQ ID NO: 519 - protein, 8 aa, Artificial Sequence
  phosphopeptide or phosphopeptide variant
STDSPQKY

SEQ ID NO: 520 - protein, 8 aa, Artificial Sequence
  phosphopeptide or phosphopeptide variant
SIDSPQKY

SEQ ID NO: 521 - protein, 10 aa, Artificial Sequence
  phosphopeptide or phosphopeptide variant
SSDSPSYVLY

SEQ ID NO: 522 - protein, 10 aa, Artificial Sequence
  phosphopeptide or phosphopeptide variant
SSESPSYVLY

SEQ ID NO: 523 - protein, 10 aa, Artificial Sequence
  phosphopeptide or phosphopeptide variant
STESPSYVLY

SEQ ID NO: 524 - protein, 10 aa, Artificial Sequence
  phosphopeptide or phosphopeptide variant
STDSPSYVLY

SEQ ID NO: 525 - protein, 10 aa, Artificial Sequence
  phosphopeptide or phosphopeptide variant
SLESPSYVLY

SEQ ID NO: 526 - protein, 8 aa, Artificial Sequence
  phosphopeptide or phosphopeptide variant
SSDKHSEY

SEQ ID NO: 527 - protein, 8 aa, Artificial Sequence
  phosphopeptide or phosphopeptide variant
SSEKHSEY

SEQ ID NO: 528 - protein, 8 aa, Artificial Sequence
  phosphopeptide or phosphopeptide variant
STEKHSEY

SEQ ID NO: 529 - protein, 8 aa, Artificial Sequence
  phosphopeptide or phosphopeptide variant
STDKHSEY

SEQ ID NO: 530 - protein, 8 aa, Artificial Sequence
  phosphopeptide or phosphopeptide variant
SRDKHSEY

SEQ ID NO: 531 - protein, 10 aa, Artificial Sequence
  phosphopeptide or phosphopeptide variant
SSESPTNHFY

SEQ ID NO: 532 - protein, 10 aa, Artificial Sequence
  phosphopeptide or phosphopeptide variant
STDSPTNHFY

SEQ ID NO: 533 - protein, 10 aa, Artificial Sequence
  phosphopeptide or phosphopeptide variant
STESPTNHFY

SEQ ID NO: 534 - protein, 9 aa, Artificial Sequence
  phosphopeptide or phosphopeptide variant
SSDSETLRY

SEQ ID NO: 535 - protein, 9 aa, Artificial Sequence
  phosphopeptide or phosphopeptide variant
STESETLRY

SEQ ID NO: 536 - protein, 9 aa, Artificial Sequence
  phosphopeptide or phosphopeptide variant
SSESETLRY

SEQ ID NO: 537 - protein, 9 aa, Artificial Sequence
  phosphopeptide or phosphopeptide variant
SSDISPTRL

SEQ ID NO: 538 - protein, 9 aa, Artificial Sequence
  phosphopeptide or phosphopeptide variant
STDISPTRL

SEQ ID NO: 539 - protein, 9 aa, Artificial Sequence
  phosphopeptide or phosphopeptide variant
STEISPTRL

SEQ ID NO: 540 - protein, 9 aa, Artificial Sequence
  phosphopeptide or phosphopeptide variant
STEISPTRL

SEQ ID NO: 541 - protein, 9 aa, Artificial Sequence
  phosphopeptide or phosphopeptide variant
SVDISPTRY

SEQ ID NO: 542 - protein, 9 aa, Artificial Sequence
  phosphopeptide or phosphopeptide variant
SSDISPTRY

SEQ ID NO: 543 - protein, 9 aa, Artificial Sequence
  phosphopeptide or phosphopeptide variant
SSEISPTRY

SEQ ID NO: 544 - protein, 9 aa, Artificial Sequence
  phosphopeptide or phosphopeptide variant
STDISPTRY

SEQ ID NO: 545 - protein, 9 aa, Artificial Sequence
  phosphopeptide or phosphopeptide variant
STEISPTRY

SEQ ID NO: 546 - protein, 10 aa, Artificial Sequence
  phosphopeptide or phosphopeptide variant
TMESPGKDNY

SEQ ID NO: 547 - protein, 10 aa, Artificial Sequence
  phosphopeptide or phosphopeptide variant
TMDSPGKDNY

SEQ ID NO: 548 - protein, 10 aa, Artificial Sequence
  phosphopeptide or phosphopeptide variant
TSDSPGKDNY

SEQ ID NO: 549 - protein, 10 aa, Artificial Sequence
  phosphopeptide or phosphopeptide variant
TSESPGKDNY

SEQ ID NO: 550 - protein, 10 aa, Artificial Sequence
  phosphopeptide or phosphopeptide variant
TTASPGKDNY

SEQ ID NO: 551 - protein, 10 aa, Artificial Sequence
  phosphopeptide or phosphopeptide variant
TSASPGKDNY

SEQ ID NO: 552 - protein, 10 aa, Artificial Sequence
  phosphopeptide or phosphopeptide variant
TTDSPGKDNY

SEQ ID NO: 553 - protein, 10 aa, Artificial Sequence
  phosphopeptide or phosphopeptide variant
TTESPGKDNY

SEQ ID NO: 554 - protein, 11 aa, Artificial Sequence
  phosphopeptide or phosphopeptide variant
TSDTPDYLLKY

SEQ ID NO: 555 - protein, 11 aa, Artificial Sequence
  phosphopeptide or phosphopeptide variant
TTDTPDYLLKY

SEQ ID NO: 556 - protein, 11 aa, Artificial Sequence
  phosphopeptide or phosphopeptide variant
TTETPDYLLKY

SEQ ID NO: 557 - protein, 11 aa, Artificial Sequence
  phosphopeptide or phosphopeptide variant
AEEEIGTPRKY

SEQ ID NO: 558 - protein, 11 aa, Artificial Sequence
  phosphopeptide or phosphopeptide variant
AEEEIGTPRKW

SEQ ID NO: 559 - protein, 10 aa, Artificial Sequence
  phosphopeptide or phosphopeptide variant
AENSPTRQQY

SEQ ID NO: 560 - protein, 10 aa, Artificial Sequence
  phosphopeptide or phosphopeptide variant
AENSPTRQQW

SEQ ID NO: 561 - protein, 11 aa, Artificial Sequence
  phosphopeptide or phosphopeptide variant
AESSPTAGKKF

SEQ ID NO: 562 - protein, 11 aa, Artificial Sequence
  phosphopeptide or phosphopeptide variant
AESSPTAGKKY

SEQ ID NO: 563 - protein, 11 aa, Artificial Sequence
  phosphopeptide or phosphopeptide variant
AESSPTAGKKW

SEQ ID NO: 564 - protein, 9 aa, Artificial Sequence
  phosphopeptide or phosphopeptide variant
ATAGPRLGF

SEQ ID NO: 565 - protein, 9 aa, Artificial Sequence
  phosphopeptide or phosphopeptide variant
ATAGPRLGY

SEQ ID NO: 566 - protein, 9 aa, Artificial Sequence
  phosphopeptide or phosphopeptide variant
DERLRINSF

SEQ ID NO: 567 - protein, 9 aa, Artificial Sequence
  phosphopeptide or phosphopeptide variant
DERLRINSY

SEQ ID NO: 568 - protein, 9 aa, Artificial Sequence
  phosphopeptide or phosphopeptide variant
DERLRINSW

SEQ ID NO: 569 - protein, 9 aa, Artificial Sequence
  phosphopeptide or phosphopeptide variant
DEFERIKTF

SEQ ID NO: 570 - protein, 9 aa, Artificial Sequence
  phosphopeptide or phosphopeptide variant
DEFERIKTY

SEQ ID NO: 571 - protein, 9 aa, Artificial Sequence
  phosphopeptide or phosphopeptide variant
DEFERIKTW

SEQ ID NO: 572 - protein, 9 aa, Artificial Sequence
  phosphopeptide or phosphopeptide variant
DEISHRASF

SEQ ID NO: 573 - protein, 9 aa, Artificial Sequence
  phosphopeptide or phosphopeptide variant
DEISHRASY

SEQ ID NO: 574 - protein, 9 aa, Artificial Sequence
  phosphopeptide or phosphopeptide variant
DEISHRASW

SEQ ID NO: 575 - protein, 10 aa, Artificial Sequence
  phosphopeptide or phosphopeptide variant
EESSDDGKKF

SEQ ID NO: 576 - protein, 10 aa, Artificial Sequence
  phosphopeptide or phosphopeptide variant
EESSDDGKKW

SEQ ID NO: 577 - protein, 10 aa, Artificial Sequence
  phosphopeptide or phosphopeptide variant
EESSDDGKKF

SEQ ID NO: 578 - protein, 10 aa, Artificial Sequence
  phosphopeptide or phosphopeptide variant
EESSDDGKKW

SEQ ID NO: 579 - protein, 11 aa, Artificial Sequence
  phosphopeptide or phosphopeptide variant
GEESSDDGKKF

SEQ ID NO: 580 - protein, 11 aa, Artificial Sequence
  phosphopeptide or phosphopeptide variant
GEESSDDGKKW

SEQ ID NO: 581 - protein, 9 aa, Artificial Sequence
  phosphopeptide or phosphopeptide variant
KEMSPTRQF

SEQ ID NO: 582 - protein, 9 aa, Artificial Sequence
  phosphopeptide or phosphopeptide variant
KEMSPTRQY

SEQ ID NO: 583 - protein, 9 aa, Artificial Sequence
  phosphopeptide or phosphopeptide variant
KEMSPTRQW

SEQ ID NO: 584 - protein, 9 aa, Artificial Sequence
  phosphopeptide or phosphopeptide variant
REKSPLFQF

SEQ ID NO: 585 - protein, 9 aa, Artificial Sequence
  phosphopeptide or phosphopeptide variant
REKSPLFQY

SEQ ID NO: 586 - protein, 9 aa, Artificial Sequence
  phosphopeptide or phosphopeptide variant
REKSPLFQW

SEQ ID NO: 587 - protein, 9 aa, Artificial Sequence
  phosphopeptide or phosphopeptide variant
SEGSLHRKF

SEQ ID NO: 588 - protein, 9 aa, Artificial Sequence
  phosphopeptide or phosphopeptide variant
SEGSLHRKW

SEQ ID NO: 589 - protein, 11 aa, Artificial Sequence
  phosphopeptide or phosphopeptide variant
ARFSPDDKYSL

SEQ ID NO: 590 - protein, 11 aa, Artificial Sequence
  phosphopeptide or phosphopeptide variant
ARFSPDDKYSF

SEQ ID NO: 591 - protein, 11 aa, Artificial Sequence
  phosphopeptide or phosphopeptide variant
ARFSPDDKYSM

SEQ ID NO: 592 - protein, 11 aa, Artificial Sequence
  phosphopeptide or phosphopeptide variant
ARFSPDDKYSK

SEQ ID NO: 593 - protein, 11 aa, Artificial Sequence
  phosphopeptide or phosphopeptide variant
RRFSPDDKYSR

SEQ ID NO: 594 - protein, 11 aa, Artificial Sequence
  phosphopeptide or phosphopeptide variant
RRFSPDDKYSL

SEQ ID NO: 595 - protein, 11 aa, Artificial Sequence
  phosphopeptide or phosphopeptide variant
RRFSPDDKYSF

SEQ ID NO: 596 - protein, 11 aa, Artificial Sequence
  phosphopeptide or phosphopeptide variant
RRFSPDDKYSK

SEQ ID NO: 597 - protein, 11 aa, Artificial Sequence
  phosphopeptide or phosphopeptide variant
RRFSPDDKYSM

SEQ ID NO: 598 - protein, 9 aa, Artificial Sequence
  phosphopeptide or phosphopeptide variant
FRYSGKTEL

SEQ ID NO: 599 - protein, 9 aa, Artificial Sequence
  phosphopeptide or phosphopeptide variant
FRYSGKTEF

SEQ ID NO: 600 - protein, 9 aa, Artificial Sequence
  phosphopeptide or phosphopeptide variant
FRYSGKTEK

SEQ ID NO: 601 - protein, 9 aa, Artificial Sequence
  phosphopeptide or phosphopeptide variant
FRYSGKTER

SEQ ID NO: 602 - protein, 9 aa, Artificial Sequence
  phosphopeptide or phosphopeptide variant
FRYSGKTEM

SEQ ID NO: 603 - protein, 9 aa, Artificial Sequence
  phosphopeptide or phosphopeptide variant
RRYSGKTEY

SEQ ID NO: 604 - protein, 9 aa, Artificial Sequence
  phosphopeptide or phosphopeptide variant
RRYSGKTEL

SEQ ID NO: 605 - protein, 9 aa, Artificial Sequence
  phosphopeptide or phosphopeptide variant
RRYSGKTEF

SEQ ID NO: 606 - protein, 9 aa, Artificial Sequence
  phosphopeptide or phosphopeptide variant
RRYSGKTER

SEQ ID NO: 607 - protein, 9 aa, Artificial Sequence
  phosphopeptide or phosphopeptide variant
RRYSGKTEK

SEQ ID NO: 608 - protein, 9 aa, Artificial Sequence
  phosphopeptide or phosphopeptide variant
GRKSPPPSL

SEQ ID NO: 609 - protein, 9 aa, Artificial Sequence
  phosphopeptide or phosphopeptide variant
GRKSPPPSR

SEQ ID NO: 610 - protein, 9 aa, Artificial Sequence
  phosphopeptide or phosphopeptide variant
GRKSPPPSK

SEQ ID NO: 611 - protein, 9 aa, Artificial Sequence
  phosphopeptide or phosphopeptide variant
GRKSPPPSM

SEQ ID NO: 612 - protein, 9 aa, Artificial Sequence
  phosphopeptide or phosphopeptide variant
RRKSPPPSF

SEQ ID NO: 613 - protein, 9 aa, Artificial Sequence
  phosphopeptide or phosphopeptide variant
RRKSPPPSL

SEQ ID NO: 614 - protein, 9 aa, Artificial Sequence
  phosphopeptide or phosphopeptide variant
RRKSPPPSR

SEQ ID NO: 615 - protein, 9 aa, Artificial Sequence
  phosphopeptide or phosphopeptide variant
RRKSPPPSM

SEQ ID NO: 616 - protein, 9 aa, Artificial Sequence
  phosphopeptide or phosphopeptide variant
RRKSPPPSK

SEQ ID NO: 617 - protein, 9 aa, Artificial Sequence
  phosphopeptide or phosphopeptide variant
GRLGSPHRL

SEQ ID NO: 618 - protein, 9 aa, Artificial Sequence
  phosphopeptide or phosphopeptide variant
GRLGSPHRF

SEQ ID NO: 619 - protein, 9 aa, Artificial Sequence
  phosphopeptide or phosphopeptide variant
GRLGSPHRK

SEQ ID NO: 620 - protein, 9 aa, Artificial Sequence
  phosphopeptide or phosphopeptide variant
GRLGSPHRM

SEQ ID NO: 621 - protein, 9 aa, Artificial Sequence
  phosphopeptide or phosphopeptide variant
RRLGSPHRR

SEQ ID NO: 622 - protein, 9 aa, Artificial Sequence
  phosphopeptide or phosphopeptide variant
RRLGSPHRL

SEQ ID NO: 623 - protein, 9 aa, Artificial Sequence
  phosphopeptide or phosphopeptide variant
RRLGSPHRF

SEQ ID NO: 624 - protein, 9 aa, Artificial Sequence
  phosphopeptide or phosphopeptide variant
RRLGSPHRK

SEQ ID NO: 625 - protein, 9 aa, Artificial Sequence
  phosphopeptide or phosphopeptide variant
RRLGSPHRM

SEQ ID NO: 626 - protein, 11 aa, Artificial Sequence
  phosphopeptide or phosphopeptide variant
GRLSPKASQVL

SEQ ID NO: 627 - protein, 11 aa, Artificial Sequence
  phosphopeptide or phosphopeptide variant
GRLSPKASQVF

SEQ ID NO: 628 - protein, 11 aa, Artificial Sequence
  phosphopeptide or phosphopeptide variant
GRLSPKASQVR

SEQ ID NO: 629 - protein, 11 aa, Artificial Sequence
  phosphopeptide or phosphopeptide variant
GRLSPKASQVM

SEQ ID NO: 630 - protein, 11 aa, Artificial Sequence
  phosphopeptide or phosphopeptide variant
RRLSPKASQVF

SEQ ID NO: 631 - protein, 11 aa, Artificial Sequence
  phosphopeptide or phosphopeptide variant
RRLSPKASQVR

SEQ ID NO: 632 - protein, 11 aa, Artificial Sequence
  phosphopeptide or phosphopeptide variant
RRLSPKASQVL

SEQ ID NO: 633 - protein, 11 aa, Artificial Sequence
  phosphopeptide or phosphopeptide variant
RRLSPKASQVK

SEQ ID NO: 634 - protein, 11 aa, Artificial Sequence
  phosphopeptide or phosphopeptide variant
RRLSPKASQVM

SEQ ID NO: 635 - protein, 10 aa, Artificial Sequence
  phosphopeptide or phosphopeptide variant
GRLSPVPVPL

SEQ ID NO: 636 - protein, 10 aa, Artificial Sequence
  phosphopeptide or phosphopeptide variant
GRLSPVPVPF

SEQ ID NO: 637 - protein, 10 aa, Artificial Sequence
  phosphopeptide or phosphopeptide variant
GRLSPVPVPM

SEQ ID NO: 638 - protein, 10 aa, Artificial Sequence
  phosphopeptide or phosphopeptide variant
GRLSPVPVPK

SEQ ID NO: 639 - protein, 10 aa, Artificial Sequence
  phosphopeptide or phosphopeptide variant
RRLSPVPVPR

SEQ ID NO: 640 - protein, 10 aa, Artificial Sequence
  phosphopeptide or phosphopeptide variant
RRLSPVPVPL

SEQ ID NO: 641 - protein, 10 aa, Artificial Sequence
  phosphopeptide or phosphopeptide variant
RRLSPVPVPF

SEQ ID NO: 642 - protein, 10 aa, Artificial Sequence
  phosphopeptide or phosphopeptide variant
RRLSPVPVPK

SEQ ID NO: 643 - protein, 10 aa, Artificial Sequence
  phosphopeptide or phosphopeptide variant
RRLSPVPVPM

SEQ ID NO: 644 - protein, 11 aa, Artificial Sequence
  phosphopeptide or phosphopeptide variant
GRSSTASLVKL

SEQ ID NO: 645 - protein, 11 aa, Artificial Sequence
  phosphopeptide or phosphopeptide variant
GRSSTASLVKF

SEQ ID NO: 646 - protein, 11 aa, Artificial Sequence
  phosphopeptide or phosphopeptide variant
GRSSTASLVKK

SEQ ID NO: 647 - protein, 11 aa, Artificial Sequence
  phosphopeptide or phosphopeptide variant
GRSSTASLVKM

SEQ ID NO: 648 - protein, 11 aa, Artificial Sequence
  phosphopeptide or phosphopeptide variant
RRSSTASLVKR

SEQ ID NO: 649 - protein, 11 aa, Artificial Sequence
  phosphopeptide or phosphopeptide variant
RRSSTASLVKL

SEQ ID NO: 650 - protein, 11 aa, Artificial Sequence
  phosphopeptide or phosphopeptide variant
RRSSTASLVKF

SEQ ID NO: 651 - protein, 11 aa, Artificial Sequence
  phosphopeptide or phosphopeptide variant
RRSSTASLVKM

SEQ ID NO: 652 - protein, 11 aa, Artificial Sequence
  phosphopeptide or phosphopeptide variant
RRSSTASLVKK

SEQ ID NO: 653 - protein, 11 aa, Artificial Sequence
  phosphopeptide or phosphopeptide variant
HRNSNPVIAEF

SEQ ID NO: 654 - protein, 11 aa, Artificial Sequence
  phosphopeptide or phosphopeptide variant
HRNSNPVIAEK

SEQ ID NO: 655 - protein, 11 aa, Artificial Sequence
  phosphopeptide or phosphopeptide variant
HRNSNPVIAER

SEQ ID NO: 656 - protein, 11 aa, Artificial Sequence
  phosphopeptide or phosphopeptide variant
HRNSNPVIAEM

SEQ ID NO: 657 - protein, 11 aa, Artificial Sequence
  phosphopeptide or phosphopeptide variant
RRNSNPVIAEL

SEQ ID NO: 658 - protein, 11 aa, Artificial Sequence
  phosphopeptide or phosphopeptide variant
RRNSNPVIAEF

SEQ ID NO: 659 - protein, 11 aa, Artificial Sequence
  phosphopeptide or phosphopeptide variant
RRNSNPVIAEK

SEQ ID NO: 660 - protein, 11 aa, Artificial Sequence
  phosphopeptide or phosphopeptide variant
RRNSNPVIAER

SEQ ID NO: 661 - protein, 11 aa, Artificial Sequence
  phosphopeptide or phosphopeptide variant
RRNSNPVIAEM

SEQ ID NO: 662 - protein, 9 aa, Artificial Sequence
  phosphopeptide or phosphopeptide variant
KRASFAKSL

SEQ ID NO: 663 - protein, 9 aa, Artificial Sequence
  phosphopeptide or phosphopeptide variant
KRASFAKSF

SEQ ID NO: 664 - protein, 9 aa, Artificial Sequence
  phosphopeptide or phosphopeptide variant
KRASFAKSK

SEQ ID NO: 665 - protein, 9 aa, Artificial Sequence
  phosphopeptide or phosphopeptide variant
KRASFAKSM

SEQ ID NO: 666 - protein, 9 aa, Artificial Sequence
  phosphopeptide or phosphopeptide variant
KRASFAKSR

SEQ ID NO: 667 - protein, 9 aa, Artificial Sequence
  phosphopeptide or phosphopeptide variant
RRASFAKSL

SEQ ID NO: 668 - protein, 9 aa, Artificial Sequence
  phosphopeptide or phosphopeptide variant
RRASFAKSF

SEQ ID NO: 669 - protein, 9 aa, Artificial Sequence
  phosphopeptide or phosphopeptide variant
RRASFAKSK

SEQ ID NO: 670 - protein, 9 aa, Artificial Sequence
  phosphopeptide or phosphopeptide variant
RRASFAKSM

SEQ ID NO: 671 - protein, 9 aa, Artificial Sequence
  phosphopeptide or phosphopeptide variant
RRASFAKSR

SEQ ID NO: 672 - protein, 9 aa, Artificial Sequence
  phosphopeptide or phosphopeptide variant
KRASSPFRL

SEQ ID NO: 673 - protein, 9 aa, Artificial Sequence
  phosphopeptide or phosphopeptide variant
KRASSPFRF

SEQ ID NO: 674 - protein, 9 aa, Artificial Sequence
  phosphopeptide or phosphopeptide variant
KRASSPFRK

SEQ ID NO: 675 - protein, 9 aa, Artificial Sequence
  phosphopeptide or phosphopeptide variant
KRASSPFRM

SEQ ID NO: 676 - protein, 9 aa, Artificial Sequence
  phosphopeptide or phosphopeptide variant
RRASSPFRR

SEQ ID NO: 677 - protein, 9 aa, Artificial Sequence
  phosphopeptide or phosphopeptide variant
RRASSPFRL

SEQ ID NO: 678 - protein, 9 aa, Artificial Sequence
  phosphopeptide or phosphopeptide variant
RRASSPFRF

SEQ ID NO: 679 - protein, 9 aa, Artificial Sequence
  phosphopeptide or phosphopeptide variant
RRASSPFRM

SEQ ID NO: 680 - protein, 9 aa, Artificial Sequence
  phosphopeptide or phosphopeptide variant
RRASSPFRK

SEQ ID NO: 681 - protein, 9 aa, Artificial Sequence
  phosphopeptide or phosphopeptide variant
KRASVFVKF

SEQ ID NO: 682 - protein, 9 aa, Artificial Sequence
  phosphopeptide or phosphopeptide variant
KRASVFVKK

SEQ ID NO: 683 - protein, 9 aa, Artificial Sequence
  phosphopeptide or phosphopeptide variant
KRASVFVKR

SEQ ID NO: 684 - protein, 9 aa, Artificial Sequence
  phosphopeptide or phosphopeptide variant
KRASVFVKM

SEQ ID NO: 685 - protein, 9 aa, Artificial Sequence
  phosphopeptide or phosphopeptide variant
RRASVFVKL

SEQ ID NO: 686 - protein, 9 aa, Artificial Sequence
  phosphopeptide or phosphopeptide variant
RRASVFVKF

SEQ ID NO: 687 - protein, 9 aa, Artificial Sequence
  phosphopeptide or phosphopeptide variant
RRASVFVKK

SEQ ID NO: 688 - protein, 9 aa, Artificial Sequence
  phosphopeptide or phosphopeptide variant
RRASVFVKR

SEQ ID NO: 689 - protein, 9 aa, Artificial Sequence
  phosphopeptide or phosphopeptide variant
RRASVFVKM

SEQ ID NO: 690 - protein, 7 aa, Artificial Sequence
  phosphopeptide or phosphopeptide variant
KRFSFKL

SEQ ID NO: 691 - protein, 7 aa, Artificial Sequence
  phosphopeptide or phosphopeptide variant
KRFSFKF

SEQ ID NO: 692 - protein, 7 aa, Artificial Sequence
  phosphopeptide or phosphopeptide variant
KRFSFKR

SEQ ID NO: 693 - protein, 7 aa, Artificial Sequence
  phosphopeptide or phosphopeptide variant
KRFSFKM

SEQ ID NO: 694 - protein, 7 aa, Artificial Sequence
  phosphopeptide or phosphopeptide variant
RRFSFKK

SEQ ID NO: 695 - protein, 7 aa, Artificial Sequence
  phosphopeptide or phosphopeptide variant
RRFSFKL

SEQ ID NO: 696 - protein, 7 aa, Artificial Sequence
  phosphopeptide or phosphopeptide variant
RRFSFKF

SEQ ID NO: 697 - protein, 7 aa, Artificial Sequence
  phosphopeptide or phosphopeptide variant
RRFSFKR

SEQ ID NO: 698 - protein, 7 aa, Artificial Sequence
  phosphopeptide or phosphopeptide variant
RRFSFKM

SEQ ID NO: 699 - protein, 9 aa, Artificial Sequence
  phosphopeptide or phosphopeptide variant
KRFSFKKSL

SEQ ID NO: 700 - protein, 9 aa, Artificial Sequence
  phosphopeptide or phosphopeptide variant
KRFSFKKSK

SEQ ID NO: 701 - protein, 9 aa, Artificial Sequence
  phosphopeptide or phosphopeptide variant
KRFSFKKSR

SEQ ID NO: 702 - protein, 9 aa, Artificial Sequence
  phosphopeptide or phosphopeptide variant
KRFSFKKSM

SEQ ID NO: 703 - protein, 9 aa, Artificial Sequence
  phosphopeptide or phosphopeptide variant
RRFSFKKSF

SEQ ID NO: 704 - protein, 9 aa, Artificial Sequence
  phosphopeptide or phosphopeptide variant
RRFSFKKSL

SEQ ID NO: 705 - protein, 9 aa, Artificial Sequence
  phosphopeptide or phosphopeptide variant
RRFSFKKSK

SEQ ID NO: 706 - protein, 9 aa, Artificial Sequence
  phosphopeptide or phosphopeptide variant
RRFSFKKSR

SEQ ID NO: 707 - protein, 9 aa, Artificial Sequence
  phosphopeptide or phosphopeptide variant
RRFSFKKSM

SEQ ID NO: 708 - protein, 9 aa, Artificial Sequence
  phosphopeptide or phosphopeptide variant
KRFSGTVRF

SEQ ID NO: 709 - protein, 9 aa, Artificial Sequence
  phosphopeptide or phosphopeptide variant
KRFSGTVRK

SEQ ID NO: 710 - protein, 9 aa, Artificial Sequence
  phosphopeptide or phosphopeptide variant
KRFSGTVRR

SEQ ID NO: 711 - protein, 9 aa, Artificial Sequence
  phosphopeptide or phosphopeptide variant
KRFSGTVRM

SEQ ID NO: 712 - protein, 9 aa, Artificial Sequence
  phosphopeptide or phosphopeptide variant
RRFSGTVRL

SEQ ID NO: 713 - protein, 9 aa, Artificial Sequence
  phosphopeptide or phosphopeptide variant
RRFSGTVRF

SEQ ID NO: 714 - protein, 9 aa, Artificial Sequence
  phosphopeptide or phosphopeptide variant
RRFSGTVRR

SEQ ID NO: 715 - protein, 9 aa, Artificial Sequence
  phosphopeptide or phosphopeptide variant
RRFSGTVRK

SEQ ID NO: 716 - protein, 9 aa, Artificial Sequence
  phosphopeptide or phosphopeptide variant
RRFSGTVRM

SEQ ID NO: 717 - protein, 9 aa, Artificial Sequence
  phosphopeptide or phosphopeptide variant
KRLSPAPQF

SEQ ID NO: 718 - protein, 9 aa, Artificial Sequence
  phosphopeptide or phosphopeptide variant
KRLSPAPQK

SEQ ID NO: 719 - protein, 9 aa, Artificial Sequence
  phosphopeptide or phosphopeptide variant
KRLSPAPQR

SEQ ID NO: 720 - protein, 9 aa, Artificial Sequence
  phosphopeptide or phosphopeptide variant
KRLSPAPQM

SEQ ID NO: 721 - protein, 9 aa, Artificial Sequence
  phosphopeptide or phosphopeptide variant
RRLSPAPQL

SEQ ID NO: 722 - protein, 9 aa, Artificial Sequence
  phosphopeptide or phosphopeptide variant
RRLSPAPQF

SEQ ID NO: 723 - protein, 9 aa, Artificial Sequence
  phosphopeptide or phosphopeptide variant
RRLSPAPQR

SEQ ID NO: 724 - protein, 9 aa, Artificial Sequence
  phosphopeptide or phosphopeptide variant
RRLSPAPQK

SEQ ID NO: 725 - protein, 9 aa, Artificial Sequence
  phosphopeptide or phosphopeptide variant
RRLSPAPQM

SEQ ID NO: 726 - protein, 9 aa, Artificial Sequence
  phosphopeptide or phosphopeptide variant
KRLSVERIL

SEQ ID NO: 727 - protein, 9 aa, Artificial Sequence
  phosphopeptide or phosphopeptide variant
KRLSVERIF

SEQ ID NO: 728 - protein, 9 aa, Artificial Sequence
  phosphopeptide or phosphopeptide variant
KRLSVERIK

SEQ ID NO: 729 - protein, 9 aa, Artificial Sequence
  phosphopeptide or phosphopeptide variant
KRLSVERIR

SEQ ID NO: 730 - protein, 9 aa, Artificial Sequence
  phosphopeptide or phosphopeptide variant
KRLSVERIM

SEQ ID NO: 731 - protein, 9 aa, Artificial Sequence
  phosphopeptide or phosphopeptide variant
RRLSVERIL

SEQ ID NO: 732 - protein, 9 aa, Artificial Sequence
  phosphopeptide or phosphopeptide variant
RRLSVERIF

SEQ ID NO: 733 - protein, 9 aa, Artificial Sequence
  phosphopeptide or phosphopeptide variant
RRLSVERIR

SEQ ID NO: 734 - protein, 9 aa, Artificial Sequence
  phosphopeptide or phosphopeptide variant
RRLSVERIK

SEQ ID NO: 735 - protein, 9 aa, Artificial Sequence
  phosphopeptide or phosphopeptide variant
RRLSVERIM

SEQ ID NO: 736 - protein, 8 aa, Artificial Sequence
  phosphopeptide or phosphopeptide variant
KRMSPKPF

SEQ ID NO: 737 - protein, 8 aa, Artificial Sequence
  phosphopeptide or phosphopeptide variant
KRMSPKPK

SEQ ID NO: 738 - protein, 8 aa, Artificial Sequence
  phosphopeptide or phosphopeptide variant
KRMSPKPR

SEQ ID NO: 739 - protein, 8 aa, Artificial Sequence
  phosphopeptide or phosphopeptide variant
KRMSPKPM

SEQ ID NO: 740 - protein, 8 aa, Artificial Sequence
  phosphopeptide or phosphopeptide variant
RRMSPKPL

SEQ ID NO: 741 - protein, 8 aa, Artificial Sequence
  phosphopeptide or phosphopeptide variant
RRMSPKPF

SEQ ID NO: 742 - protein, 8 aa, Artificial Sequence
  phosphopeptide or phosphopeptide variant
RRMSPKPR

SEQ ID NO: 743 - protein, 8 aa, Artificial Sequence
  phosphopeptide or phosphopeptide variant
RRMSPKPK

SEQ ID NO: 744 - protein, 8 aa, Artificial Sequence
  phosphopeptide or phosphopeptide variant
RRMSPKPM

SEQ ID NO: 745 - protein, 9 aa, Artificial Sequence
  phosphopeptide or phosphopeptide variant
KRYSGNMEL

SEQ ID NO: 746 - protein, 9 aa, Artificial Sequence
  phosphopeptide or phosphopeptide variant
KRYSGNMEF

SEQ ID NO: 747 - protein, 9 aa, Artificial Sequence
  phosphopeptide or phosphopeptide variant
KRYSGNMEK

SEQ ID NO: 748 - protein, 9 aa, Artificial Sequence
  phosphopeptide or phosphopeptide variant
KRYSGNMEM

SEQ ID NO: 749 - protein, 9 aa, Artificial Sequence
  phosphopeptide or phosphopeptide variant
KRYSGNMER

SEQ ID NO: 750 - protein, 9 aa, Artificial Sequence
  phosphopeptide or phosphopeptide variant
RRYSGNMEL

SEQ ID NO: 751 - protein, 9 aa, Artificial Sequence
  phosphopeptide or phosphopeptide variant
RRYSGNMEF

SEQ ID NO: 752 - protein, 9 aa, Artificial Sequence
  phosphopeptide or phosphopeptide variant
RRYSGNMEK

SEQ ID NO: 753 - protein, 9 aa, Artificial Sequence
  phosphopeptide or phosphopeptide variant
RRYSGNMEM

SEQ ID NO: 754 - protein, 9 aa, Artificial Sequence
  phosphopeptide or phosphopeptide variant
RRYSGNMER

SEQ ID NO: 755 - protein, 9 aa, Artificial Sequence
  phosphopeptide or phosphopeptide variant
MRLSRELQF

SEQ ID NO: 756 - protein, 9 aa, Artificial Sequence
  phosphopeptide or phosphopeptide variant
MRLSRELQK

SEQ ID NO: 757 - protein, 9 aa, Artificial Sequence
  phosphopeptide or phosphopeptide variant
MRLSRELQR

SEQ ID NO: 758 - protein, 9 aa, Artificial Sequence
  phosphopeptide or phosphopeptide variant
MRLSRELQM

SEQ ID NO: 759 - protein, 9 aa, Artificial Sequence
  phosphopeptide or phosphopeptide variant
RRLSRELQL

SEQ ID NO: 760 - protein, 9 aa, Artificial Sequence
  phosphopeptide or phosphopeptide variant
RRLSRELQF

SEQ ID NO: 761 - protein, 9 aa, Artificial Sequence
  phosphopeptide or phosphopeptide variant
RRLSRELQK

SEQ ID NO: 762 - protein, 9 aa, Artificial Sequence
  phosphopeptide or phosphopeptide variant
RRLSRELQR

SEQ ID NO: 763 - protein, 9 aa, Artificial Sequence
  phosphopeptide or phosphopeptide variant
RRLSRELQM

SEQ ID NO: 764 - protein, 10 aa, Artificial Sequence
  phosphopeptide or phosphopeptide variant
NRYTNRVVTF

SEQ ID NO: 765 - protein, 10 aa, Artificial Sequence
  phosphopeptide or phosphopeptide variant
NRYTNRVVTK

SEQ ID NO: 766 - protein, 10 aa, Artificial Sequence
  phosphopeptide or phosphopeptide variant
NRYTNRVVTR

SEQ ID NO: 767 - protein, 10 aa, Artificial Sequence
  phosphopeptide or phosphopeptide variant
NRYTNRVVTM

SEQ ID NO: 768 - protein, 10 aa, Artificial Sequence
  phosphopeptide or phosphopeptide variant
RRYTNRVVTL

SEQ ID NO: 769 - protein, 10 aa, Artificial Sequence
  phosphopeptide or phosphopeptide variant
RRYTNRVVTF

SEQ ID NO: 770 - protein, 10 aa, Artificial Sequence
  phosphopeptide or phosphopeptide variant
RRYTNRVVTM

SEQ ID NO: 771 - protein, 10 aa, Artificial Sequence
  phosphopeptide or phosphopeptide variant
RRYTNRVVTR

SEQ ID NO: 772 - protein, 10 aa, Artificial Sequence
  phosphopeptide or phosphopeptide variant
RRYTNRVVTK

SEQ ID NO: 773 - protein, 9 aa, Artificial Sequence
  phosphopeptide or phosphopeptide variant
RRFSPPRRL

SEQ ID NO: 774 - protein, 9 aa, Artificial Sequence
  phosphopeptide or phosphopeptide variant
RRFSPPRRF

SEQ ID NO: 775 - protein, 9 aa, Artificial Sequence
  phosphopeptide or phosphopeptide variant
RRFSPPRRK

SEQ ID NO: 776 - protein, 9 aa, Artificial Sequence
  phosphopeptide or phosphopeptide variant
RRFSPPRRR

SEQ ID NO: 777 - protein, 9 aa, Artificial Sequence
  phosphopeptide or phosphopeptide variant
RRFSPPRRL

SEQ ID NO: 778 - protein, 9 aa, Artificial Sequence
  phosphopeptide or phosphopeptide variant
RRFSPPRRF

SEQ ID NO: 779 - protein, 9 aa, Artificial Sequence
  phosphopeptide or phosphopeptide variant
RRFSPPRRR

SEQ ID NO: 780 - protein, 9 aa, Artificial Sequence
  phosphopeptide or phosphopeptide variant
RRFSPPRRM

SEQ ID NO: 781 - protein, 9 aa, Artificial Sequence
  phosphopeptide or phosphopeptide variant
RRFSPPRRK

SEQ ID NO: 782 - protein, 9 aa, Artificial Sequence
  phosphopeptide or phosphopeptide variant
RRFSRSPIL

SEQ ID NO: 783 - protein, 9 aa, Artificial Sequence
  phosphopeptide or phosphopeptide variant
RRFSRSPIF

SEQ ID NO: 784 - protein, 9 aa, Artificial Sequence
  phosphopeptide or phosphopeptide variant
RRFSRSPIK

SEQ ID NO: 785 - protein, 9 aa, Artificial Sequence
  phosphopeptide or phosphopeptide variant
RRFSRSPIM

SEQ ID NO: 786 - protein, 9 aa, Artificial Sequence
  phosphopeptide or phosphopeptide variant
RRFSRSPIL

SEQ ID NO: 787 - protein, 9 aa, Artificial Sequence
  phosphopeptide or phosphopeptide variant
RRFSRSPIF

SEQ ID NO: 788 - protein, 9 aa, Artificial Sequence
  phosphopeptide or phosphopeptide variant
RRFSRSPIK

SEQ ID NO: 789 - protein, 9 aa, Artificial Sequence
  phosphopeptide or phosphopeptide variant
RRFSRSPIM

SEQ ID NO: 790 - protein, 9 aa, Artificial Sequence
  phosphopeptide or phosphopeptide variant
RRISGVDRL

SEQ ID NO: 791 - protein, 9 aa, Artificial Sequence
  phosphopeptide or phosphopeptide variant
RRISGVDRF

SEQ ID NO: 792 - protein, 9 aa, Artificial Sequence
  phosphopeptide or phosphopeptide variant
RRISGVDRR

SEQ ID NO: 793 - protein, 9 aa, Artificial Sequence
  phosphopeptide or phosphopeptide variant
RRISGVDRM

SEQ ID NO: 794 - protein, 9 aa, Artificial Sequence
  phosphopeptide or phosphopeptide variant
RRISGVDRK

SEQ ID NO: 795 - protein, 9 aa, Artificial Sequence
  phosphopeptide or phosphopeptide variant
RRKSQLDSF

SEQ ID NO: 796 - protein, 9 aa, Artificial Sequence
  phosphopeptide or phosphopeptide variant
RRKSQLDSR

SEQ ID NO: 797 - protein, 9 aa, Artificial Sequence
  phosphopeptide or phosphopeptide variant
RRKSQLDSK

SEQ ID NO: 798 - protein, 9 aa, Artificial Sequence
  phosphopeptide or phosphopeptide variant
RRKSQLDSM

SEQ ID NO: 799 - protein, 9 aa, Artificial Sequence
  phosphopeptide or phosphopeptide variant
RRKSQVAEF

SEQ ID NO: 800 - protein, 9 aa, Artificial Sequence
  phosphopeptide or phosphopeptide variant
RRKSQVAER

SEQ ID NO: 801 - protein, 9 aa, Artificial Sequence
  phosphopeptide or phosphopeptide variant
RRKSQVAEK

SEQ ID NO: 802 - protein, 9 aa, Artificial Sequence
  phosphopeptide or phosphopeptide variant
RRKSQVAEM

SEQ ID NO: 803 - protein, 9 aa, Artificial Sequence
  phosphopeptide or phosphopeptide variant
RRLSADIRF

SEQ ID NO: 804 - protein, 9 aa, Artificial Sequence
  phosphopeptide or phosphopeptide variant
RRLSADIRR

SEQ ID NO: 805 - protein, 9 aa, Artificial Sequence
  phosphopeptide or phosphopeptide variant
RRLSADIRK

SEQ ID NO: 806 - protein, 9 aa, Artificial Sequence
  phosphopeptide or phosphopeptide variant
RRLSADIRM

SEQ ID NO: 807 - protein, 9 aa, Artificial Sequence
  phosphopeptide or phosphopeptide variant
RRLSFLVSL

SEQ ID NO: 808 - protein, 9 aa, Artificial Sequence
  phosphopeptide or phosphopeptide variant
RRLSFLVSF

SEQ ID NO: 809 - protein, 9 aa, Artificial Sequence
  phosphopeptide or phosphopeptide variant
RRLSFLVSR

SEQ ID NO: 810 - protein, 9 aa, Artificial Sequence
  phosphopeptide or phosphopeptide variant
RRLSFLVSM

SEQ ID NO: 811 - protein, 9 aa, Artificial Sequence
  phosphopeptide or phosphopeptide variant
RRLSFLVSK

SEQ ID NO: 812 - protein, 10 aa, Artificial Sequence
  phosphopeptide or phosphopeptide variant
RRLSGGSHSL

SEQ ID NO: 813 - protein, 10 aa, Artificial Sequence
  phosphopeptide or phosphopeptide variant
RRLSGGSHSF

SEQ ID NO: 814 - protein, 10 aa, Artificial Sequence
  phosphopeptide or phosphopeptide variant
RRLSGGSHSR

SEQ ID NO: 815 - protein, 10 aa, Artificial Sequence
  phosphopeptide or phosphopeptide variant
RRLSGGSHSK

SEQ ID NO: 816 - protein, 10 aa, Artificial Sequence
  phosphopeptide or phosphopeptide variant
RRLSGGSHSM

SEQ ID NO: 817 - protein, 10 aa, Artificial Sequence
  phosphopeptide or phosphopeptide variant
RRLSGPLHTF

SEQ ID NO: 818 - protein, 10 aa, Artificial Sequence
  phosphopeptide or phosphopeptide variant
RRLSGPLHTK

SEQ ID NO: 819 - protein, 10 aa, Artificial Sequence
  phosphopeptide or phosphopeptide variant
RRLSGPLHTR

SEQ ID NO: 820 - protein, 10 aa, Artificial Sequence
  phosphopeptide or phosphopeptide variant
RRLSGPLHTM

SEQ ID NO: 821 - protein, 9 aa, Artificial Sequence
  phosphopeptide or phosphopeptide variant
RRMSLLSVL

SEQ ID NO: 822 - protein, 9 aa, Artificial Sequence
  phosphopeptide or phosphopeptide variant
RRMSLLSVF

SEQ ID NO: 823 - protein, 9 aa, Artificial Sequence
  phosphopeptide or phosphopeptide variant
RRMSLLSVK

SEQ ID NO: 824 - protein, 9 aa, Artificial Sequence
  phosphopeptide or phosphopeptide variant
RRMSLLSVR

SEQ ID NO: 825 - protein, 9 aa, Artificial Sequence
  phosphopeptide or phosphopeptide variant
RRMSLLSVM

SEQ ID NO: 826 - protein, 9 aa, Artificial Sequence
  phosphopeptide or phosphopeptide variant
RRNSSERTF

SEQ ID NO: 827 - protein, 9 aa, Artificial Sequence
  phosphopeptide or phosphopeptide variant
RRNSSERTK

SEQ ID NO: 828 - protein, 9 aa, Artificial Sequence
  phosphopeptide or phosphopeptide variant
RRNSSERTR

SEQ ID NO: 829 - protein, 9 aa, Artificial Sequence
  phosphopeptide or phosphopeptide variant
RRNSSERTM

SEQ ID NO: 830 - protein, 9 aa, Artificial Sequence
  phosphopeptide or phosphopeptide variant
RRNSSIVGL

SEQ ID NO: 831 - protein, 9 aa, Artificial Sequence
  phosphopeptide or phosphopeptide variant
RRNSSIVGF

SEQ ID NO: 832 - protein, 9 aa, Artificial Sequence
  phosphopeptide or phosphopeptide variant
RRNSSIVGK

SEQ ID NO: 833 - protein, 9 aa, Artificial Sequence
  phosphopeptide or phosphopeptide variant
RRNSSIVGM

SEQ ID NO: 834 - protein, 10 aa, Artificial Sequence
  phosphopeptide or phosphopeptide variant
RRNSVFQQGL

SEQ ID NO: 835 - protein, 10 aa, Artificial Sequence
  phosphopeptide or phosphopeptide variant
RRNSVFQQGF

SEQ ID NO: 836 - protein, 10 aa, Artificial Sequence
  phosphopeptide or phosphopeptide variant
RRNSVFQQGK

SEQ ID NO: 837 - protein, 10 aa, Artificial Sequence
  phosphopeptide or phosphopeptide variant
RRNSVFQQGR

SEQ ID NO: 838 - protein, 9 aa, Artificial Sequence
  phosphopeptide or phosphopeptide variant
RRPSLVHGL

SEQ ID NO: 839 - protein, 9 aa, Artificial Sequence
  phosphopeptide or phosphopeptide variant
RRPSLVHGF

SEQ ID NO: 840 - protein, 9 aa, Artificial Sequence
  phosphopeptide or phosphopeptide variant
RRPSLVHGK

SEQ ID NO: 841 - protein, 9 aa, Artificial Sequence
  phosphopeptide or phosphopeptide variant
RRPSLVHGR

SEQ ID NO: 842 - protein, 9 aa, Artificial Sequence
  phosphopeptide or phosphopeptide variant
RRPSLVHGM

SEQ ID NO: 843 - protein, 9 aa, Artificial Sequence
  phosphopeptide or phosphopeptide variant
RRPSVFERF

SEQ ID NO: 844 - protein, 9 aa, Artificial Sequence
  phosphopeptide or phosphopeptide variant
RRPSVFERK

SEQ ID NO: 845 - protein, 9 aa, Artificial Sequence
  phosphopeptide or phosphopeptide variant
RRPSVFERM

SEQ ID NO: 846 - protein, 9 aa, Artificial Sequence
  phosphopeptide or phosphopeptide variant
RRPSVFERR

SEQ ID NO: 847 - protein, 9 aa, Artificial Sequence
  phosphopeptide or phosphopeptide variant
RRPSYRKIF

SEQ ID NO: 848 - protein, 9 aa, Artificial Sequence
  phosphopeptide or phosphopeptide variant
RRPSYRKIK

SEQ ID NO: 849 - protein, 9 aa, Artificial Sequence
  phosphopeptide or phosphopeptide variant
RRPSYRKIR

SEQ ID NO: 850 - protein, 9 aa, Artificial Sequence
  phosphopeptide or phosphopeptide variant
RRPSYRKIM

SEQ ID NO: 851 - protein, 9 aa, Artificial Sequence
  phosphopeptide or phosphopeptide variant
RRPSYTLGL

SEQ ID NO: 852 - protein, 9 aa, Artificial Sequence
  phosphopeptide or phosphopeptide variant
RRPSYTLGF

SEQ ID NO: 853 - protein, 9 aa, Artificial Sequence
  phosphopeptide or phosphopeptide variant
RRPSYTLGK

SEQ ID NO: 854 - protein, 9 aa, Artificial Sequence
  phosphopeptide or phosphopeptide variant
RRPSYTLGR

SEQ ID NO: 855 - protein, 9 aa, Artificial Sequence
  phosphopeptide or phosphopeptide variant
RRSSFLQVL

SEQ ID NO: 856 - protein, 9 aa, Artificial Sequence
  phosphopeptide or phosphopeptide variant
RRSSFLQVK

SEQ ID NO: 857 - protein, 9 aa, Artificial Sequence
  phosphopeptide or phosphopeptide variant
RRSSFLQVR

SEQ ID NO: 858 - protein, 9 aa, Artificial Sequence
  phosphopeptide or phosphopeptide variant
RRSSFLQVM

SEQ ID NO: 859 - protein, 9 aa, Artificial Sequence
  phosphopeptide or phosphopeptide variant
RRSSIGLRL

SEQ ID NO: 860 - protein, 9 aa, Artificial Sequence
  phosphopeptide or phosphopeptide variant
RRSSIGLRF

SEQ ID NO: 861 - protein, 9 aa, Artificial Sequence
  phosphopeptide or phosphopeptide variant
RRSSIGLRK

SEQ ID NO: 862 - protein, 9 aa, Artificial Sequence
  phosphopeptide or phosphopeptide variant
RRSSIGLRR

SEQ ID NO: 863 - protein, 9 aa, Artificial Sequence
  phosphopeptide or phosphopeptide variant
RRSSIGLRM

SEQ ID NO: 864 - protein, 9 aa, Artificial Sequence
  phosphopeptide or phosphopeptide variant
RRSSIQSTL

SEQ ID NO: 865 - protein, 9 aa, Artificial Sequence
  phosphopeptide or phosphopeptide variant
RRSSIQSTK

SEQ ID NO: 866 - protein, 9 aa, Artificial Sequence
  phosphopeptide or phosphopeptide variant
RRSSIQSTR

SEQ ID NO: 867 - protein, 9 aa, Artificial Sequence
  phosphopeptide or phosphopeptide variant
RRSSIQSTM

SEQ ID NO: 868 - protein, 9 aa, Artificial Sequence
  phosphopeptide or phosphopeptide variant
RRSSQSWSF

SEQ ID NO: 869 - protein, 9 aa, Artificial Sequence
  phosphopeptide or phosphopeptide variant
RRSSQSWSK

SEQ ID NO: 870 - protein, 9 aa, Artificial Sequence
  phosphopeptide or phosphopeptide variant
RRSSQSWSR

SEQ ID NO: 871 - protein, 9 aa, Artificial Sequence
  phosphopeptide or phosphopeptide variant
RRSSQSWSM

SEQ ID NO: 872 - protein, 9 aa, Artificial Sequence
  phosphopeptide or phosphopeptide variant
RRSSVDLGF

SEQ ID NO: 873 - protein, 9 aa, Artificial Sequence
  phosphopeptide or phosphopeptide variant
RRSSVDLGK

SEQ ID NO: 874 - protein, 9 aa, Artificial Sequence
  phosphopeptide or phosphopeptide variant
RRSSVDLGR

SEQ ID NO: 875 - protein, 9 aa, Artificial Sequence
  phosphopeptide or phosphopeptide variant
RRSSVDLGM

SEQ ID NO: 876 - protein, 9 aa, Artificial Sequence
  phosphopeptide or phosphopeptide variant
RRSSVDLGF

SEQ ID NO: 877 - protein, 9 aa, Artificial Sequence
  phosphopeptide or phosphopeptide variant
RRSSVDLGK

SEQ ID NO: 878 - protein, 9 aa, Artificial Sequence
  phosphopeptide or phosphopeptide variant
RRSSVDLGR

SEQ ID NO: 879 - protein, 9 aa, Artificial Sequence
  phosphopeptide or phosphopeptide variant
RRSSVDLGM

SEQ ID NO: 880 - protein, 9 aa, Artificial Sequence
  phosphopeptide or phosphopeptide variant
RRSSVKVEL

SEQ ID NO: 881 - protein, 9 aa, Artificial Sequence
  phosphopeptide or phosphopeptide variant
RRSSVKVEF

SEQ ID NO: 882 - protein, 9 aa, Artificial Sequence
  phosphopeptide or phosphopeptide variant
RRSSVKVEK

SEQ ID NO: 883 - protein, 9 aa, Artificial Sequence
  phosphopeptide or phosphopeptide variant
RRSSVKVEM

SEQ ID NO: 884 - protein, 9 aa, Artificial Sequence
  phosphopeptide or phosphopeptide variant
RRSSVKVER

SEQ ID NO: 885 - protein, 9 aa, Artificial Sequence
  phosphopeptide or phosphopeptide variant
RRVVQRSSF

SEQ ID NO: 886 - protein, 9 aa, Artificial Sequence
  phosphopeptide or phosphopeptide variant
RRVVQRSSK

SEQ ID NO: 887 - protein, 9 aa, Artificial Sequence
  phosphopeptide or phosphopeptide variant
RRVVQRSSR

SEQ ID NO: 888 - protein, 9 aa, Artificial Sequence
  phosphopeptide or phosphopeptide variant
RRVVQRSSM

SEQ ID NO: 889 - protein, 9 aa, Artificial Sequence
  phosphopeptide or phosphopeptide variant
RRYSPPIQF

SEQ ID NO: 890 - protein, 9 aa, Artificial Sequence
  phosphopeptide or phosphopeptide variant
RRYSPPIQL

SEQ ID NO: 891 - protein, 9 aa, Artificial Sequence
  phosphopeptide or phosphopeptide variant
RRYSPPIQK

SEQ ID NO: 892 - protein, 9 aa, Artificial Sequence
  phosphopeptide or phosphopeptide variant
RRYSPPIQM

SEQ ID NO: 893 - protein, 8 aa, Artificial Sequence
  phosphopeptide or phosphopeptide variant
SRLTHLSF

SEQ ID NO: 894 - protein, 8 aa, Artificial Sequence
  phosphopeptide or phosphopeptide variant
SRLTHLSK

SEQ ID NO: 895 - protein, 8 aa, Artificial Sequence
  phosphopeptide or phosphopeptide variant
SRLTHLSR

SEQ ID NO: 896 - protein, 8 aa, Artificial Sequence
  phosphopeptide or phosphopeptide variant
SRLTHLSM

SEQ ID NO: 897 - protein, 8 aa, Artificial Sequence
  phosphopeptide or phosphopeptide variant
RRLTHLSL

SEQ ID NO: 898 - protein, 8 aa, Artificial Sequence
  phosphopeptide or phosphopeptide variant
RRLTHLSF

SEQ ID NO: 899 - protein, 8 aa, Artificial Sequence
  phosphopeptide or phosphopeptide variant
RRLTHLSK

SEQ ID NO: 900 - protein, 8 aa, Artificial Sequence
  phosphopeptide or phosphopeptide variant
RRLTHLSR

SEQ ID NO: 901 - protein, 8 aa, Artificial Sequence
  phosphopeptide or phosphopeptide variant
RRLTHLSM

SEQ ID NO: 902 - protein, 9 aa, Artificial Sequence
  phosphopeptide or phosphopeptide variant
SRMSPKAQL

SEQ ID NO: 903 - protein, 9 aa, Artificial Sequence
  phosphopeptide or phosphopeptide variant
SRMSPKAQF

SEQ ID NO: 904 - protein, 9 aa, Artificial Sequence
  phosphopeptide or phosphopeptide variant
SRMSPKAQK

SEQ ID NO: 905 - protein, 9 aa, Artificial Sequence
  phosphopeptide or phosphopeptide variant
SRMSPKAQM

SEQ ID NO: 906 - protein, 9 aa, Artificial Sequence
  phosphopeptide or phosphopeptide variant
RRMSPKAQL

SEQ ID NO: 907 - protein, 9 aa, Artificial Sequence
  phosphopeptide or phosphopeptide variant
RRMSPKAQF

SEQ ID NO: 908 - protein, 9 aa, Artificial Sequence
  phosphopeptide or phosphopeptide variant
RRMSPKAQR

SEQ ID NO: 909 - protein, 9 aa, Artificial Sequence
  phosphopeptide or phosphopeptide variant
RRMSPKAQM

SEQ ID NO: 910 - protein, 9 aa, Artificial Sequence
  phosphopeptide or phosphopeptide variant
RRMSPKAQK

SEQ ID NO: 911 - protein, 9 aa, Artificial Sequence
  phosphopeptide or phosphopeptide variant
SRTSPITRL

SEQ ID NO: 912 - protein, 9 aa, Artificial Sequence
  phosphopeptide or phosphopeptide variant
SRTSPITRF

SEQ ID NO: 913 - protein, 9 aa, Artificial Sequence
  phosphopeptide or phosphopeptide variant
SRTSPITRK

SEQ ID NO: 914 - protein, 9 aa, Artificial Sequence
  phosphopeptide or phosphopeptide variant
SRTSPITRM

SEQ ID NO: 915 - protein, 9 aa, Artificial Sequence
  phosphopeptide or phosphopeptide variant
RRTSPITRL

SEQ ID NO: 916 - protein, 9 aa, Artificial Sequence
  phosphopeptide or phosphopeptide variant
RRTSPITRR

SEQ ID NO: 917 - protein, 9 aa, Artificial Sequence
  phosphopeptide or phosphopeptide variant
RRTSPITRF

SEQ ID NO: 918 - protein, 9 aa, Artificial Sequence
  phosphopeptide or phosphopeptide variant
RRTSPITRM

SEQ ID NO: 919 - protein, 9 aa, Artificial Sequence
  phosphopeptide or phosphopeptide variant
RRTSPITRK

SEQ ID NO: 920 - protein, 10 aa, Artificial Sequence
  phosphopeptide or phosphopeptide variant
SRYSRSPYSL

SEQ ID NO: 921 - protein, 10 aa, Artificial Sequence
  phosphopeptide or phosphopeptide variant
SRYSRSPYSF

SEQ ID NO: 922 - protein, 10 aa, Artificial Sequence
  phosphopeptide or phosphopeptide variant
SRYSRSPYSK

SEQ ID NO: 923 - protein, 10 aa, Artificial Sequence
  phosphopeptide or phosphopeptide variant
SRYSRSPYSM

SEQ ID NO: 924 - protein, 10 aa, Artificial Sequence
  phosphopeptide or phosphopeptide variant
RRYSRSPYSL

SEQ ID NO: 925 - protein, 10 aa, Artificial Sequence
  phosphopeptide or phosphopeptide variant
RRYSRSPYSF

SEQ ID NO: 926 - protein, 10 aa, Artificial Sequence
  phosphopeptide or phosphopeptide variant
RRYSRSPYSR

SEQ ID NO: 927 - protein, 10 aa, Artificial Sequence
  phosphopeptide or phosphopeptide variant
RRYSRSPYSM

SEQ ID NO: 928 - protein, 10 aa, Artificial Sequence
  phosphopeptide or phosphopeptide variant
RRYSRSPYSK

SEQ ID NO: 929 - protein, 10 aa, Artificial Sequence
  phosphopeptide or phosphopeptide variant
SRYSRSPYSL

SEQ ID NO: 930 - protein, 10 aa, Artificial Sequence
  phosphopeptide or phosphopeptide variant
SRYSRSPYSF

SEQ ID NO: 931 - protein, 10 aa, Artificial Sequence
  phosphopeptide or phosphopeptide variant
SRYSRSPYSK

SEQ ID NO: 932 - protein, 10 aa, Artificial Sequence
  phosphopeptide or phosphopeptide variant
SRYSRSPYSM

SEQ ID NO: 933 - protein, 10 aa, Artificial Sequence
  phosphopeptide or phosphopeptide variant
RRYSRSPYSR

SEQ ID NO: 934 - protein, 10 aa, Artificial Sequence
  phosphopeptide or phosphopeptide variant
RRYSRSPYSL

SEQ ID NO: 935 - protein, 10 aa, Artificial Sequence
  phosphopeptide or phosphopeptide variant
RRYSRSPYSF

SEQ ID NO: 936 - protein, 10 aa, Artificial Sequence
  phosphopeptide or phosphopeptide variant
RRYSRSPYSK

SEQ ID NO: 937 - protein, 10 aa, Artificial Sequence
  phosphopeptide or phosphopeptide variant
RRYSRSPYSM

SEQ ID NO: 938 - protein, 10 aa, Artificial Sequence
  phosphopeptide or phosphopeptide variant
SRYSRSPYSL

SEQ ID NO: 939 - protein, 10 aa, Artificial Sequence
  phosphopeptide or phosphopeptide variant
SRYSRSPYSF

SEQ ID NO: 940 - protein, 10 aa, Artificial Sequence
  phosphopeptide or phosphopeptide variant
SRYSRSPYSK

SEQ ID NO: 941 - protein, 10 aa, Artificial Sequence
  phosphopeptide or phosphopeptide variant
SRYSRSPYSM

SEQ ID NO: 942 - protein, 10 aa, Artificial Sequence
  phosphopeptide or phosphopeptide variant
RRYSRSPYSR

SEQ ID NO: 943 - protein, 10 aa, Artificial Sequence
  phosphopeptide or phosphopeptide variant
RRYSRSPYSL

SEQ ID NO: 944 - protein, 10 aa, Artificial Sequence
  phosphopeptide or phosphopeptide variant
RRYSRSPYSF

SEQ ID NO: 945 - protein, 10 aa, Artificial Sequence
  phosphopeptide or phosphopeptide variant
RRYSRSPYSM

SEQ ID NO: 946 - protein, 10 aa, Artificial Sequence
  phosphopeptide or phosphopeptide variant
RRYSRSPYSK

SEQ ID NO: 947 - protein, 9 aa, Artificial Sequence
  phosphopeptide or phosphopeptide variant
EPKRRSARV

SEQ ID NO: 948 - protein, 9 aa, Artificial Sequence
  phosphopeptide or phosphopeptide variant
EPKRRSARM

SEQ ID NO: 949 - protein, 9 aa, Artificial Sequence
  phosphopeptide or phosphopeptide variant
EPKRRSARF

SEQ ID NO: 950 - protein, 9 aa, Artificial Sequence
  phosphopeptide or phosphopeptide variant
EPRSPSHSL

SEQ ID NO: 951 - protein, 9 aa, Artificial Sequence
  phosphopeptide or phosphopeptide variant
EPRSPSHSV

SEQ ID NO: 952 - protein, 9 aa, Artificial Sequence
  phosphopeptide or phosphopeptide variant
EPRSPSHSF

SEQ ID NO: 953 - protein, 10 aa, Artificial Sequence
  phosphopeptide or phosphopeptide variant
FRRSPTKSSV

SEQ ID NO: 954 - protein, 10 aa, Artificial Sequence
  phosphopeptide or phosphopeptide variant
FRRSPTKSSM

SEQ ID NO: 955 - protein, 10 aa, Artificial Sequence
  phosphopeptide or phosphopeptide variant
FRRSPTKSSF

SEQ ID NO: 956 - protein, 10 aa, Artificial Sequence
  phosphopeptide or phosphopeptide variant
FPRSPTKSSL

SEQ ID NO: 957 - protein, 10 aa, Artificial Sequence
  phosphopeptide or phosphopeptide variant
FPRSPTKSSV

SEQ ID NO: 958 - protein, 10 aa, Artificial Sequence
  phosphopeptide or phosphopeptide variant
FPRSPTKSSM

SEQ ID NO: 959 - protein, 10 aa, Artificial Sequence
  phosphopeptide or phosphopeptide variant
FPRSPTKSSF

SEQ ID NO: 960 - protein, 11 aa, Artificial Sequence
  phosphopeptide or phosphopeptide variant
FRSPTKSSLDL

SEQ ID NO: 961 - protein, 11 aa, Artificial Sequence
  phosphopeptide or phosphopeptide variant
FRSPTKSSLDV

SEQ ID NO: 962 - protein, 11 aa, Artificial Sequence
  phosphopeptide or phosphopeptide variant
FRSPTKSSLDF

SEQ ID NO: 963 - protein, 11 aa, Artificial Sequence
  phosphopeptide or phosphopeptide variant
FRSPTKSSLDM

SEQ ID NO: 964 - protein, 12 aa, Artificial Sequence
  phosphopeptide or phosphopeptide variant
FPRSPTKSSLDL

SEQ ID NO: 965 - protein, 12 aa, Artificial Sequence
  phosphopeptide or phosphopeptide variant
FPRSPTKSSLDV

SEQ ID NO: 966 - protein, 12 aa, Artificial Sequence
  phosphopeptide or phosphopeptide variant
FPRSPTKSSLDM

SEQ ID NO: 967 - protein, 12 aa, Artificial Sequence
  phosphopeptide or phosphopeptide variant
FPRSPTKSSLDF

SEQ ID NO: 968 - protein, 8 aa, Artificial Sequence
  phosphopeptide or phosphopeptide variant
FSISPVRV

SEQ ID NO: 969 - protein, 8 aa, Artificial Sequence
  phosphopeptide or phosphopeptide variant
FSISPVRM

SEQ ID NO: 970 - protein, 8 aa, Artificial Sequence
  phosphopeptide or phosphopeptide variant
FSISPVRF

SEQ ID NO: 971 - protein, 8 aa, Artificial Sequence
  phosphopeptide or phosphopeptide variant
FPISPVRL

SEQ ID NO: 972 - protein, 8 aa, Artificial Sequence
  phosphopeptide or phosphopeptide variant
FPISPVRV

SEQ ID NO: 973 - protein, 8 aa, Artificial Sequence
  phosphopeptide or phosphopeptide variant
FPISPVRM

SEQ ID NO: 974 - protein, 8 aa, Artificial Sequence
  phosphopeptide or phosphopeptide variant
FPISPVRF

SEQ ID NO: 975 - protein, 10 aa, Artificial Sequence
  phosphopeptide or phosphopeptide variant
GPRSASLLSV

SEQ ID NO: 976 - protein, 10 aa, Artificial Sequence
  phosphopeptide or phosphopeptide variant
GPRSASLLSF

SEQ ID NO: 977 - protein, 10 aa, Artificial Sequence
  phosphopeptide or phosphopeptide variant
GPRSASLLSM

SEQ ID NO: 978 - protein, 10 aa, Artificial Sequence
  phosphopeptide or phosphopeptide variant
GPRSASLLSV

SEQ ID NO: 979 - protein, 10 aa, Artificial Sequence
  phosphopeptide or phosphopeptide variant
GPRSASLLSF

SEQ ID NO: 980 - protein, 10 aa, Artificial Sequence
  phosphopeptide or phosphopeptide variant
GPRSASLLSM

SEQ ID NO: 981 - protein, 9 aa, Artificial Sequence
  phosphopeptide or phosphopeptide variant
HPRSPTPTV

SEQ ID NO: 982 - protein, 9 aa, Artificial Sequence
  phosphopeptide or phosphopeptide variant
HPRSPTPTF

SEQ ID NO: 983 - protein, 9 aa, Artificial Sequence
  phosphopeptide or phosphopeptide variant
HPRSPTPTM

SEQ ID NO: 984 - protein, 11 aa, Artificial Sequence
  phosphopeptide or phosphopeptide variant
KPASPKFIVTV

SEQ ID NO: 985 - protein, 11 aa, Artificial Sequence
  phosphopeptide or phosphopeptide variant
KPASPKFIVTF

SEQ ID NO: 986 - protein, 11 aa, Artificial Sequence
  phosphopeptide or phosphopeptide variant
KPASPKFIVTM

SEQ ID NO: 987 - protein, 9 aa, Artificial Sequence
  phosphopeptide or phosphopeptide variant
KPPHSPLVM

SEQ ID NO: 988 - protein, 9 aa, Artificial Sequence
  phosphopeptide or phosphopeptide variant
KPPHSPLVV

SEQ ID NO: 989 - protein, 9 aa, Artificial Sequence
  phosphopeptide or phosphopeptide variant
KPPHSPLVF

SEQ ID NO: 990 - protein, 10 aa, Artificial Sequence
  phosphopeptide or phosphopeptide variant
KPPSPEHQSV

SEQ ID NO: 991 - protein, 10 aa, Artificial Sequence
  phosphopeptide or phosphopeptide variant
KPPSPEHQSF

SEQ ID NO: 992 - protein, 10 aa, Artificial Sequence
  phosphopeptide or phosphopeptide variant
KPPSPEHQSM

SEQ ID NO: 993 - protein, 10 aa, Artificial Sequence
  phosphopeptide or phosphopeptide variant
KPPSPSPIEL

SEQ ID NO: 994 - protein, 10 aa, Artificial Sequence
  phosphopeptide or phosphopeptide variant
KPPSPSPIEV

SEQ ID NO: 995 - protein, 10 aa, Artificial Sequence
  phosphopeptide or phosphopeptide variant
KPPSPSPIEF

SEQ ID NO: 996 - protein, 9 aa, Artificial Sequence
  phosphopeptide or phosphopeptide variant
KPPTPGASL

SEQ ID NO: 997 - protein, 9 aa, Artificial Sequence
  phosphopeptide or phosphopeptide variant
KPPTPGASV

SEQ ID NO: 998 - protein, 9 aa, Artificial Sequence
  phosphopeptide or phosphopeptide variant
KPPTPGASM

SEQ ID NO: 999 - protein, 9 aa, Artificial Sequence
  phosphopeptide or phosphopeptide variant
KPPYRSHSV

SEQ ID NO: 1000 - protein, 9 aa, Artificial Sequence
  phosphopeptide or phosphopeptide variant
KPPYRSHSF

SEQ ID NO: 1001 - protein, 9 aa, Artificial Sequence
  phosphopeptide or phosphopeptide variant
KPPYRSHSM

SEQ ID NO: 1002 - protein, 9 aa, Artificial Sequence
  phosphopeptide or phosphopeptide variant
KPQTRGKTL

SEQ ID NO: 1003 - protein, 9 aa, Artificial Sequence
  phosphopeptide or phosphopeptide variant
KPQTRGKTV

SEQ ID NO: 1004 - protein, 9 aa, Artificial Sequence
  phosphopeptide or phosphopeptide variant
KPQTRGKTM

SEQ ID NO: 1005 - protein, 9 aa, Artificial Sequence
  phosphopeptide or phosphopeptide variant
KPRPPPLSL

SEQ ID NO: 1006 - protein, 9 aa, Artificial Sequence
  phosphopeptide or phosphopeptide variant
KPRPPPLSV

SEQ ID NO: 1007 - protein, 9 aa, Artificial Sequence
  phosphopeptide or phosphopeptide variant
KPRPPPLSF

SEQ ID NO: 1008 - protein, 9 aa, Artificial Sequence
  phosphopeptide or phosphopeptide variant
KPRPPPLSM

SEQ ID NO: 1009 - protein, 9 aa, Artificial Sequence
  phosphopeptide or phosphopeptide variant
KPRSPDHVV

SEQ ID NO: 1010 - protein, 9 aa, Artificial Sequence
  phosphopeptide or phosphopeptide variant
KPRSPDHVF

SEQ ID NO: 1011 - protein, 9 aa, Artificial Sequence
  phosphopeptide or phosphopeptide variant
KPRSPDHVM

SEQ ID NO: 1012 - protein, 10 aa, Artificial Sequence
  phosphopeptide or phosphopeptide variant
KPRSPPRALM

SEQ ID NO: 1013 - protein, 10 aa, Artificial Sequence
  phosphopeptide or phosphopeptide variant
KPRSPPRALL

SEQ ID NO: 1014 - protein, 10 aa, Artificial Sequence
  phosphopeptide or phosphopeptide variant
KPRSPPRALF

SEQ ID NO: 1015 - protein, 11 aa, Artificial Sequence
  phosphopeptide or phosphopeptide variant
KPRSPPRALVV

SEQ ID NO: 1016 - protein, 11 aa, Artificial Sequence
  phosphopeptide or phosphopeptide variant
KPRSPPRALVF

SEQ ID NO: 1017 - protein, 11 aa, Artificial Sequence
  phosphopeptide or phosphopeptide variant
KPRSPPRALVM

SEQ ID NO: 1018 - protein, 9 aa, Artificial Sequence
  phosphopeptide or phosphopeptide variant
KPYSPLASV

SEQ ID NO: 1019 - protein, 9 aa, Artificial Sequence
  phosphopeptide or phosphopeptide variant
KPYSPLASF

SEQ ID NO: 1020 - protein, 9 aa, Artificial Sequence
  phosphopeptide or phosphopeptide variant
KPYSPLASM

SEQ ID NO: 1021 - protein, 9 aa, Artificial Sequence
  phosphopeptide or phosphopeptide variant
LPIFSRLSL

SEQ ID NO: 1022 - protein, 9 aa, Artificial Sequence
  phosphopeptide or phosphopeptide variant
LPIFSRLSV

SEQ ID NO: 1023 - protein, 9 aa, Artificial Sequence
  phosphopeptide or phosphopeptide variant
LPIFSRLSF

SEQ ID NO: 1024 - protein, 9 aa, Artificial Sequence
  phosphopeptide or phosphopeptide variant
LPIFSRLSM

SEQ ID NO: 1025 - protein, 10 aa, Artificial Sequence
  phosphopeptide or phosphopeptide variant
MPRQPSATRV

SEQ ID NO: 1026 - protein, 10 aa, Artificial Sequence
  phosphopeptide or phosphopeptide variant
MPRQPSATRF

SEQ ID NO: 1027 - protein, 10 aa, Artificial Sequence
  phosphopeptide or phosphopeptide variant
MPRQPSATRM

SEQ ID NO: 1028 - protein, 11 aa, Artificial Sequence
  phosphopeptide or phosphopeptide variant
QPRSPGPDYSV

SEQ ID NO: 1029 - protein, 11 aa, Artificial Sequence
  phosphopeptide or phosphopeptide variant
QPRSPGPDYSF

SEQ ID NO: 1030 - protein, 11 aa, Artificial Sequence
  phosphopeptide or phosphopeptide variant
QPRSPGPDYSM

SEQ ID NO: 1031 - protein, 10 aa, Artificial Sequence
  phosphopeptide or phosphopeptide variant
QPRTPSPLVV

SEQ ID NO: 1032 - protein, 10 aa, Artificial Sequence
  phosphopeptide or phosphopeptide variant
QPRTPSPLVF

SEQ ID NO: 1033 - protein, 10 aa, Artificial Sequence
  phosphopeptide or phosphopeptide variant
QPRTPSPLVM

SEQ ID NO: 1034 - protein, 10 aa, Artificial Sequence
  phosphopeptide or phosphopeptide variant
QPRTPSPLVV

SEQ ID NO: 1035 - protein, 10 aa, Artificial Sequence
  phosphopeptide or phosphopeptide variant
QPRTPSPLVF

SEQ ID NO: 1036 - protein, 10 aa, Artificial Sequence
  phosphopeptide or phosphopeptide variant
QPRTPSPLVM

SEQ ID NO: 1037 - protein, 9 aa, Artificial Sequence
  phosphopeptide or phosphopeptide variant
RAPSPSSRL

SEQ ID NO: 1038 - protein, 9 aa, Artificial Sequence
  phosphopeptide or phosphopeptide variant
RAPSPSSRV

SEQ ID NO: 1039 - protein, 9 aa, Artificial Sequence
  phosphopeptide or phosphopeptide variant
RAPSPSSRF

SEQ ID NO: 1040 - protein, 9 aa, Artificial Sequence
  phosphopeptide or phosphopeptide variant
RPPSPSSRM

SEQ ID NO: 1041 - protein, 9 aa, Artificial Sequence
  phosphopeptide or phosphopeptide variant
RPPSPSSRL

SEQ ID NO: 1042 - protein, 9 aa, Artificial Sequence
  phosphopeptide or phosphopeptide variant
RPPSPSSRV

SEQ ID NO: 1043 - protein, 9 aa, Artificial Sequence
  phosphopeptide or phosphopeptide variant
RPPSPSSRF

SEQ ID NO: 1044 - protein, 11 aa, Artificial Sequence
  phosphopeptide or phosphopeptide variant
RPASARAQPGV

SEQ ID NO: 1045 - protein, 11 aa, Artificial Sequence
  phosphopeptide or phosphopeptide variant
RPASARAQPGF

SEQ ID NO: 1046 - protein, 11 aa, Artificial Sequence
  phosphopeptide or phosphopeptide variant
RPASARAQPGM

SEQ ID NO: 1047 - protein, 10 aa, Artificial Sequence
  phosphopeptide or phosphopeptide variant
RPATGGPGVL

SEQ ID NO: 1048 - protein, 10 aa, Artificial Sequence
  phosphopeptide or phosphopeptide variant
RPATGGPGVV

SEQ ID NO: 1049 - protein, 10 aa, Artificial Sequence
  phosphopeptide or phosphopeptide variant
RPATGGPGVF

SEQ ID NO: 1050 - protein, 10 aa, Artificial Sequence
  phosphopeptide or phosphopeptide variant
RPATGGPGVM

SEQ ID NO: 1051 - protein, 11 aa, Artificial Sequence
  phosphopeptide or phosphopeptide variant
RPDSRLGKTEV

SEQ ID NO: 1052 - protein, 11 aa, Artificial Sequence
  phosphopeptide or phosphopeptide variant
RPDSRLGKTEF

SEQ ID NO: 1053 - protein, 11 aa, Artificial Sequence
  phosphopeptide or phosphopeptide variant
RPDSRLGKTEM

SEQ ID NO: 1054 - protein, 9 aa, Artificial Sequence
  phosphopeptide or phosphopeptide variant
RPHSPEKAL

SEQ ID NO: 1055 - protein, 9 aa, Artificial Sequence
  phosphopeptide or phosphopeptide variant
RPHSPEKAV

SEQ ID NO: 1056 - protein, 9 aa, Artificial Sequence
  phosphopeptide or phosphopeptide variant
RPHSPEKAM

SEQ ID NO: 1057 - protein, 10 aa, Artificial Sequence
  phosphopeptide or phosphopeptide variant
RPITPPRNSL

SEQ ID NO: 1058 - protein, 10 aa, Artificial Sequence
  phosphopeptide or phosphopeptide variant
RPITPPRNSV

SEQ ID NO: 1059 - protein, 10 aa, Artificial Sequence
  phosphopeptide or phosphopeptide variant
RPITPPRNSF

SEQ ID NO: 1060 - protein, 10 aa, Artificial Sequence
  phosphopeptide or phosphopeptide variant
RPITPPRNSM

SEQ ID NO: 1061 - protein, 9 aa, Artificial Sequence
  phosphopeptide or phosphopeptide variant
RPISPGLSL

SEQ ID NO: 1062 - protein, 9 aa, Artificial Sequence
  phosphopeptide or phosphopeptide variant
RPISPGLSV

SEQ ID NO: 1063 - protein, 9 aa, Artificial Sequence
  phosphopeptide or phosphopeptide variant
RPISPGLSF

SEQ ID NO: 1064 - protein, 9 aa, Artificial Sequence
  phosphopeptide or phosphopeptide variant
RPISPGLSM

SEQ ID NO: 1065 - protein, 9 aa, Artificial Sequence
  phosphopeptide or phosphopeptide variant
RPKLSSPAV

SEQ ID NO: 1066 - protein, 9 aa, Artificial Sequence
  phosphopeptide or phosphopeptide variant
RPKLSSPAF

SEQ ID NO: 1067 - protein, 9 aa, Artificial Sequence
  phosphopeptide or phosphopeptide variant
RPKLSSPAM

SEQ ID NO: 1068 - protein, 9 aa, Artificial Sequence
  phosphopeptide or phosphopeptide variant
RPKPSSSPL

SEQ ID NO: 1069 - protein, 9 aa, Artificial Sequence
  phosphopeptide or phosphopeptide variant
RPKPSSSPF

SEQ ID NO: 1070 - protein, 9 aa, Artificial Sequence
  phosphopeptide or phosphopeptide variant
RPKPSSSPM

SEQ ID NO: 1071 - protein, 10 aa, Artificial Sequence
  phosphopeptide or phosphopeptide variant
RPNSPSPTAV

SEQ ID NO: 1072 - protein, 10 aa, Artificial Sequence
  phosphopeptide or phosphopeptide variant
RPNSPSPTAF

SEQ ID NO: 1073 - protein, 10 aa, Artificial Sequence
  phosphopeptide or phosphopeptide variant
RPNSPSPTAM

SEQ ID NO: 1074 - protein, 10 aa, Artificial Sequence
  phosphopeptide or phosphopeptide variant
RPPPPPDTPL

SEQ ID NO: 1075 - protein, 10 aa, Artificial Sequence
  phosphopeptide or phosphopeptide variant
RPPPPPDTPV

SEQ ID NO: 1076 - protein, 10 aa, Artificial Sequence
  phosphopeptide or phosphopeptide variant
RPPPPPDTPF

SEQ ID NO: 1077 - protein, 10 aa, Artificial Sequence
  phosphopeptide or phosphopeptide variant
RPPPPPDTPM

SEQ ID NO: 1078 - protein, 9 aa, Artificial Sequence
  phosphopeptide or phosphopeptide variant
RPPSPGPVV

SEQ ID NO: 1079 - protein, 9 aa, Artificial Sequence
  phosphopeptide or phosphopeptide variant
RPPSPGPVF

SEQ ID NO: 1080 - protein, 9 aa, Artificial Sequence
  phosphopeptide or phosphopeptide variant
RPPSPGPVM

SEQ ID NO: 1081 - protein, 10 aa, Artificial Sequence
  phosphopeptide or phosphopeptide variant
RPQRATSNVV

SEQ ID NO: 1082 - protein, 10 aa, Artificial Sequence
  phosphopeptide or phosphopeptide variant
RPQRATSNVL

SEQ ID NO: 1083 - protein, 10 aa, Artificial Sequence
  phosphopeptide or phosphopeptide variant
RPQRATSNVM

SEQ ID NO: 1084 - protein, 11 aa, Artificial Sequence
  phosphopeptide or phosphopeptide variant
RPRANSGGVDV

SEQ ID NO: 1085 - protein, 11 aa, Artificial Sequence
  phosphopeptide or phosphopeptide variant
RPRANSGGVDF

SEQ ID NO: 1086 - protein, 11 aa, Artificial Sequence
  phosphopeptide or phosphopeptide variant
RPRANSGGVDM

SEQ ID NO: 1087 - protein, 9 aa, Artificial Sequence
  phosphopeptide or phosphopeptide variant
RPRGSQSLV

SEQ ID NO: 1088 - protein, 9 aa, Artificial Sequence
  phosphopeptide or phosphopeptide variant
RPRGSQSLF

SEQ ID NO: 1089 - protein, 9 aa, Artificial Sequence
  phosphopeptide or phosphopeptide variant
RPRGSQSLM

SEQ ID NO: 1090 - protein, 10 aa, Artificial Sequence
  phosphopeptide or phosphopeptide variant
RPRPHSAPSV

SEQ ID NO: 1091 - protein, 10 aa, Artificial Sequence
  phosphopeptide or phosphopeptide variant
RPRPHSAPSF

SEQ ID NO: 1092 - protein, 10 aa, Artificial Sequence
  phosphopeptide or phosphopeptide variant
RPRPHSAPSM

SEQ ID NO: 1093 - protein, 8 aa, Artificial Sequence
  phosphopeptide or phosphopeptide variant
RPRSAVLV

SEQ ID NO: 1094 - protein, 8 aa, Artificial Sequence
  phosphopeptide or phosphopeptide variant
RPRSAVLF

SEQ ID NO: 1095 - protein, 8 aa, Artificial Sequence
  phosphopeptide or phosphopeptide variant
RPRSAVLM

SEQ ID NO: 1096 - protein, 9 aa, Artificial Sequence
  phosphopeptide or phosphopeptide variant
RPRSLEVTL

SEQ ID NO: 1097 - protein, 9 aa, Artificial Sequence
  phosphopeptide or phosphopeptide variant
RPRSLEVTV

SEQ ID NO: 1098 - protein, 9 aa, Artificial Sequence
  phosphopeptide or phosphopeptide variant
RPRSLEVTF

SEQ ID NO: 1099 - protein, 9 aa, Artificial Sequence
  phosphopeptide or phosphopeptide variant
RPRSLEVTM

SEQ ID NO: 1100 - protein, 9 aa, Artificial Sequence
  phosphopeptide or phosphopeptide variant
RPRSPAARV

SEQ ID NO: 1101 - protein, 9 aa, Artificial Sequence
  phosphopeptide or phosphopeptide variant
RPRSPAARF

SEQ ID NO: 1102 - protein, 9 aa, Artificial Sequence
  phosphopeptide or phosphopeptide variant
RPRSPAARM

SEQ ID NO: 1103 - protein, 10 aa, Artificial Sequence
  phosphopeptide or phosphopeptide variant
RPRSPRENSL

SEQ ID NO: 1104 - protein, 10 aa, Artificial Sequence
  phosphopeptide or phosphopeptide variant
RPRSPRENSV

SEQ ID NO: 1105 - protein, 10 aa, Artificial Sequence
  phosphopeptide or phosphopeptide variant
RPRSPRENSF

SEQ ID NO: 1106 - protein, 10 aa, Artificial Sequence
  phosphopeptide or phosphopeptide variant
RPRSPRENSM

SEQ ID NO: 1107 - protein, 9 aa, Artificial Sequence
  phosphopeptide or phosphopeptide variant
RPRSPSPIL

SEQ ID NO: 1108 - protein, 9 aa, Artificial Sequence
  phosphopeptide or phosphopeptide variant
RPRSPSPIV

SEQ ID NO: 1109 - protein, 9 aa, Artificial Sequence
  phosphopeptide or phosphopeptide variant
RPRSPSPIF

SEQ ID NO: 1110 - protein, 9 aa, Artificial Sequence
  phosphopeptide or phosphopeptide variant
RPRSPSPIM

SEQ ID NO: 1111 - protein, 8 aa, Artificial Sequence
  phosphopeptide or phosphopeptide variant
RPRSPTGL

SEQ ID NO: 1112 - protein, 8 aa, Artificial Sequence
  phosphopeptide or phosphopeptide variant
RPRSPTGV

SEQ ID NO: 1113 - protein, 8 aa, Artificial Sequence
  phosphopeptide or phosphopeptide variant
RPRSPTGF

SEQ ID NO: 1114 - protein, 8 aa, Artificial Sequence
  phosphopeptide or phosphopeptide variant
RPRSPTGM

SEQ ID NO: 1115 - protein, 12 aa, Artificial Sequence
  phosphopeptide or phosphopeptide variant
RPRSPTGPSNSL

SEQ ID NO: 1116 - protein, 12 aa, Artificial Sequence
  phosphopeptide or phosphopeptide variant
RPRSPTGPSNSV

SEQ ID NO: 1117 - protein, 12 aa, Artificial Sequence
  phosphopeptide or phosphopeptide variant
RPRSPTGPSNSM

SEQ ID NO: 1118 - protein, 10 aa, Artificial Sequence
  phosphopeptide or phosphopeptide variant
RPRPVSPSSV

SEQ ID NO: 1119 - protein, 10 aa, Artificial Sequence
  phosphopeptide or phosphopeptide variant
RPRPVSPSSF

SEQ ID NO: 1120 - protein, 10 aa, Artificial Sequence
  phosphopeptide or phosphopeptide variant
RPRPVSPSSM

SEQ ID NO: 1121 - protein, 8 aa, Artificial Sequence
  phosphopeptide or phosphopeptide variant
RPSSLPDV

SEQ ID NO: 1122 - protein, 8 aa, Artificial Sequence
  phosphopeptide or phosphopeptide variant
RPSSLPDF

SEQ ID NO: 1123 - protein, 8 aa, Artificial Sequence
  phosphopeptide or phosphopeptide variant
RPSSLPDM

SEQ ID NO: 1124 - protein, 9 aa, Artificial Sequence
  phosphopeptide or phosphopeptide variant
RPSRSSPGV

SEQ ID NO: 1125 - protein, 9 aa, Artificial Sequence
  phosphopeptide or phosphopeptide variant
RPSRSSPGF

SEQ ID NO: 1126 - protein, 9 aa, Artificial Sequence
  phosphopeptide or phosphopeptide variant
RPSRSSPGM

SEQ ID NO: 1127 - protein, 9 aa, Artificial Sequence
  phosphopeptide or phosphopeptide variant
RPSSPALYL

SEQ ID NO: 1128 - protein, 9 aa, Artificial Sequence
  phosphopeptide or phosphopeptide variant
RPSSPALYV

SEQ ID NO: 1129 - protein, 9 aa, Artificial Sequence
  phosphopeptide or phosphopeptide variant
RPSSPALYM

SEQ ID NO: 1130 - protein, 9 aa, Artificial Sequence
  phosphopeptide or phosphopeptide variant
RPTSRLNRV

SEQ ID NO: 1131 - protein, 9 aa, Artificial Sequence
  phosphopeptide or phosphopeptide variant
RPTSRLNRF

SEQ ID NO: 1132 - protein, 9 aa, Artificial Sequence
  phosphopeptide or phosphopeptide variant
RPTSRLNRM

SEQ ID NO: 1133 - protein, 9 aa, Artificial Sequence
  phosphopeptide or phosphopeptide variant
RPVSPFQEV

SEQ ID NO: 1134 - protein, 9 aa, Artificial Sequence
  phosphopeptide or phosphopeptide variant
RPVSPFQEF

SEQ ID NO: 1135 - protein, 9 aa, Artificial Sequence
  phosphopeptide or phosphopeptide variant
RPVSPFQEM

SEQ ID NO: 1136 - protein, 9 aa, Artificial Sequence
  phosphopeptide or phosphopeptide variant
RPVTPVSDV

SEQ ID NO: 1137 - protein, 9 aa, Artificial Sequence
  phosphopeptide or phosphopeptide variant
RPVTPVSDF

SEQ ID NO: 1138 - protein, 9 aa, Artificial Sequence
  phosphopeptide or phosphopeptide variant
RPVTPVSDM

SEQ ID NO: 1139 - protein, 9 aa, Artificial Sequence
  phosphopeptide or phosphopeptide variant
RPWSPAVSL

SEQ ID NO: 1140 - protein, 9 aa, Artificial Sequence
  phosphopeptide or phosphopeptide variant
RPWSPAVSV

SEQ ID NO: 1141 - protein, 9 aa, Artificial Sequence
  phosphopeptide or phosphopeptide variant
RPWSPAVSF

SEQ ID NO: 1142 - protein, 9 aa, Artificial Sequence
  phosphopeptide or phosphopeptide variant
RPWSPAVSM

SEQ ID NO: 1143 - protein, 10 aa, Artificial Sequence
  phosphopeptide or phosphopeptide variant
RPYSPPFFSV

SEQ ID NO: 1144 - protein, 10 aa, Artificial Sequence
  phosphopeptide or phosphopeptide variant
RPYSPPFFSF

SEQ ID NO: 1145 - protein, 10 aa, Artificial Sequence
  phosphopeptide or phosphopeptide variant
RPYSPPFFSM

SEQ ID NO: 1146 - protein, 9 aa, Artificial Sequence
  phosphopeptide or phosphopeptide variant
RRKSQVAEV

SEQ ID NO: 1147 - protein, 9 aa, Artificial Sequence
  phosphopeptide or phosphopeptide variant
RRKSQVAEF

SEQ ID NO: 1148 - protein, 9 aa, Artificial Sequence
  phosphopeptide or phosphopeptide variant
RRKSQVAEM

SEQ ID NO: 1149 - protein, 9 aa, Artificial Sequence
  phosphopeptide or phosphopeptide variant
RPKSQVAEL

SEQ ID NO: 1150 - protein, 9 aa, Artificial Sequence
  phosphopeptide or phosphopeptide variant
RPKSQVAEV

SEQ ID NO: 1151 - protein, 9 aa, Artificial Sequence
  phosphopeptide or phosphopeptide variant
RPKSQVAEF

SEQ ID NO: 1152 - protein, 9 aa, Artificial Sequence
  phosphopeptide or phosphopeptide variant
RPKSQVAEM

SEQ ID NO: 1153 - protein, 12 aa, Artificial Sequence
  phosphopeptide or phosphopeptide variant
RRSSLDAEIDSV

SEQ ID NO: 1154 - protein, 12 aa, Artificial Sequence
  phosphopeptide or phosphopeptide variant
RRSSLDAEIDSF

SEQ ID NO: 1155 - protein, 12 aa, Artificial Sequence
  phosphopeptide or phosphopeptide variant
RRSSLDAEIDSM

SEQ ID NO: 1156 - protein, 12 aa, Artificial Sequence
  phosphopeptide or phosphopeptide variant
RPSSLDAEIDSL

SEQ ID NO: 1157 - protein, 12 aa, Artificial Sequence
  phosphopeptide or phosphopeptide variant
RPSSLDAEIDSV

SEQ ID NO: 1158 - protein, 12 aa, Artificial Sequence
  phosphopeptide or phosphopeptide variant
RPSSLDAEIDSF

SEQ ID NO: 1159 - protein, 12 aa, Artificial Sequence
  phosphopeptide or phosphopeptide variant
RPSSLDAEIDSM

SEQ ID NO: 1160 - protein, 9 aa, Artificial Sequence
  phosphopeptide or phosphopeptide variant
RSESKDRKV

SEQ ID NO: 1161 - protein, 9 aa, Artificial Sequence
  phosphopeptide or phosphopeptide variant
RSESKDRKF

SEQ ID NO: 1162 - protein, 9 aa, Artificial Sequence
  phosphopeptide or phosphopeptide variant
RSESKDRKM

SEQ ID NO: 1163 - protein, 9 aa, Artificial Sequence
  phosphopeptide or phosphopeptide variant
RPESKDRKL

SEQ ID NO: 1164 - protein, 9 aa, Artificial Sequence
  phosphopeptide or phosphopeptide variant
RPESKDRKV

SEQ ID NO: 1165 - protein, 9 aa, Artificial Sequence
  phosphopeptide or phosphopeptide variant
RPESKDRKF

SEQ ID NO: 1166 - protein, 9 aa, Artificial Sequence
  phosphopeptide or phosphopeptide variant
RPESKDRKM

SEQ ID NO: 1167 - protein, 9 aa, Artificial Sequence
  phosphopeptide or phosphopeptide variant
RSGSLERKL

SEQ ID NO: 1168 - protein, 9 aa, Artificial Sequence
  phosphopeptide or phosphopeptide variant
RSGSLERKF

SEQ ID NO: 1169 - protein, 9 aa, Artificial Sequence
  phosphopeptide or phosphopeptide variant
RSGSLERKM

SEQ ID NO: 1170 - protein, 9 aa, Artificial Sequence
  phosphopeptide or phosphopeptide variant
RPGSLERKV

SEQ ID NO: 1171 - protein, 9 aa, Artificial Sequence
  phosphopeptide or phosphopeptide variant
RPGSLERKL

SEQ ID NO: 1172 - protein, 9 aa, Artificial Sequence
  phosphopeptide or phosphopeptide variant
RPGSLERKF

SEQ ID NO: 1173 - protein, 9 aa, Artificial Sequence
  phosphopeptide or phosphopeptide variant
RPGSLERKM

SEQ ID NO: 1174 - protein, 9 aa, Artificial Sequence
  phosphopeptide or phosphopeptide variant
RSLSPGGAL

SEQ ID NO: 1175 - protein, 9 aa, Artificial Sequence
  phosphopeptide or phosphopeptide variant
RSLSPGGAV

SEQ ID NO: 1176 - protein, 9 aa, Artificial Sequence
  phosphopeptide or phosphopeptide variant
RSLSPGGAF

SEQ ID NO: 1177 - protein, 9 aa, Artificial Sequence
  phosphopeptide or phosphopeptide variant
RSLSPGGAM

SEQ ID NO: 1178 - protein, 9 aa, Artificial Sequence
  phosphopeptide or phosphopeptide variant
RPLSPGGAL

SEQ ID NO: 1179 - protein, 9 aa, Artificial Sequence
  phosphopeptide or phosphopeptide variant
RPLSPGGAV

SEQ ID NO: 1180 - protein, 9 aa, Artificial Sequence
  phosphopeptide or phosphopeptide variant
RPLSPGGAF

SEQ ID NO: 1181 - protein, 9 aa, Artificial Sequence
  phosphopeptide or phosphopeptide variant
RPLSPGGAM

SEQ ID NO: 1182 - protein, 8 aa, Artificial Sequence
  phosphopeptide or phosphopeptide variant
RSLSPLLL

SEQ ID NO: 1183 - protein, 8 aa, Artificial Sequence
  phosphopeptide or phosphopeptide variant
RSLSPLLV

SEQ ID NO: 1184 - protein, 8 aa, Artificial Sequence
  phosphopeptide or phosphopeptide variant
RSLSPLLM

SEQ ID NO: 1185 - protein, 8 aa, Artificial Sequence
  phosphopeptide or phosphopeptide variant
RPLSPLLF

SEQ ID NO: 1186 - protein, 8 aa, Artificial Sequence
  phosphopeptide or phosphopeptide variant
RPLSPLLL

SEQ ID NO: 1187 - protein, 8 aa, Artificial Sequence
  phosphopeptide or phosphopeptide variant
RPLSPLLV

SEQ ID NO: 1188 - protein, 8 aa, Artificial Sequence
  phosphopeptide or phosphopeptide variant
RPLSPLLM

SEQ ID NO: 1189 - protein, 10 aa, Artificial Sequence
  phosphopeptide or phosphopeptide variant
RTESDSGLKL

SEQ ID NO: 1190 - protein, 10 aa, Artificial Sequence
  phosphopeptide or phosphopeptide variant
RTESDSGLKV

SEQ ID NO: 1191 - protein, 10 aa, Artificial Sequence
  phosphopeptide or phosphopeptide variant
RTESDSGLKF

SEQ ID NO: 1192 - protein, 10 aa, Artificial Sequence
  phosphopeptide or phosphopeptide variant
RTESDSGLKM

SEQ ID NO: 1193 - protein, 10 aa, Artificial Sequence
  phosphopeptide or phosphopeptide variant
RPESDSGLKL

SEQ ID NO: 1194 - protein, 10 aa, Artificial Sequence
  phosphopeptide or phosphopeptide variant
RPESDSGLKV

SEQ ID NO: 1195 - protein, 10 aa, Artificial Sequence
  phosphopeptide or phosphopeptide variant
RPESDSGLKF

SEQ ID NO: 1196 - protein, 10 aa, Artificial Sequence
  phosphopeptide or phosphopeptide variant
RPESDSGLKM

SEQ ID NO: 1197 - protein, 9 aa, Artificial Sequence
  phosphopeptide or phosphopeptide variant
RVRSPTRSL

SEQ ID NO: 1198 - protein, 9 aa, Artificial Sequence
  phosphopeptide or phosphopeptide variant
RVRSPTRSV

SEQ ID NO: 1199 - protein, 9 aa, Artificial Sequence
  phosphopeptide or phosphopeptide variant
RVRSPTRSF

SEQ ID NO: 1200 - protein, 9 aa, Artificial Sequence
  phosphopeptide or phosphopeptide variant
RVRSPTRSM

SEQ ID NO: 1201 - protein, 9 aa, Artificial Sequence
  phosphopeptide or phosphopeptide variant
RPRSPTRSL

SEQ ID NO: 1202 - protein, 9 aa, Artificial Sequence
  phosphopeptide or phosphopeptide variant
RPRSPTRSV

SEQ ID NO: 1203 - protein, 9 aa, Artificial Sequence
  phosphopeptide or phosphopeptide variant
RPRSPTRSF

SEQ ID NO: 1204 - protein, 9 aa, Artificial Sequence
  phosphopeptide or phosphopeptide variant
RPRSPTRSM

SEQ ID NO: 1205 - protein, 11 aa, Artificial Sequence
  phosphopeptide or phosphopeptide variant
SPEKAGRRSSV

SEQ ID NO: 1206 - protein, 11 aa, Artificial Sequence
  phosphopeptide or phosphopeptide variant
SPEKAGRRSSF

SEQ ID NO: 1207 - protein, 11 aa, Artificial Sequence
  phosphopeptide or phosphopeptide variant
SPEKAGRRSSM

SEQ ID NO: 1208 - protein, 10 aa, Artificial Sequence
  phosphopeptide or phosphopeptide variant
SPGLARKRSV

SEQ ID NO: 1209 - protein, 10 aa, Artificial Sequence
  phosphopeptide or phosphopeptide variant
SPGLARKRSF

SEQ ID NO: 1210 - protein, 10 aa, Artificial Sequence
  phosphopeptide or phosphopeptide variant
SPGLARKRSM

SEQ ID NO: 1211 - protein, 9 aa, Artificial Sequence
  phosphopeptide or phosphopeptide variant
SPKSPGLKL

SEQ ID NO: 1212 - protein, 9 aa, Artificial Sequence
  phosphopeptide or phosphopeptide variant
SPKSPGLKV

SEQ ID NO: 1213 - protein, 9 aa, Artificial Sequence
  phosphopeptide or phosphopeptide variant
SPKSPGLKF

SEQ ID NO: 1214 - protein, 9 aa, Artificial Sequence
  phosphopeptide or phosphopeptide variant
SPKSPGLKM

SEQ ID NO: 1215 - protein, 9 aa, Artificial Sequence
  phosphopeptide or phosphopeptide variant
SPKSPTAAV

SEQ ID NO: 1216 - protein, 9 aa, Artificial Sequence
  phosphopeptide or phosphopeptide variant
SPKSPTAAF

SEQ ID NO: 1217 - protein, 9 aa, Artificial Sequence
  phosphopeptide or phosphopeptide variant
SPKSPTAAM

SEQ ID NO: 1218 - protein, 9 aa, Artificial Sequence
  phosphopeptide or phosphopeptide variant
SPRSPGKPL

SEQ ID NO: 1219 - protein, 9 aa, Artificial Sequence
  phosphopeptide or phosphopeptide variant
SPRSPGKPV

SEQ ID NO: 1220 - protein, 9 aa, Artificial Sequence
  phosphopeptide or phosphopeptide variant
SPRSPGKPF

SEQ ID NO: 1221 - protein, 10 aa, Artificial Sequence
  phosphopeptide or phosphopeptide variant
SPRRSRSISV

SEQ ID NO: 1222 - protein, 10 aa, Artificial Sequence
  phosphopeptide or phosphopeptide variant
SPRRSRSISF

SEQ ID NO: 1223 - protein, 10 aa, Artificial Sequence
  phosphopeptide or phosphopeptide variant
SPRRSRSISM

SEQ ID NO: 1224 - protein, 11 aa, Artificial Sequence
  phosphopeptide or phosphopeptide variant
SPRTPVSPVKL

SEQ ID NO: 1225 - protein, 11 aa, Artificial Sequence
  phosphopeptide or phosphopeptide variant
SPRTPVSPVKV

SEQ ID NO: 1226 - protein, 11 aa, Artificial Sequence
  phosphopeptide or phosphopeptide variant
SPRTPVSPVKM

SEQ ID NO: 1227 - protein, 11 aa, Artificial Sequence
  phosphopeptide or phosphopeptide variant
SPSSPSVRRQV

SEQ ID NO: 1228 - protein, 11 aa, Artificial Sequence
  phosphopeptide or phosphopeptide variant
SPSSPSVRRQF

SEQ ID NO: 1229 - protein, 11 aa, Artificial Sequence
  phosphopeptide or phosphopeptide variant
SPSSPSVRRQM

SEQ ID NO: 1230 - protein, 13 aa, Artificial Sequence
  phosphopeptide or phosphopeptide variant
SPSTSRSGGSSRV

SEQ ID NO: 1231 - protein, 13 aa, Artificial Sequence
  phosphopeptide or phosphopeptide variant
SPSTSRSGGSSRF

SEQ ID NO: 1232 - protein, 13 aa, Artificial Sequence
  phosphopeptide or phosphopeptide variant
SPSTSRSGGSSRM

SEQ ID NO: 1233 - protein, 8 aa, Artificial Sequence
  phosphopeptide or phosphopeptide variant
SPVVHQSV

SEQ ID NO: 1234 - protein, 8 aa, Artificial Sequence
  phosphopeptide or phosphopeptide variant
SPVVHQSF

SEQ ID NO: 1235 - protein, 8 aa, Artificial Sequence
  phosphopeptide or phosphopeptide variant
SPVVHQSM

SEQ ID NO: 1236 - protein, 10 aa, Artificial Sequence
  phosphopeptide or phosphopeptide variant
TPAQPQRRSV

SEQ ID NO: 1237 - protein, 10 aa, Artificial Sequence
  phosphopeptide or phosphopeptide variant
TPAQPQRRSF

SEQ ID NO: 1238 - protein, 10 aa, Artificial Sequence
  phosphopeptide or phosphopeptide variant
TPAQPQRRSM

SEQ ID NO: 1239 - protein, 11 aa, Artificial Sequence
  phosphopeptide or phosphopeptide variant
TPISPGRASGF

SEQ ID NO: 1240 - protein, 11 aa, Artificial Sequence
  phosphopeptide or phosphopeptide variant
TPISPGRASGV

SEQ ID NO: 1241 - protein, 11 aa, Artificial Sequence
  phosphopeptide or phosphopeptide variant
TPISPGRASGL

SEQ ID NO: 1242 - protein, 9 aa, Artificial Sequence
  phosphopeptide or phosphopeptide variant
TPRSPPLGV

SEQ ID NO: 1243 - protein, 9 aa, Artificial Sequence
  phosphopeptide or phosphopeptide variant
TPRSPPLGF

SEQ ID NO: 1244 - protein, 9 aa, Artificial Sequence
  phosphopeptide or phosphopeptide variant
TPRSPPLGM

SEQ ID NO: 1245 - protein, 10 aa, Artificial Sequence
  phosphopeptide or phosphopeptide variant
TPRSPPLGLL

SEQ ID NO: 1246 - protein, 10 aa, Artificial Sequence
  phosphopeptide or phosphopeptide variant
TPRSPPLGLV

SEQ ID NO: 1247 - protein, 10 aa, Artificial Sequence
  phosphopeptide or phosphopeptide variant
TPRSPPLGLF

SEQ ID NO: 1248 - protein, 10 aa, Artificial Sequence
  phosphopeptide or phosphopeptide variant
TPRSPPLGLM

SEQ ID NO: 1249 - protein, 13 aa, Artificial Sequence
  phosphopeptide or phosphopeptide variant
VPRSPKHAHSSSV

SEQ ID NO: 1250 - protein, 13 aa, Artificial Sequence
  phosphopeptide or phosphopeptide variant
VPRSPKHAHSSSF

SEQ ID NO: 1251 - protein, 13 aa, Artificial Sequence
  phosphopeptide or phosphopeptide variant
VPRSPKHAHSSSM

SEQ ID NO: 1252 - protein, 11 aa, Artificial Sequence
  phosphopeptide or phosphopeptide variant
YQRSFDEVEGL

SEQ ID NO: 1253 - protein, 11 aa, Artificial Sequence
  phosphopeptide or phosphopeptide variant
YQRSFDEVEGF

SEQ ID NO: 1254 - protein, 11 aa, Artificial Sequence
  phosphopeptide or phosphopeptide variant
YQRSFDEVEGM

SEQ ID NO: 1255 - protein, 11 aa, Artificial Sequence
  phosphopeptide or phosphopeptide variant
YPRSFDEVEGV

SEQ ID NO: 1256 - protein, 11 aa, Artificial Sequence
  phosphopeptide or phosphopeptide variant
YPRSFDEVEGL

SEQ ID NO: 1257 - protein, 11 aa, Artificial Sequence
  phosphopeptide or phosphopeptide variant
YPRSFDEVEGF

SEQ ID NO: 1258 - protein, 11 aa, Artificial Sequence
  phosphopeptide or phosphopeptide variant
YPRSFDEVEGM

SEQ ID NO: 1259 - protein, 12 aa, Artificial Sequence
  phosphopeptide or phosphopeptide variant
YQRSFDEVEGVL

SEQ ID NO: 1260 - protein, 12 aa, Artificial Sequence
  phosphopeptide or phosphopeptide variant
YQRSFDEVEGVV

SEQ ID NO: 1261 - protein, 12 aa, Artificial Sequence
  phosphopeptide or phosphopeptide variant
YQRSFDEVEGVM

SEQ ID NO: 1262 - protein, 12 aa, Artificial Sequence
  phosphopeptide or phosphopeptide variant
YPRSFDEVEGVF

SEQ ID NO: 1263 - protein, 12 aa, Artificial Sequence
  phosphopeptide or phosphopeptide variant
YPRSFDEVEGVL

SEQ ID NO: 1264 - protein, 12 aa, Artificial Sequence
  phosphopeptide or phosphopeptide variant
YPRSFDEVEGVV

SEQ ID NO: 1265 - protein, 12 aa, Artificial Sequence
  phosphopeptide or phosphopeptide variant
YPRSFDEVEGVM

SEQ ID NO: 1266 - protein, 9 aa, Artificial Sequence
  phosphopeptide or phosphopeptide variant
AMAASPHAV

SEQ ID NO: 1267 - protein, 11 aa, Artificial Sequence
  phosphopeptide or phosphopeptide variant
AVVSPPALHNA

SEQ ID NO: 1268 - protein, 9 aa, Artificial Sequence
  phosphopeptide or phosphopeptide variant
GLLGSPVRA

SEQ ID NO: 1269 - protein, 10 aa, Artificial Sequence
  phosphopeptide or phosphopeptide variant
ILKSPEIQRA

SEQ ID NO: 1270 - protein, 9 aa, Artificial Sequence
  phosphopeptide or phosphopeptide variant
IMDRTPEKL

SEQ ID NO: 1271 - protein, 10 aa, Artificial Sequence
  phosphopeptide or phosphopeptide variant
KLASPELERL

SEQ ID NO: 1272 - protein, 10 aa, Artificial Sequence
  phosphopeptide or phosphopeptide variant
KLFPDTPLAL

SEQ ID NO: 1273 - protein, 11 aa, Artificial Sequence
  phosphopeptide or phosphopeptide variant
KLIDIVSSQKV

SEQ ID NO: 1274 - protein, 13 aa, Artificial Sequence
  phosphopeptide or phosphopeptide variant
KLLDFGSLSNLQV

SEQ ID NO: 1275 - protein, 10 aa, Artificial Sequence
  phosphopeptide or phosphopeptide variant
KLLSPSNEKL

SEQ ID NO: 1276 - protein, 10 aa, Artificial Sequence
  phosphopeptide or phosphopeptide variant
KLLSSAQRTL

SEQ ID NO: 1277 - protein, 11 aa, Artificial Sequence
  phosphopeptide or phosphopeptide variant
KLMSPKADVKL

SEQ ID NO: 1278 - protein, 9 aa, Artificial Sequence
  phosphopeptide or phosphopeptide variant
LMFSPVTSL

SEQ ID NO: 1279 - protein, 9 aa, Artificial Sequence
  phosphopeptide or phosphopeptide variant
RLDSYVRSL

SEQ ID NO: 1280 - protein, 9 aa, Artificial Sequence
  phosphopeptide or phosphopeptide variant
RLFSKELRC

SEQ ID NO: 1281 - protein, 9 aa, Artificial Sequence
  phosphopeptide or phosphopeptide variant
RLLSPLSSA

SEQ ID NO: 1282 - protein, 9 aa, Artificial Sequence
  phosphopeptide or phosphopeptide variant
RLQSTSERL

SEQ ID NO: 1283 - protein, 9 aa, Artificial Sequence
  phosphopeptide or phosphopeptide variant
RLSSPLHFV

SEQ ID NO: 1284 - protein, 11 aa, Artificial Sequence
  phosphopeptide or phosphopeptide variant
RQDSTPGKVFL

SEQ ID NO: 1285 - protein, 9 aa, Artificial Sequence
  phosphopeptide or phosphopeptide variant
RQISQDVKL

SEQ ID NO: 1286 - protein, 10 aa, Artificial Sequence
  phosphopeptide or phosphopeptide variant
RQLSSGVSEI

SEQ ID NO: 1287 - protein, 9 aa, Artificial Sequence
  phosphopeptide or phosphopeptide variant
RTFSPTYGL

SEQ ID NO: 1288 - protein, 9 aa, Artificial Sequence
  phosphopeptide or phosphopeptide variant
RTLSHISEA

SEQ ID NO: 1289 - protein, 9 aa, Artificial Sequence
  phosphopeptide or phosphopeptide variant
RVASPTSGV

SEQ ID NO: 1290 - protein, 9 aa, Artificial Sequence
  phosphopeptide or phosphopeptide variant
SLLTSPPKA

SEQ ID NO: 1291 - protein, 9 aa, Artificial Sequence
  phosphopeptide or phosphopeptide variant
SLQPRSHSV

SEQ ID NO: 1292 - protein, 9 aa, Artificial Sequence
  phosphopeptide or phosphopeptide variant
SMTRSPPRV

SEQ ID NO: 1293 - protein, 11 aa, Artificial Sequence
  phosphopeptide or phosphopeptide variant
TLASPSVFKST

SEQ ID NO: 1294 - protein, 9 aa, Artificial Sequence
  phosphopeptide or phosphopeptide variant
VLLSPVPEL

SEQ ID NO: 1295 - protein, 10 aa, Artificial Sequence
  phosphopeptide or phosphopeptide variant
VMFRTPLASV

SEQ ID NO: 1296 - protein, 9 aa, Artificial Sequence
  phosphopeptide or phosphopeptide variant
VMIGSPKKV

SEQ ID NO: 1297 - protein, 10 aa, Artificial Sequence
  phosphopeptide or phosphopeptide variant
YLDSGIHSGA

SEQ ID NO: 1298 - protein, 9 aa, Artificial Sequence
  phosphopeptide or phosphopeptide variant
ALAASPHAV

SEQ ID NO: 1299 - protein, 11 aa, Artificial Sequence
  phosphopeptide or phosphopeptide variant
AVVSPPALHNV

SEQ ID NO: 1300 - protein, 11 aa, Artificial Sequence
  phosphopeptide or phosphopeptide variant
ALVSPPALHNV

SEQ ID NO: 1301 - protein, 11 aa, Artificial Sequence
  phosphopeptide or phosphopeptide variant
ALVSPPALHNA

SEQ ID NO: 1302 - protein, 11 aa, Artificial Sequence
  phosphopeptide or phosphopeptide variant
AMVSPPALHNA

SEQ ID NO: 1303 - protein, 11 aa, Artificial Sequence
  phosphopeptide or phosphopeptide variant
AMVSPPALHNV

SEQ ID NO: 1304 - protein, 9 aa, Artificial Sequence
  phosphopeptide or phosphopeptide variant
GMLGSPVRV

SEQ ID NO: 1305 - protein, 10 aa, Artificial Sequence
  phosphopeptide or phosphopeptide variant
ILKSPEIQRV

SEQ ID NO: 1306 - protein, 10 aa, Artificial Sequence
  phosphopeptide or phosphopeptide variant
IMKSPEIQRV

SEQ ID NO: 1307 - protein, 10 aa, Artificial Sequence
  phosphopeptide or phosphopeptide variant
IMKSPEIQRA

SEQ ID NO: 1308 - protein, 9 aa, Artificial Sequence
  phosphopeptide or phosphopeptide variant
ILDRTPEKL

SEQ ID NO: 1309 - protein, 9 aa, Artificial Sequence
  phosphopeptide or phosphopeptide variant
IMDRTPEKV

SEQ ID NO: 1310 - protein, 9 aa, Artificial Sequence
  phosphopeptide or phosphopeptide variant
ILDRTPEKV

SEQ ID NO: 1311 - protein, 10 aa, Artificial Sequence
  phosphopeptide or phosphopeptide variant
KMASPELERL

SEQ ID NO: 1312 - protein, 10 aa, Artificial Sequence
  phosphopeptide or phosphopeptide variant
KLASPELERV

SEQ ID NO: 1313 - protein, 10 aa, Artificial Sequence
  phosphopeptide or phosphopeptide variant
KMASPELERV

SEQ ID NO: 1314 - protein, 10 aa, Artificial Sequence
  phosphopeptide or phosphopeptide variant
KMFPDTPLAL

SEQ ID NO: 1315 - protein, 10 aa, Artificial Sequence
  phosphopeptide or phosphopeptide variant
KLFPDTPLAV

SEQ ID NO: 1316 - protein, 10 aa, Artificial Sequence
  phosphopeptide or phosphopeptide variant
KMFPDTPLAV

SEQ ID NO: 1317 - protein, 10 aa, Artificial Sequence
  phosphopeptide or phosphopeptide variant
KMDIVSSQKV

SEQ ID NO: 1318 - protein, 10 aa, Artificial Sequence
  phosphopeptide or phosphopeptide variant
KLDIVSSQKV

SEQ ID NO: 1319 - protein, 13 aa, Artificial Sequence
  phosphopeptide or phosphopeptide variant
KMLDFGSLSNLQV

SEQ ID NO: 1320 - protein, 10 aa, Artificial Sequence
  phosphopeptide or phosphopeptide variant
KMLSPSNEKL

SEQ ID NO: 1321 - protein, 10 aa, Artificial Sequence
  phosphopeptide or phosphopeptide variant
KLLSPSNEKV

SEQ ID NO: 1322 - protein, 10 aa, Artificial Sequence
  phosphopeptide or phosphopeptide variant
KMLSPSNEKV

SEQ ID NO: 1323 - protein, 10 aa, Artificial Sequence
  phosphopeptide or phosphopeptide variant
KMLSSAQRTL

SEQ ID NO: 1324 - protein, 10 aa, Artificial Sequence
  phosphopeptide or phosphopeptide variant
KLLSSAQRTV

SEQ ID NO: 1325 - protein, 10 aa, Artificial Sequence
  phosphopeptide or phosphopeptide variant
KMLSSAQRTV

SEQ ID NO: 1326 - protein, 11 aa, Artificial Sequence
  phosphopeptide or phosphopeptide variant
KMMSPKADVKL

SEQ ID NO: 1327 - protein, 11 aa, Artificial Sequence
  phosphopeptide or phosphopeptide variant
KLMSPKADVKV

SEQ ID NO: 1328 - protein, 11 aa, Artificial Sequence
  phosphopeptide or phosphopeptide variant
KMMSPKADVKV

SEQ ID NO: 1329 - protein, 9 aa, Artificial Sequence
  phosphopeptide or phosphopeptide variant
LLFSPVTSL

SEQ ID NO: 1330 - protein, 9 aa, Artificial Sequence
  phosphopeptide or phosphopeptide variant
LMFSPVTSV

SEQ ID NO: 1331 - protein, 9 aa, Artificial Sequence
  phosphopeptide or phosphopeptide variant
LLFSPVTSV

SEQ ID NO: 1332 - protein, 9 aa, Artificial Sequence
  phosphopeptide or phosphopeptide variant
RMDSYVRSL

SEQ ID NO: 1333 - protein, 9 aa, Artificial Sequence
  phosphopeptide or phosphopeptide variant
RLDSYVRSV

SEQ ID NO: 1334 - protein, 9 aa, Artificial Sequence
  phosphopeptide or phosphopeptide variant
RMDSYVRSV

SEQ ID NO: 1335 - protein, 9 aa, Artificial Sequence
  phosphopeptide or phosphopeptide variant
RMFSKELRC

SEQ ID NO: 1336 - protein, 9 aa, Artificial Sequence
  phosphopeptide or phosphopeptide variant
RLFSKELRV

SEQ ID NO: 1337 - protein, 9 aa, Artificial Sequence
  phosphopeptide or phosphopeptide variant
RMFSKELRV

SEQ ID NO: 1338 - protein, 9 aa, Artificial Sequence
  phosphopeptide or phosphopeptide variant
RMLSPLSSA

SEQ ID NO: 1339 - protein, 9 aa, Artificial Sequence
  phosphopeptide or phosphopeptide variant
RLLSPLSSV

SEQ ID NO: 1340 - protein, 9 aa, Artificial Sequence
  phosphopeptide or phosphopeptide variant
RMLSPLSSV

SEQ ID NO: 1341 - protein, 9 aa, Artificial Sequence
  phosphopeptide or phosphopeptide variant
RMQSTSERL

SEQ ID NO: 1342 - protein, 9 aa, Artificial Sequence
  phosphopeptide or phosphopeptide variant
RLQSTSERV

SEQ ID NO: 1343 - protein, 9 aa, Artificial Sequence
  phosphopeptide or phosphopeptide variant
RMQSTSERV

SEQ ID NO: 1344 - protein, 9 aa, Artificial Sequence
  phosphopeptide or phosphopeptide variant
RMSSPLHFV

SEQ ID NO: 1345 - protein, 11 aa, Artificial Sequence
  phosphopeptide or phosphopeptide variant
RLDSTPGKVFL

SEQ ID NO: 1346 - protein, 11 aa, Artificial Sequence
  phosphopeptide or phosphopeptide variant
RMDSTPGKVFL

SEQ ID NO: 1347 - protein, 11 aa, Artificial Sequence
  phosphopeptide or phosphopeptide variant
RQDSTPGKVFV

SEQ ID NO: 1348 - protein, 11 aa, Artificial Sequence
  phosphopeptide or phosphopeptide variant
RMDSTPGKVFV

SEQ ID NO: 1349 - protein, 11 aa, Artificial Sequence
  phosphopeptide or phosphopeptide variant
RLDSTPGKVFV

SEQ ID NO: 1350 - protein, 9 aa, Artificial Sequence
  phosphopeptide or phosphopeptide variant
RLISQDVKL

SEQ ID NO: 1351 - protein, 9 aa, Artificial Sequence
  phosphopeptide or phosphopeptide variant
RMISQDVKL

SEQ ID NO: 1352 - protein, 9 aa, Artificial Sequence
  phosphopeptide or phosphopeptide variant
RQISQDVKV

SEQ ID NO: 1353 - protein, 9 aa, Artificial Sequence
  phosphopeptide or phosphopeptide variant
RLISQDVKV

SEQ ID NO: 1354 - protein, 9 aa, Artificial Sequence
  phosphopeptide or phosphopeptide variant
RMISQDVKV

SEQ ID NO: 1355 - protein, 10 aa, Artificial Sequence
  phosphopeptide or phosphopeptide variant
RLLSSGVSEI

SEQ ID NO: 1356 - protein, 10 aa, Artificial Sequence
  phosphopeptide or phosphopeptide variant
RMLSSGVSEI

SEQ ID NO: 1357 - protein, 10 aa, Artificial Sequence
  phosphopeptide or phosphopeptide variant
RQLSSGVSEV

SEQ ID NO: 1358 - protein, 10 aa, Artificial Sequence
  phosphopeptide or phosphopeptide variant
RLLSSGVSEV

SEQ ID NO: 1359 - protein, 10 aa, Artificial Sequence
  phosphopeptide or phosphopeptide variant
RMLSSGVSEV

SEQ ID NO: 1360 - protein, 9 aa, Artificial Sequence
  phosphopeptide or phosphopeptide variant
RLFSPTYGL

SEQ ID NO: 1361 - protein, 9 aa, Artificial Sequence
  phosphopeptide or phosphopeptide variant
RMFSPTYGL

SEQ ID NO: 1362 - protein, 9 aa, Artificial Sequence
  phosphopeptide or phosphopeptide variant
RTFSPTYGV

SEQ ID NO: 1363 - protein, 9 aa, Artificial Sequence
  phosphopeptide or phosphopeptide variant
RLFSPTYGV

SEQ ID NO: 1364 - protein, 9 aa, Artificial Sequence
  phosphopeptide or phosphopeptide variant
RMFSPTYGV

SEQ ID NO: 1365 - protein, 9 aa, Artificial Sequence
  phosphopeptide or phosphopeptide variant
RLLSHISEA

SEQ ID NO: 1366 - protein, 9 aa, Artificial Sequence
  phosphopeptide or phosphopeptide variant
RMLSHISEA

SEQ ID NO: 1367 - protein, 9 aa, Artificial Sequence
  phosphopeptide or phosphopeptide variant
RTLSHISEV

SEQ ID NO: 1368 - protein, 9 aa, Artificial Sequence
  phosphopeptide or phosphopeptide variant
RLLSHISEV

SEQ ID NO: 1369 - protein, 9 aa, Artificial Sequence
  phosphopeptide or phosphopeptide variant
RMLSHISEV

SEQ ID NO: 1370 - protein, 9 aa, Artificial Sequence
  phosphopeptide or phosphopeptide variant
RLASPTSGV

SEQ ID NO: 1371 - protein, 9 aa, Artificial Sequence
  phosphopeptide or phosphopeptide variant
RMASPTSGV

SEQ ID NO: 1372 - protein, 9 aa, Artificial Sequence
  phosphopeptide or phosphopeptide variant
SMLTSPPKA

SEQ ID NO: 1373 - protein, 9 aa, Artificial Sequence
  phosphopeptide or phosphopeptide variant
SLLTSPPKV

SEQ ID NO: 1374 - protein, 9 aa, Artificial Sequence
  phosphopeptide or phosphopeptide variant
SMLTSPPKV

SEQ ID NO: 1375 - protein, 9 aa, Artificial Sequence
  phosphopeptide or phosphopeptide variant
SMQPRSHSV

SEQ ID NO: 1376 - protein, 9 aa, Artificial Sequence
  phosphopeptide or phosphopeptide variant
SMTRSPPRV

SEQ ID NO: 1377 - protein, 11 aa, Artificial Sequence
  phosphopeptide or phosphopeptide variant
TMASPSVFKST

SEQ ID NO: 1378 - protein, 11 aa, Artificial Sequence
  phosphopeptide or phosphopeptide variant
TLASPSVFKSV

SEQ ID NO: 1379 - protein, 11 aa, Artificial Sequence
  phosphopeptide or phosphopeptide variant
TMASPSVFKSV

SEQ ID NO: 1380 - protein, 9 aa, Artificial Sequence
  phosphopeptide or phosphopeptide variant
VMLSPVPEL

SEQ ID NO: 1381 - protein, 9 aa, Artificial Sequence
  phosphopeptide or phosphopeptide variant
VLLSPVPEV

SEQ ID NO: 1382 - protein, 9 aa, Artificial Sequence
  phosphopeptide or phosphopeptide variant
VMLSPVPEV

SEQ ID NO: 1383 - protein, 10 aa, Artificial Sequence
  phosphopeptide or phosphopeptide variant
VLFRTPLASV

SEQ ID NO: 1384 - protein, 9 aa, Artificial Sequence
  phosphopeptide or phosphopeptide variant
VLIGSPKKV

SEQ ID NO: 1385 - protein, 10 aa, Artificial Sequence
  phosphopeptide or phosphopeptide variant
YMDSGIHSGA

SEQ ID NO: 1386 - protein, 10 aa, Artificial Sequence
  phosphopeptide or phosphopeptide variant
YMDSGIHSGV

SEQ ID NO: 1387 - protein, 10 aa, Artificial Sequence
  phosphopeptide or phosphopeptide variant
YLDSGIHSGV

SEQ ID NO: 1388 - protein, 10 aa, Artificial Sequence
  phosphopeptide or phosphopeptide variant
YLDSGIHSGA

SEQ ID NO: 1389 - protein, 10 aa, Artificial Sequence
  phosphopeptide or phosphopeptide variant
YMDSGIHSGA

SEQ ID NO: 1390 - protein, 10 aa, Artificial Sequence
  phosphopeptide or phosphopeptide variant
YMDSGIHSGV

SEQ ID NO: 1391 - protein, 10 aa, Artificial Sequence
  phosphopeptide or phosphopeptide variant
YLDSGIHSGV

(19) United States
(12) Patent Application Publication
(10) Pub. No.: US 2018/0066017 A1
(43) Pub. Date: Mar. 8, 2018

(54) CLASS I MHC PHOSPHOPEPTIDES FOR CANCER IMMUNOTHERAPY AND DIAGNOSIS

(71) Applicant: Agenus Inc., Lexington, MA (US)

(72) Inventors: Donald F. Hunt, Charlottesville, VA (US); Jeffrey Shabanowitz, Charlottesville, VA (US); Jennifer Cottine, Charlottesville, VA (US); Ann M. English, Palmyra, VA (US); Andrew Norris, Palmyra, VA (US); Victor H. Engelhard, Crozet, VA (US); Mark Cobbold, Birmingham (GB); Kara L. Cummings, Charlottesville, VA (US); Angela Zarling, Charlottesville, VA (US); Rebecca C. Obeng, Charlottesville, VA (US); Jie Qian, Somerset, NJ (US)

(21) Appl. No.: 15/483,274

(22) Filed: Apr. 10, 2017

Related U.S. Application Data
(63) Continuation of application No. 13/699,563, filed on Jun. 13, 2013, now abandoned.
(60) Provisional application No. 61/347,559, filed on May 24, 2010.

(51) Int. Cl. C07K 7/06; G01N 33/68; C07K 16/18; C07K 7/08; A61K 39/00

(52) U.S. Cl. CPC C07K 7/06; G01N 33/6893; C07K 16/18; A61K 38/00; A61K 39/0011; C07K 7/08

(57) ABSTRACT

A set of phosphorylated peptides are presented by HLA A*0101, A*0201, A*0301, B*4402, B*2705, B*1402, and B*0702 on the surface of melanoma cells. They have the potential to (a) stimulate an immune response to the cancer, (b) to function as immunotherapeutics in adoptive T-cell therapy or as a vaccine, (c) to facilitate antibody recognition of the tumor boundaries in surgical pathology samples, and (d) act as biomarkers for early detection of the disease. Phosphorylated peptides are also presented for other cancers.

### CROSS-REFERENCE TO RELATED APPLICATIONS

This application is a continuation of U.S. patent application Ser. No. 13/699,563, filed Jun. 13, 2013, which is a 35 U.S.C. § 371 filing of International Patent Application No. PCT/US11/37699, filed May 24, 2011, which claims the benefit of U.S. Provisional Patent Application Ser. No. 61/347,559, filed May 24, 2010, the entire disclosures of which are hereby incorporated herein by reference.

### STATEMENT REGARDING FEDERALLY SPONSORED RESEARCH OR DEVELOPMENT

This invention was made with government support under R01 AI20963 and AI33993 awarded by the National Institutes of Health. The government has certain rights in the invention.

### TECHNICAL FIELD OF THE INVENTION

This invention is related to the area of cancer diagnostics, and therapeutics. In particular, it relates to immunological reactions mediated through MHC class I molecules.

### BACKGROUND OF THE INVENTION

The mammalian immune system has evolved a variety of mechanisms to protect the host from cancerous cells. An important component of this response is mediated by cells referred to as T cells. Cytotoxic T lymphocytes (CTL) are specialized T cells that primarily function by recognizing and killing cancerous cells or infected cells, but they can also function by secreting soluble molecules referred to as cytokines that can mediate a variety of effects on the immune system. T helper cells primarily function by recognizing antigen on specialized antigen presenting cells, and in turn secreting cytokines that activate B cells, T cells, and macrophages. A variety of evidence suggests that immunotherapy designed to stimulate a tumor-specific CTL response would be effective in controlling cancer. For example, it has been shown that human CTL recognize sarcomas (Slovin et al., 1986, J Immunol 137, 3042-3048), renal cell carcinomas (Schendel et al., 1993, J Immunol 151, 4209-4220), colorectal carcinomas (Jacob et al., 1997, Int J Cancer 71, 325-332), ovarian carcinomas (Peoples et al., 1993, Surgery 114, 227-234), pancreatic carcinomas (Peiper et al., 1997, Eur J Immunol 27, 1115-1123), squamous tumors of the head and neck (Yasumura et al., 1993, Cancer Res 53, 1461-1468), and squamous carcinomas of the lung (Slingluff et al., 1994, Cancer Res 54, 2731-2737; Yoshino et al., 1994, Cancer Res 54, 3387-3390). The largest number of reports of human tumor-reactive CTLs, however, has concerned melanomas (Boon et al., 1994, Annu Rev Immunol 12, 337-365). The ability of tumor-specific CTL to mediate tumor regression, in both human (Parmiani et al., 2002, J Natl Cancer Inst 94, 805-818; Weber, 2002, Cancer Invest 20, 208-221) and animal models, suggests that methods directed at increasing CTL activity would likely have a beneficial effect with respect to tumor treatment.

Melanoma, or skin cancer, is a disease that is diagnosed in approximately 54,200 persons per year. Conventional therapy for the disease includes surgery, radiation therapy, and chemotherapy. In spite of these approaches to treatment, approximately 7,600 individuals die in the United States every year due to melanoma. Overall, the 5-year survival rate for the disease is 88%. The survival rate drops, however, in more advanced stages of the disease with only about 50% of Stage III patients, and 20-30% of Stage IV patients surviving past five years. In patients where the melanoma has metastasized to distant sites, the 5-year survival dips to only 12%. Clearly, there is a population of melanoma patients that is in need of better treatment options. More recently, in an attempt to decrease the number of deaths attributed to melanoma, immunotherapy has been added to the arsenal of treatments used against the disease.

In order for CTL to kill or secrete cytokines in response to a cancer cell, the CTL must first recognize the cancer cell (Townsend and Bodmer, 1989). This process involves the interaction of the T cell receptor, located on the surface of the CTL, with what is generically referred to as an MHC-peptide complex which is located on the surface of the cancerous cell. MHC (major histocompatibility-complex)-encoded molecules have been subdivided into two types, and are referred to as class I and class II MHC-encoded molecules. In the human immune system, MHC molecules are referred to as human leukocyte antigens (HLA). Within the MHC complex, located on chromosome six, are three different loci that encode for class I MHC molecules. MHC molecules encoded at these loci are referred to as HLA-A, HLA-B, and HLA-C. The genes that can be encoded at each of these loci are extremely polymorphic, and thus, different individuals within the population express different class I MHC molecules on the surface of their cells. HLA-A1, HLA-A2, HLA-A3, HLA-B7, HLA-B14, HLA-B27, and HLA-B44 are examples of different class I MHC molecules that can be expressed from these loci.

The peptides which associate with the MI-IC molecules can either be derived from proteins made within the cell, in which case they typically associate with class I MHC molecules (Rock and Goldberg, 1999, Annu Rev Immunol 17, 739-779); or they can be derived from proteins which are acquired from outside of the cell, in which case they typically associate with class II MHC molecules (Watts, 1997, Annu Rev Immunol 15, 821-850). The peptides that evoke a cancer-specific CTL response most typically associate with class I MHC molecules. The peptides themselves are typically nine amino acids in length, but can vary from a minimum length of eight amino acids to a maximum of fourteen amino acids in length. Tumor antigens may also bind to class II MHC molecules on antigen presenting cells and provoke a T helper cell response. The peptides that bind to class II MHC molecules are generally twelve to nineteen amino acids in length, but can be as short as ten amino acids and as long as thirty amino acids.

The process by which intact proteins are degraded into peptides is referred to as antigen processing. Two major pathways of antigen processing occur within cells (Rock and Goldberg, 1999, Annu Rev Immunol 17, 739-779). One pathway, which is largely restricted to professional antigen presenting cells such as dendritic cells, macrophages, and B cells, degrades proteins that are typically phagocytosed or endocytosed into the cell. Peptides derived from this pathway can be presented on either class I or to class II MHC molecules. A second pathway of antigen processing is present in essentially all cells of the body. This second pathway primarily degrades proteins that are made within the cells, and the peptides derived from this pathway primarily bind to class I MHC molecules. Antigen processing by this latter pathway involves polypeptide synthesis and proteolysis in the cytoplasm, followed by transport of peptides to the plasma membrane for presentation. These peptides, initially being transported into the endoplasmic reticulum of the cell, become associated with newly synthesized class I MHC molecules and the resulting complexes are then transported to the cell surface. Peptides derived from membrane and secreted proteins have also been identified. In some cases these peptides correspond to the signal sequence of the proteins which is cleaved from the protein by the signal peptidase. In other cases, it is thought that some fraction of the membrane and secreted proteins are transported from the endoplasmic reticulum into the cytoplasm where processing subsequently occurs. Once bound to the class I MHC molecule, the peptides are recognized by antigen-specific receptors on CTL. Several methods have been developed to identify the peptides recognized by CTL, each method of which relies on the ability of a CTL to recognize and kill only those cells expressing the appropriate class I MHC molecule with the peptide bound to it. Mere expression of the class I MHC molecule is insufficient to trigger the CTL to kill the target cell if the antigenic peptide is not bound to the class I MHC molecule. Such peptides can be derived from a non-self source, such as a pathogen (for example, following the infection of a cell by a bacterium or a virus) or from a self-derived protein within a cell, such as a cancerous cell. The tumor antigens from which the peptides are derived can broadly be categorized as differentiation antigens, cancer/testis antigens, mutated gene products, widely expressed proteins, viral antigens and most recently, phosphopeptides derived from dysregulated signal transduction pathways. (Zarling et al., PNAS 103, 12889-14894, 2006). Immunization with melanoma-derived, class I or class II MHC-encoded molecule associated peptides, or with a precursor polypeptide or protein that contains the peptide, or with a gene that encodes a polypeptide or protein containing the peptide, are forms of immunotherapy that can be employed in the treatment of melanoma. Identification of the immunogens is a necessary first step in the formulation of the appropriate immunotherapeutic agent or agents. Although a large number of tumor-associated peptide antigens recognized by tumor reactive CTL, have been identified, there are few examples of antigens that are derived from proteins that are selectively expressed on a broad array of tumors, as well as associated with cellular proliferation and/or transformation. Attractive candidates for this type of antigen are peptides derived from proteins that are differentially phosphorylated on serine (Ser), threonine (Thr), and tyrosine (Tyr) (Zarling et al., 2000, J Exp Med 192 1755-1762). Due to the increased and dysregulated phosphorylation of cellular proteins in transformed cells as compared to normal cells, tumors are likely to present a unique subset of phosphorylated peptides on the cell surface that are available for recognition by cytotoxic T-lymphocytes (CTL). Presently, there is no way to predict which protein phosphorylation sites in a cell will be unique to tumors, survive the antigen processing pathway, and be presented to the immune system in the context of 8-14 residue phosphopeptides bound to class I MHC molecules.

Thirty-six phosphopeptides were disclosed as presented in association with HLA A*0201 on cancer cells. Zarling, et al., Proc. Natl. Acad. Sciences, 103, 1488914894, 2006, Table I. Parent proteins for four of these peptides (β-catenin, insulin receptor substrate-2 (IRS-2), tensin-3 and Jun-C/D) are known to be associated with cytoplasmic signaling pathways and cellular transformation. While both normal and cancer cells lines express the parent proteins, only the three cancer lines express phosphorylated class I peptide sequences within IRS-2 and β-catenin, respectively.

Mice expressing a transgenic recombinant human A*0201 MHC molecule were immunized with a synthetic class I phosphopeptides from IRS-2 and β-catenin that were pulsed onto activated bone-marrow derived dendritic cells. Cytotoxic T-cells were generated that recognized all three cancer cell lines but not the control JY cells. Class I phosphopeptides from IRS-2 and β-catenin are highly immunogenic and are likely candidates for immunotherapy directed toward melanoma and ovarian cancer.

Adoptive T-cell therapy of melanoma is described in two recent publications. Dudley et al., J. Clin. Oncology 2008, 26: 5233-5239 and Rosenberg, Curr. Opinion in Immun 2009, 21: 233-240. For adoptive T-cell therapy, late stage metastatic melanoma patients are treated as if they were undergoing an organ transplant operation. Tumor is resected and cytotoxic T-cells that have infiltrated the tumor are harvested and exposed to a particular class I peptide antigen (MART -1). Those that recognize this antigen are then allowed to expand until the total number of MART-1 specific cells reach 100 billion. The patient receives whole body irradiation and chemotherapy to wipe out 98% of his/her immune system. The MART specific T-cells are then given back to the patient and circulate throughout the body looking for tumor. In the most recent clinical trial, tumors in 72% of the patients showed objective responses with this therapy at all sites of metastasis including lymph nodes, bone, lung, liver, and brain. Twenty-eight percent of the patients had complete remission of the disease.

There is a need in the art for additional class I phosphopeptide antigens to permit adoptive T-cell therapy to be extended to cancer patients that may not express the HLA-A*0201 allele, as well as new phosphopeptides for patients that express the HLA*0201 allele. There is a need in the art to treat a variety of other cancers by the same approach.

### SUMMARY OF THE INVENTION

One aspect of the invention is an isolated and purified phosphopeptide that consists of between 8 and 50 contiguous amino acid residues derived from a native human protein. The phosphopeptide comprises a sequence selected from SEQ ID NO: 1-1391 in which at least one serine, threonine, or tyrosine residue in the selected sequence is phosphorylated with a hydrolyzable or non-hydrolyzable phosphate group. Contiguous amino acids adjacent to the selected sequence in the phosphopeptide are selected from the adjacent residues in the native human protein. When the sequence is selected from SEQ ID NO: 1266-1297, the phosphopeptide is phosphorylated with a non-hydrolyzable phosphate group.

Another aspect of the invention is a method of immunizing a mammal to diminish the risk of, the growth of, or the invasiveness of a melanoma. A composition is administered to the mammal that activates CD8⁺ T cells. The composition comprises a phosphopeptide that consists of between 8 and 50 contiguous amino acid residues derived from a native human protein. The phosphopeptide comprises a sequence selected from SEQ ID NO: 1-1391 in which at least one serine, threonine, or tyrosine residue in the selected sequence is phosphorylated with a hydrolyzable or non-hydrolyzable phosphate group. Contiguous amino acids adjacent to the selected sequence in the phosphopeptide are selected from the adjacent residues in the native human protein. When the sequence is selected from SEQ ID NO: 1266-1297, the phosphopeptide is phosphorylated with a non-hydrolyzable phosphate group.

Another aspect of the invention is a method that can be used for monitoring, diagnosis, or prognosis. A sample isolated from a patient is contacted with an antibody that specifically binds to a phosphopeptide. The phosphopeptide consists of between 8 and 50 contiguous amino acid residues derived from a native human protein. The phosphopeptide comprises a sequence selected from. SEQ ID NO: 1-1391 in which at least one serine, threonine, or tyrosine residue in the selected sequence is phosphorylated with a hydrolyzable or non-hydrolyzable phosphate group. Contiguous amino acids adjacent to the selected sequence in the phosphopeptide are selected from the adjacent residues in the native human protein. The antibody does not bind to a peptide consisting of the same amino acid sequence but devoid of phosphorylation. Antibody bound to the sample is measured or detected.

Still another aspect of the invention is a molecule that comprises an antigen-binding region of an antibody. The molecule specifically binds to a phosphopeptide and does not bind to a peptide consisting of the same amino acid sequence but devoid of phosphorylation. The phosphopeptide consists of between 8 and 50 contiguous amino acid residues derived from a native human protein. The phosphopeptide comprises a sequence selected from SEQ ID NO: 1-1391 in which at least one serine, threonine, or tyrosine residue in the selected sequence is phosphorylated with a hydrolyzable or non-hydrolyzable phosphate group. Contiguous amino acids adjacent to the selected sequence in the phosphopeptide are selected from the adjacent residues in the native human protein.

Still another aspect of the invention is a kit for measuring a phosphoprotein consisting of between 8 and 50 contiguous amino acids. The phosphoprotein comprises a sequence selected from SEQ ID NO: 1-1391 that includes a phosphorylated serine, threonine, or tyrosine residue. The kit comprises a molecule comprising an antigen-binding region of an antibody, wherein the molecule specifically binds to the phosphoprotein and does not bind to a protein consisting of the same amino acid sequence but devoid of phosphorylation.

Yet another aspect of the invention is a method, useful for producing an immunotherapeutic agent or tool. Dendritic cells are contacted in vitro with an isolated phosphopeptide consisting of between 8 and 50 contiguous amino acids. The phosphopeptide comprises a sequence selected from SEQ ID NO: 1-1391 which includes at least one serine, threonine, or tyrosine residue that is phosphorylated. The dendritic cells thereby become phosphopeptide-loaded. When the sequence is selected from SEQ ID NO:1266-1297, the phosphopeptide is phosphorylated with a non-hydrolyzable phosphate group. The dendritic cells made by the method provides an in vitro compositions of dendritic cells, useful as an immunotherapeutic agent.

A further aspect of the invention is a synthetic phosphopeptide comprising from 1050 amino acid residues, comprising the sequences, RVAsPTSGVK (SEQ ID NO: 53) or RVAsPTSGVKR (SEQ ID NO: 54), wherein the serine residue at position 4 is phosphorylated with a hydrolyzable or nonhydrolyzable phosphate group, and wherein adjacent amino acid residues to the sequence are adjacent sequences in the human insulin substrate-2 (IRS-2) protein. The phosphopeptide is useful for loading dendritic cells so that they present phosphopeptide on HLA-A*0301 molecules.

These and other aspects and embodiments which will be apparent to those of skill in the art upon reading the specification provide the art with immunological tools and agents useful for diagnosing, prognosing, monitoring, and treating human cancers.

### BRIEF DESCRIPTION OF THE DRAWINGS

FIGS. 1A and 1B are a graphic illustration of the recognition of naturally processed and presented phosphorylated peptides on cancer cells by the phosphopeptide-specific CTL. Phosphopeptide-specific CTL were incubated with the following cancer cell lines or EBV-transformed B lymphoblastoid cell lines (BLCL): COV413.AAD.A4 ovarian carcinoma, DM331.AAD.A4 and SLM2.AAD.A1 melanomas, MCF7.AAD.A2 and MDAMB231.AAD breast carcinomas, and JY EBV-BLCL. Supernatants were harvested and evaluated for the presence of murine IFNγ (produced by murine CTL lines). As a positive control, cancer cells were pulsed with the specific phosphopeptide to show that they are capable of presenting exogenously added peptide. In FIG. 1A, two phosphopeptide-specific CTL cell lines, 6850 and 6960 that are specific for the phosphopeptide GLLGpSPVRA (SEQ ID NO: 1268), recognize the phosphopeptide on all the cancer cell lines, but not the control cell line. In FIG. 1B, two phosphopeptide-specific CTL cell lines, 5183 and 63 that are specific for the phosphopeptide RVApSPTSGV (SEQ ID NO: 1289), recognize the phosphopeptide on all the cancer cell lines, but not the control cell line. The designation “pS” denotes a phosphoserine residue. The ordinate indicates murine IFNγ in pg/ml. The abscissa indicates each cell line.

FIGS. 2A-7D present Tables 2A-2F. FIGS. 2A-2D show melanoma HLA A*0301 phosphopeptides, A*0101 phosphopeptides, B*4402 phosphopeptides, B*2705 phosphopeptides, and B*1402 phosphopeptides. FIGS. 3A-3B show melanoma and/or leukemia HLA B*0702 phosphopeptides. FIGS. 4A-4S show melanoma HLA A*0301 phosphopeptides, A*0101 phosphopeptides, B*4402 phosphopeptides, B*2705 phosphopeptides, and B*1402 phosphopeptides and their sequence variants. FIGS. 5A-5H show melanoma and/or leukemia HLA B*0702 phosphopeptides and their sequence variants. FIG. 6 shows melanoma HLA-A*0201 phosphopeptides. FIGS. 7A-7D show melanoma HLA-A*0201 phosphopeptides and their sequence variants.

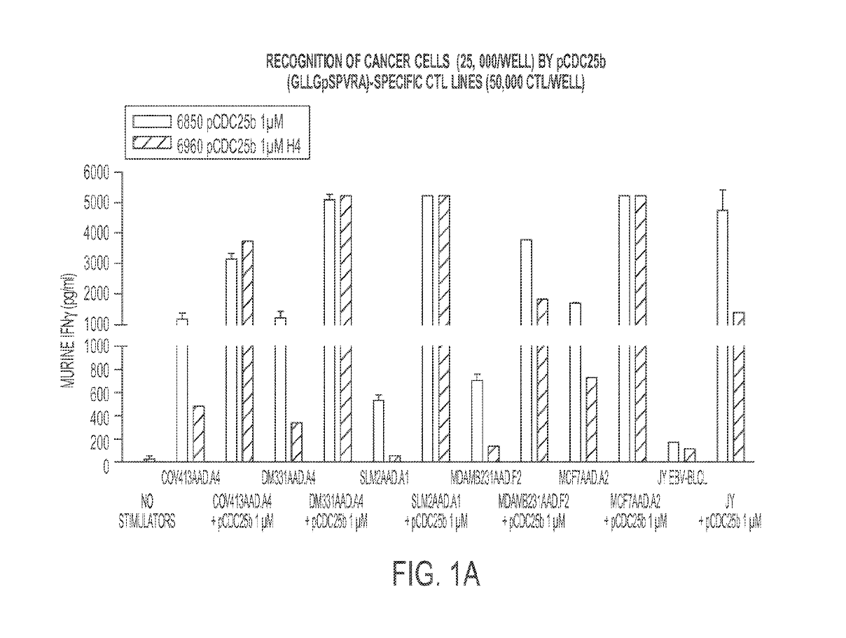

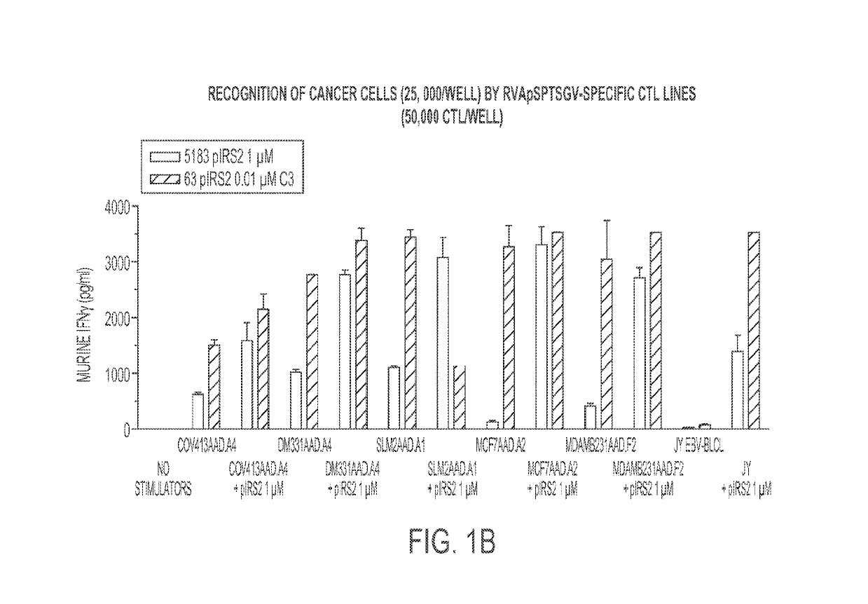

### DETAILED DESCRIPTION OF THE INVENTION

We have identified MHC class I phosphopeptides for use in diagnostics, immunotherapeutics, and adoptive T-cell therapy of melanoma patients. We provide over 200 class I MHC peptides presented on the surface of cancer cells in association with the HLA molecules A*0101 (SEQ ID NO: 70-97), A*0301 (SEQ ID NO: 1-69), and B*4402 (SEQ ID NO: 98-110), B*2705 (SEQ ID NO: 111-162), B*1402 (SEQ ID NO: 163-164), and B*0702 (SEQ ID NO: 165-246). Variants and mimetics of these peptides and of additional class I MHC phosphopeptides are also provided.

Although individuals in the human population display hundreds of different HLA alleles, some are more prevalent than others. For example, 88% of melanoma patients carry at least one of the six HLA alleles: HLA-A*0201 (29%), HLA-A*0101 (15%), HLA-A*0301 (14%), HLA-B*4402 (15%), HLA-B*0702 (12%), and HLA-B*-2705 (3%). One of our aims is to provide multiple phosphopeptides presented by each of the six most prevalent alleles and to use them as a cocktail, to optimize coverage of the human population and to minimize the possibility that the tumor will be able to escape immune surveillance by down-regulating expression of any one class I phosphopeptide.

Phosphopeptides of the invention are not the entire proteins from which they are derived. They are from 8 to 50 contiguous amino acid residues of the native human protein. They contain at least one of the MHC class I binding peptides listed in SEQ ID NO: 1-1391. Moreover, at least one of the serine, threonine, or tyrosine residues within the recited sequence is phosphorylated. The phosphorylation may be with a natural phosphorylation (—CH₂—O—PO₃H) or with an enzyme non-degradable, modified phosphorylation, such as (—CH₂—CF₂—PO₃H or —CH₂—CH₂—PO₃H). In certain specified positions, a native amino acid residue in a native human protein may be altered to enhance the binding to the MHC class I molecule. These occur in “anchor” positions of the phosphopeptides, often in positions 1, 2, 3, 9, or 10. Valine, alanine, lysine, leucine tyrosine, arginine, phenylalanine, proline, glutamic acid, threonine, serine, aspartic acid, tryptophan, and methionine may also be used as improved anchoring residues. Anchor residues for different HLA molecules are shown in Table 1. Some phosphopeptides may contain more than one of the peptides listed in SEQ ID NO: 1-1391, for example, if they are overlapping, adjacent, or nearby within the native protein from which they are derived. Phosphopeptides can also be mixed together to form a cocktail. The phosphopeptides may be in an admixture, or they may be linked together in a concatamer as a single molecule. Linkers between individual phosphopeptides may be used; these may, for example, be formed by any 10 to 20 amino acid residues. The linkers may be random sequences, or they may be optimized for degradation by dendritic cells.

TABLE 1
Optimal anchor residues for HLA molecules

| | |
|---|---|
| HLA A*0201 | Residue 2 = L, M |
| | Residue 9 or last residue = V |
| HLA A*0301 | Residue 2 = L, M, |
| | Residue 9 or last residue = K |
| HLA A*0101 | Residue 2 = 1, S |
| | Residue 3 = D, E |
| | Residue 9 or last residue = Y |
| HLA B*2705 | Residue I = R |
| | Residue 2 = R |
| | Residue 9 or last residue L, F, K, R, M |
| HLA B*0702 | Residue 2 = P |
| | Residue 9 or last residue = L, M, V, F |
| HLA B*4402 | Residue 2 = E |
| | Residue 9 or last residue = F, Y, W |

The chemical structure of a phosphopeptide mimetic appropriate for use in the present invention may closely approximate the natural phosphorylated residue which is mimicked, and also be chemically stable (e.g., resistant to dephosphorylation by phosphatase enzymes). This can be achieved with a synthetic molecule in which the phosphorous atom is linked to the amino acid residue, not through oxygen, but through carbon. In one embodiment, a CF2 group links the amino acid to the phosphorous atom. Mimetics of several amino acids which are phosphorylated in nature can be generated by this approach. Mimetics of phosphoserine, phosphothreonine, and phosphotyrosine can be generated by placing a CF2 linkage from the appropriate carbon to the phosphate moiety. The mimetic molecule L-2-amino-4 (diethylphosphono)-4,4-difluorobutanoic acid (F2Pab) may substitute for phosphoserine (Otaka et al., Tetrahedron Letters 36: 927-930 (1995)). L-2-amino-4-phosphono-4,4difluoro-3-methylbutanoic acid (F2Pmb) may substitute for phosphothreonine. L-2-amino-4-phosphono (difluoromethyl) phenylalanine (F2Pmp) may substitute for phosphotyrosine (Akamatsu et al., Bioorg & Med Chem. 5: 157-163 (1997); Smyth et al., Tetrahedron Lett. Tetrahedron Lett. 33,4137-4140 (1992)). Alternatively, the oxygen bridge of the natural amino acid may be replaced with a methylene group.

Compositions comprising the phosphopeptide are typically substantially free of other human proteins or peptides. They can be made synthetically or by purification from a biological source. They can be made recombinantly. Desirably they are at least 90%, at least 95%, at least 99% pure. For administration to a human body, they do not contain other components that might be harmful to a human recipient. The compositions are typically devoid of cells, both human and recombinant producing cells. However, as noted below, in some cases, it may be desirable to load dendritic cells with a phosphopeptide and use those loaded dendritic cells as either an immunotherapy agent themselves, or as a reagent to stimulate a patient's T cells ex vivo. The stimulated T cells can be used as an immunotherapy agent. In some cases, it may be desirable to form a complex between a phosphopeptide and an HLA molecule of the appropriate type. Such complexes may be formed in vitro or in vivo. Such complexes are typically tetrameric with respect to an HLA-phosphopeptide complex. Under certain circumstances it may be desirable to add additional proteins or peptides, for example, to make a cocktail having the ability to stimulate an immune response in a number of different HLA type hosts. Alternatively, additional proteins or peptide can provide an interacting function within a single host, such as an adjuvant function or a stabilizing function. As an example, other tumor antigens can be used in admixture with the phosphopeptides, such that multiple different immune responses are induced in a single patient.

Administration of phosphopeptides to a mammalian recipient may be accomplished using long phosphopeptides, e.g., longer than 15 residues, or using phosphopeptide-loaded dendritic cells. See Melief, J. Med. Sciences 2009; 2:43-45. The immediate goal is to induce activation of CD8⁺ T cells. Additional components which can be administered to the same patient, either at the same time or close in time (e.g., within 21 days of each other) include TLR-ligand oligonucleotide CpG and related phosphopeptides that have overlapping sequences of at least 6 amino acid residues. To ensure efficacy, mammalian recipients should express the appropriate human HLA molecules to bind to the phosphopeptides. Transgenic mammals can be used as recipients, for example, if they express appropriate human HLA molecules. If a mammal's own immune system recognizes a similar phosphopeptide then it can be used as model system directly, without introducing a transgene. Useful models and recipients may be at increased risk of developing metastatic cancer, such as metastatic melanoma. Other useful models and recipients may be predisposed, e.g., genetically or environmentally, to develop melanoma or other cancer.

Phosphopeptide-loaded dendritic cells can also be used to transfuse a cancer patient or a patient at risk of cancer. The composition of dendritic cells can be provided with a single phosphopeptide loaded in the cells. Thus the dendritic cells are homogenous with respect to the loaded phosphopeptide. The homogeneity may not be perfectly achievable. The desired phosphopeptide may be form at least 20%, at least 50%, at least 70%, or at least 90% of the phosphopeptides loaded in the compositions. Additional components may be added to the composition to be administered, such as immune adjuvants, stabilizers, and the like. The particular phosphopeptides were identified on the surfaces of particular cancer cells, but they may be found on other types of cancer cells as well, including but not limited to melanoma, ovarian cancer, breast cancer, colorectal cancer, squamous carcinoma of the lung, sarcoma, renal cell carcinoma, pancreatic carcinomas, squamous tumors of the head and neck, leukemia, brain cancer, liver cancer, prostate cancer, ovarian cancer, and cervical cancer.

Antibodies and antibody-like molecules containing an antigen-binding region are useful, inter alia, for analyzing tissue to determine the pathological nature of tumor margins. Such tissue may be obtained from a biopsy, for example. Other samples which may be tested include blood, serum, plasma, and lymph. Antibodies to peptides may be generated using methods that are well known in the art. For the production of antibodies, various host animals, including rabbits, mice, rats, goats and other mammals, can be immunized by injection with a peptide. They may be conjugated to carrier proteins such as KLH or tetanus toxoid. Various adjuvants may be used to increase the immunological response, depending on the host species, and including but not limited to Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Guerin) and corynebacterium parvum. Methods of immunization to achieve a polyclonal antibody response are well known in the art, as are methods for generating hybridomas and monoclonal antibodies.

For preparation of monoclonal antibodies, any technique which provides for the production of antibody molecules by continuous cell lines in culture may be used. For example, the hybridoma technique originally developed by Kohler and Milstein (1975, Nature 256:495-497), as well as the trioma technique, the human B-cell hybridoma technique (Kozbor et al., 1983, Immunology Today 4:72), and the EBV-hybridoma technique to produce human monoclonal antibodies (Cole et al., 1985, in Monoclonal Antibodies and Cancer Therapy, Alan R. Liss, Inc., pp. 77-96). Monoclonal antibodies can optionally be produced in germ-free animals (see PCT/US90/02545). Human antibodies may be used and can be obtained by using human hybridomas (Cote et al., 1983, Proc. Natl. Acad. Sci. U.S.A. 80:2026-2030) or by transforming human B cells with EBV virus in vitro (Cole et al., 1985, in Monoclonal Antibodies and Cancer Therapy, Alan R. Liss, pp. 77-96). Techniques developed for the production of “chimeric antibodies” (Morrison et al., 1984, Proc. Natl. Acad. Sci. U.S.A. 81:6851-6855; Neuberger et al., 1984, Nature 312:604-608; Takeda et al., 1985, Nature 314:452-454) by splicing the genes from a mouse antibody molecule specific for desired epitopes together with genes from a human antibody molecule of appropriate biological activity can be used.

Antibodies may include, but are not limited to, polyclonal, monoclonal, chimeric (i.e., “humanized” antibodies), single chain (recombinant), Fab fragments, and fragments produced by a Fab expression library. Any of these molecules which contain an antigen binding region specific for a phosphopeptide relative to its cognate non-phosphorylated peptide may be used. These molecules can be used as diagnostic agents for the diagnosis of conditions or diseases (such as cancer) characterized by expression or overexpression of antigen peptides, or in assays to monitor a patient's responsiveness to an anti-cancer therapy. Antibodies specific for one or more of the antigen phosphopeptides can be used as diagnostics for the detection of the antigen phosphopeptides in cancer cells.

The antibodies or antibody fragments of the present invention can be combined with a carrier or diluent to form a composition. In one embodiment, the carrier is a pharmaceutically acceptable carrier. Such carriers and diluents include sterile liquids such as water and oils, with or without the addition of a surfactant and other pharmaceutically and physiologically acceptable carrier, including adjuvants, excipients or stabilizers. Illustrative oils are those of petroleum, animal, vegetable, or synthetic origin, for example, peanut oil, soybean oil, or mineral oil. In general, water, saline, aqueous dextrose, and related sugar solution, and glycols such as, propylene glycol or polyethylene glycol, are preferred liquid carriers, particularly for injectable solutions.

The antigen phosphopeptides are known to be expressed on a variety of cancer cell types. Thus, they can be used where appropriate, in treating, diagnosing, vaccinating, preventing, retarding, and attenuating melanoma, ovarian cancer, breast cancer, colorectal cancer, squamous carcinoma of the lung, sarcoma, renal cell carcinoma, pancreatic carcinomas, squamous tumors of the head and neck, leukemia, brain cancer, liver cancer, prostate cancer, ovarian cancer, and cervical cancer.

Antibodies generated with specificity for the antigen phosphopeptides can be used to detect the corresponding phosphopeptides in biological samples. The biological sample could come from an individual who is suspected of having cancer and thus detection would serve to diagnose the cancer. Alternatively, the biological sample may come from an individual known to have cancer, and detection of the antigen phosphopeptides would serve as an indicator of disease prognosis, cancer characterization, or treatment efficacy. Appropriate immunoassays are well known in the art and include, but are not limited to, immunohistochemistry, flow cytometry, radioimmunoassay, western blotting, and ELISA. Biological samples suitable for such testing include, but are not limited to, cells, tissue biopsy specimens, whole blood, plasma, serum, sputum, cerebrospinal fluid, pleural fluid, and urine. Antigens recognized by T cells, whether helper T lymphocytes or CTL, are not recognized as intact proteins, but rather as small peptides that associate with class I or class II MHC proteins on the surface of cells. During the course of a naturally occurring immune response antigens that are recognized in association with class II MHC molecules on antigen presenting cells are acquired from outside the cell, internalized, and processed into small peptides that associate with the class II MHC molecules. Conversely, the antigens that give rise to proteins that are recognized in association with class I MHC molecules are generally proteins made within the cells, and these antigens are processed and associate with class I MHC molecules. It is now well known that the peptides that associate with a given class I or class II MHC molecule are characterized as having a common binding motif, and the binding motifs for a large number of different class I and II MHC molecules have been determined. It is also well known that synthetic peptides can be made which correspond to the sequence of a given antigen and which contain the binding motif for a given class I or II MHC molecule. These peptides can then be added to appropriate antigen presenting cells, and the antigen presenting cells can be used to stimulate a T helper cell or CTL response either in vitro or in vivo. The binding motifs, methods for synthesizing the peptides, and methods for stimulating a T helper cell or CTL response are all well known and readily available.

Kits may be composed for help in diagnosis, monitoring, or prognosis. The kits are to facilitate the detecting and/or measuring cancer-specific phosphoproteins. Such kits contain in a single or divided container, a molecule comprising an antigen-binding region. Such molecules are antibodies or antibody-like molecules. Additional components which may be included in the kit include solid supports, detection reagents, secondary antibodies, instructions for practicing, vessels for running assays, gels, control samples, and the like. The antibody or antibody-like molecules may be directly labeled, as an option.

The antigens of this invention may take the form of antigen peptides added to autologous dendritic cells and used to stimulate a T helper cell or CTL response in vitro. The in vitro generated T helper cells or CTL can then be infused into a patient with cancer (Yee et al., 2002), and specifically a patient with a form of cancer that expresses one or more of antigen phosphopeptides. The antigen phosphopeptides may also be used to vaccinate an individual. The antigen phosphopeptides may be injected alone, but most often they would be administered in combination with an adjuvant. The phosphopeptides may also be added to dendritic cells in vitro, with the loaded dendritic cells being subsequently transferred into an individual with cancer in order to stimulate an immune response. Alternatively, the loaded dendritic cells may be used to stimulate CD8⁺ T cells ex vivo with subsequent reintroduction of the stimulated T cells to the patient. Although a particular phosphopeptide may be identified on a particular cancer cell type, it may be found on other cancer cell types. Thus a particular phosphopeptide may have use for treating and vaccinating against multiple cancer types.

Phosphopeptide analogs can readily be synthesized that retain their ability to stimulate a particular immune response, but which also gain one or more beneficial features, such as those described below.
a. Substitutions may be made in the phosphopeptide at residues known to interact with the MHC molecule. Such substitutions can have the effect of increasing the binding affinity of the phosphopeptide for the MHC molecule and can also increase the half-life of the phosphopeptide-MHC complex, the consequence of which is that the analog is a more potent stimulator of an immune response than is the original peptide.
b. Additionally, the substitutions may have no effect on the immunogenicity of the phosphopeptide per se, but rather than may prolong its biological half-life or prevent it from undergoing spontaneous alterations which might otherwise negatively impact on the immunogenicity of the peptide.

The antigen phosphopeptides of this invention can also be used as a vaccine for cancer, and more specifically for melanoma, leukemia, ovarian, breast, colorectal, or lung squamous cancer, sarcoma, renal cell carcinoma, pancreatic carcinomas, squamous tumors of the head and neck, brain cancer, liver cancer, prostate cancer, ovarian cancer, and cervical cancer. The antigens may take the form of phosphoproteins, or phosphopeptides. The vaccine may include only the antigens of this invention or they may include other cancer antigens that have been identified. Pharmaceutical carriers, diluents and excipients are generally added that are compatible with the active ingredients and acceptable for pharmaceutical use. Examples of such carriers include, but are not limited to, water, saline solutions, dextrose, or glycerol. Combinations of carriers may also be used. The vaccine compositions may further incorporate additional substances to stabilize pH, or to function as adjuvants, wetting agents, or emulsifying agents, which can serve to improve the effectiveness of the vaccine.

The composition may be administered parenterally, either systemically or topically. Parenteral routes include subcutaneous, intravenous, intradermal, intramuscular, intraperitoneal, intranasal, transdermal, or buccal routes. One or more such routes may be employed. Parenteral administration can be, for example, by bolus injection or by gradual perfusion over time. Alternatively, or concurrently, administration may be by the oral route.

It is understood that a suitable dosage of an immunogen will depend upon the age, sex, health, and weight of the recipient, the kind of concurrent treatment, if any, the frequency of treatment, and the nature of the effect desired, however, the most preferred dosage can be tailored to the individual subject, as determined by the researcher or clinician. The total dose required for any given treatment will commonly be determined with respect to a standard reference dose based on the experience of the researcher or clinician, such dose being administered either in a single treatment or in a series of doses, the success of which will depend on the production of a desired immunological result (i.e., successful production of a T helper cell and/or CTL-mediated response to the antigen, which response gives rise to the prevention and/or treatment desired). Thus, the overall administration schedule must be considered in determining the success of a course of treatment and not whether a single dose, given in isolation, would or would not produce the desired immunologically therapeutic result or effect. Thus, the therapeutically effective amount (i.e., that producing the desired T helper cell and/or CTL-mediated response) will depend on the antigenic composition of the vaccine used, the nature of the disease condition, the severity of the disease condition, the extent of any need to prevent such a condition where it has not already been detected, the manner of administration dictated by the situation requiring such administration, the weight and state of health of the individual receiving such administration, and the sound judgment of the clinician or researcher. Needless to say, the efficacy of administering additional doses, and of increasing or decreasing the interval, may be re-evaluated on a continuing basis, in view of the recipient's immunocompetence (for example, the level of T helper cell and/or CTL activity with respect to tumor-associated or tumor-specific antigens).

The concentration of the T helper or CTL stimulatory peptides of the invention in pharmaceutical formulations are subject to wide variation, including anywhere from less than 0.01% by weight to as much as 50% or more. Factors such as volume and viscosity of the resulting composition should also be considered. The solvents, or diluents, used for such compositions include water, possibly PBS (phosphate buffered saline), or saline itself, or other possible carriers or excipients. The immunogens of the present invention may also be contained in artificially created structures such as liposomes, which structures may or may not contain additional molecules, such as proteins or polysaccharides, inserted in the outer membranes of said structures and having the effect of targeting the liposomes to particular areas of the body, or to particular cells within a given organ or tissue. Such targeting molecules may commonly be some type of immunoglobulin. Antibodies may work particularly well for targeting the liposomes to tumor cells.

The vaccine compositions may be used prophylactically for the purposes of preventing, reducing the risk of, delaying initiation of a cancer in an individual that does not currently have cancer. Or they may be used to treat an individual that already has cancer, so that recurrence or metastasis is delayed or prevented. Prevention relates to a process of prophylaxis in which the individual is immunized prior to the induction or onset of cancer. For example, individuals with a history of severe sunburn and at risk for developing melanoma, might be immunized prior to the onset of the disease. Alternatively, individuals that already have cancer can be immunized with the antigens of the present invention so as to stimulate an immune response that would be reactive against the cancer. A clinically relevant immune response would be one in which the cancer partially or completely regresses and is eliminated from the patient, and it would also include those responses in which the progression of the cancer is blocked without being eliminated. Similarly, prevention need not be total, but may result in a reduced risk, delayed onset, or delayed progression or metastasis.

The above disclosure generally describes the present invention. All references disclosed herein are expressly incorporated by reference. A more complete understanding can be obtained by reference to the following specific examples which are provided herein for purposes of illustration only, and are not intended to limit the scope of the invention.

### EXAMPLE 1

The present example encompasses inter alia a set of phosphorylated peptides presented by HLA A*0101, A*0301 and B*4402 on the surface of melanoma cells that have the potential to (a) stimulate an immune response to the cancer, (b) to function as immunotherapeutics in adoptive T-cell therapy or as a vaccine, (c) to facilitate antibody recognition of the tumor boundaries in surgical pathology samples, and (d) act as biomarkers for early detection of the disease. The present invention provides at least 246 class I MHC peptides presented on the surface of melanoma cells in association with the HLA molecules A*0101, A*0301, and B*4402.

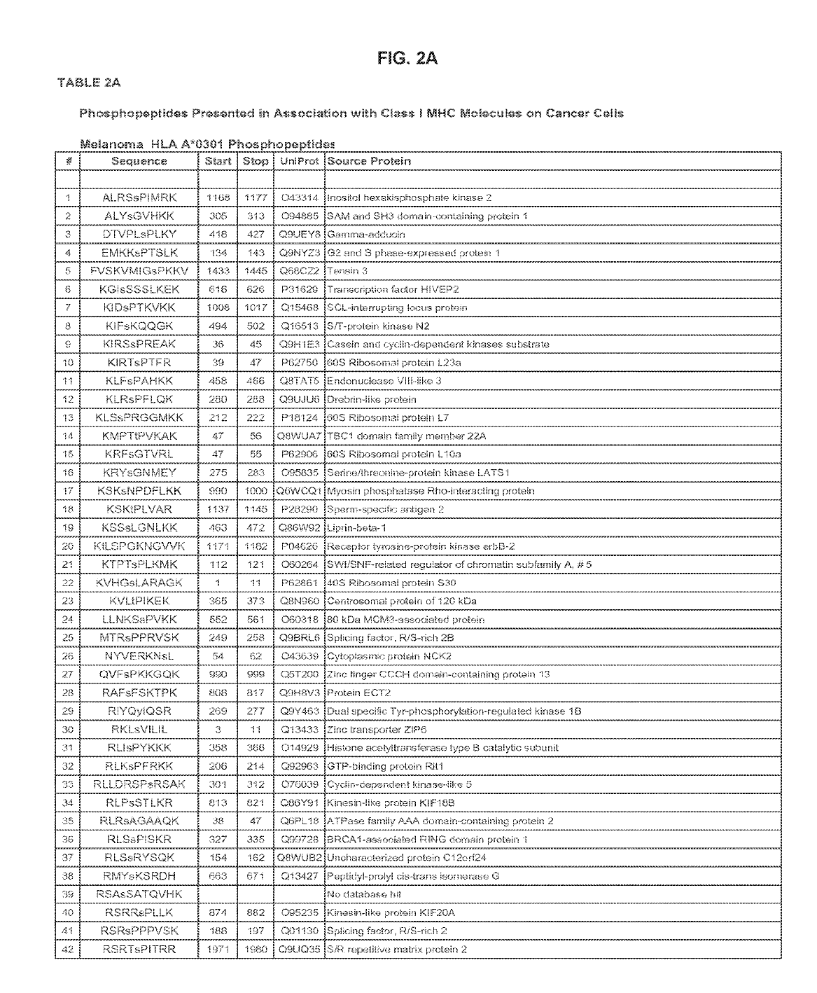

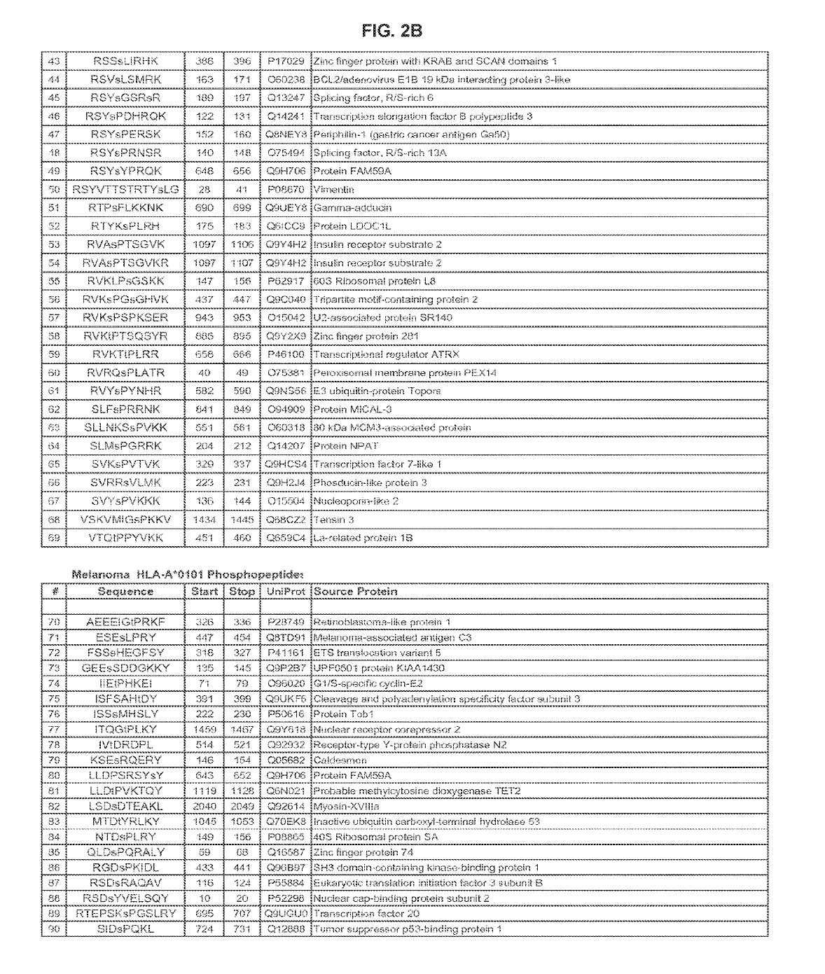

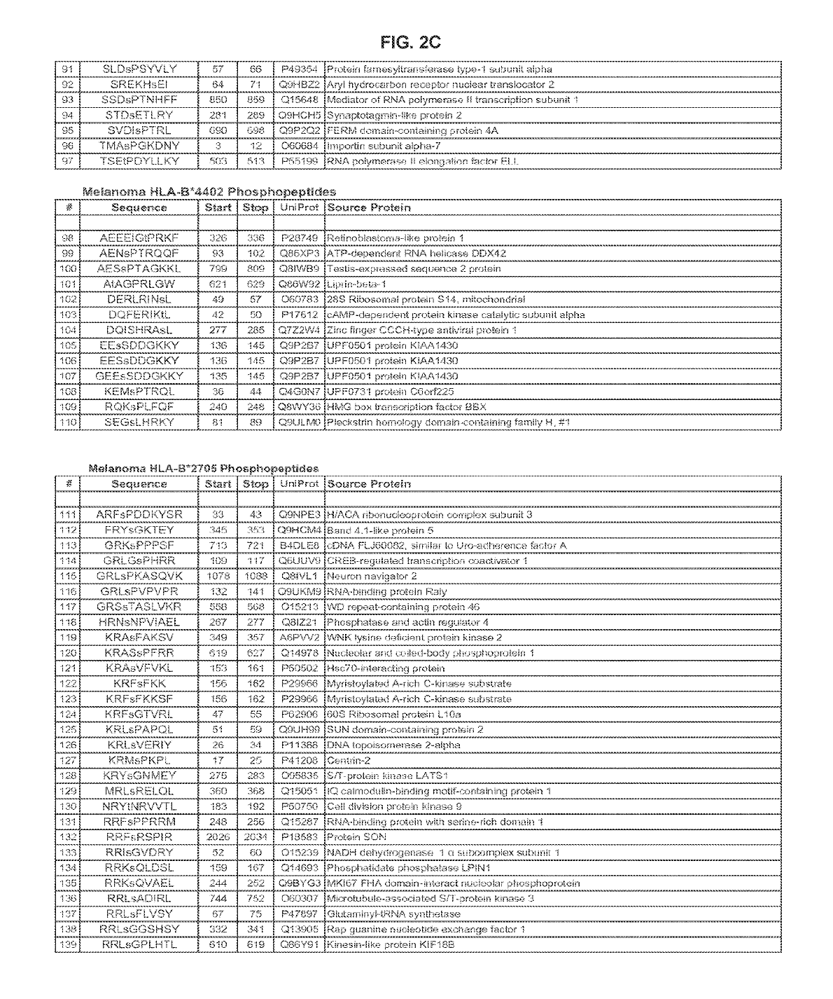

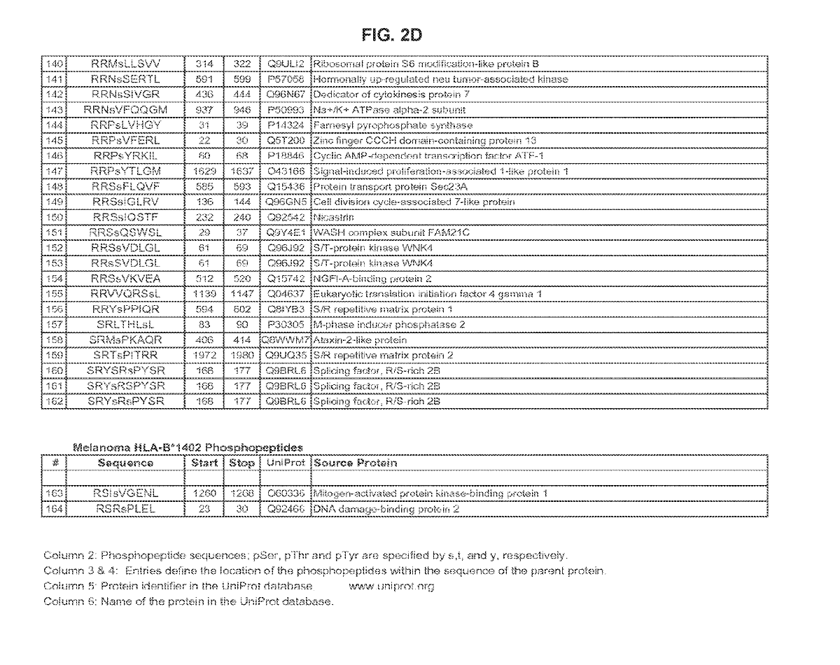

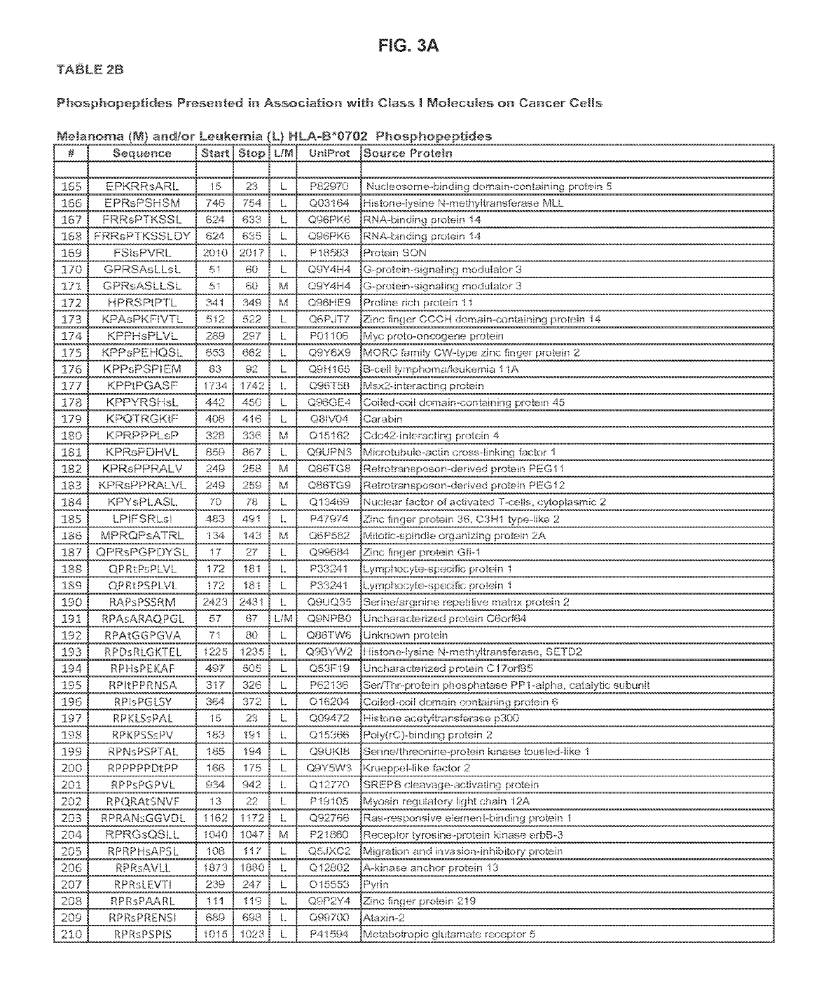

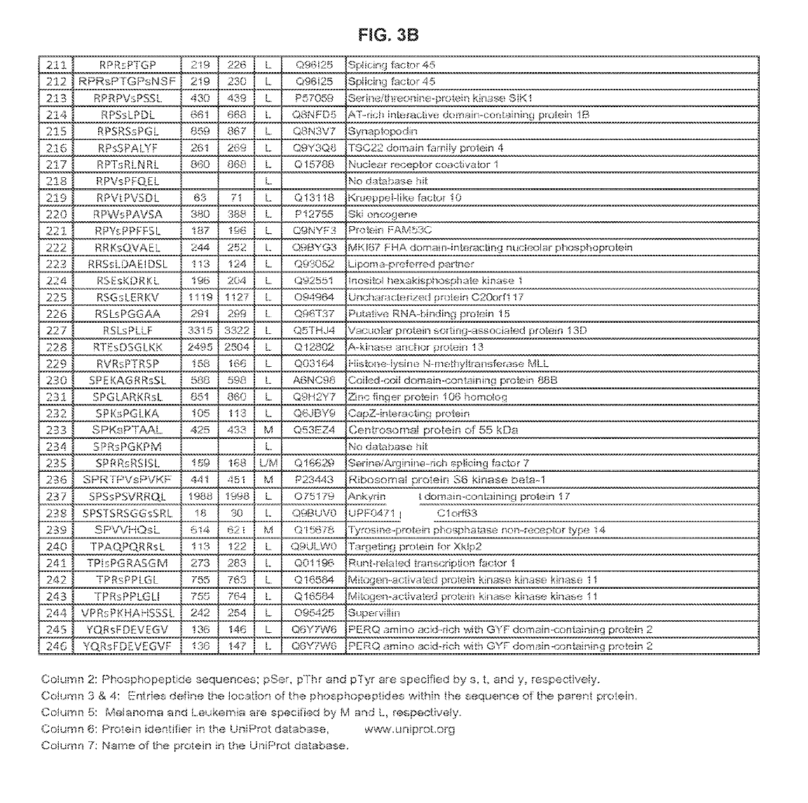

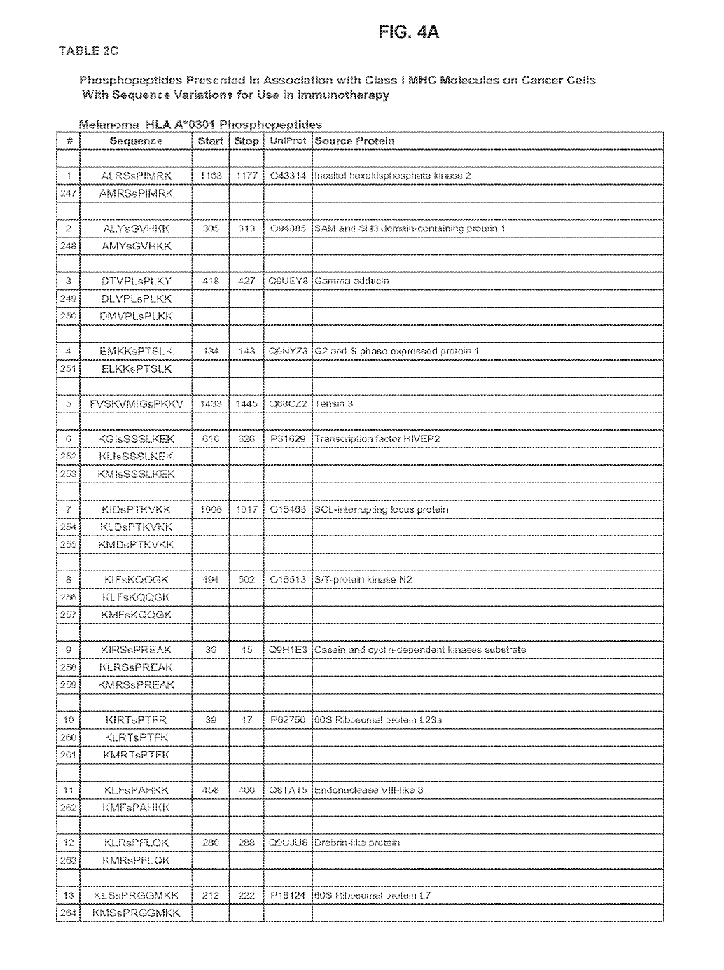

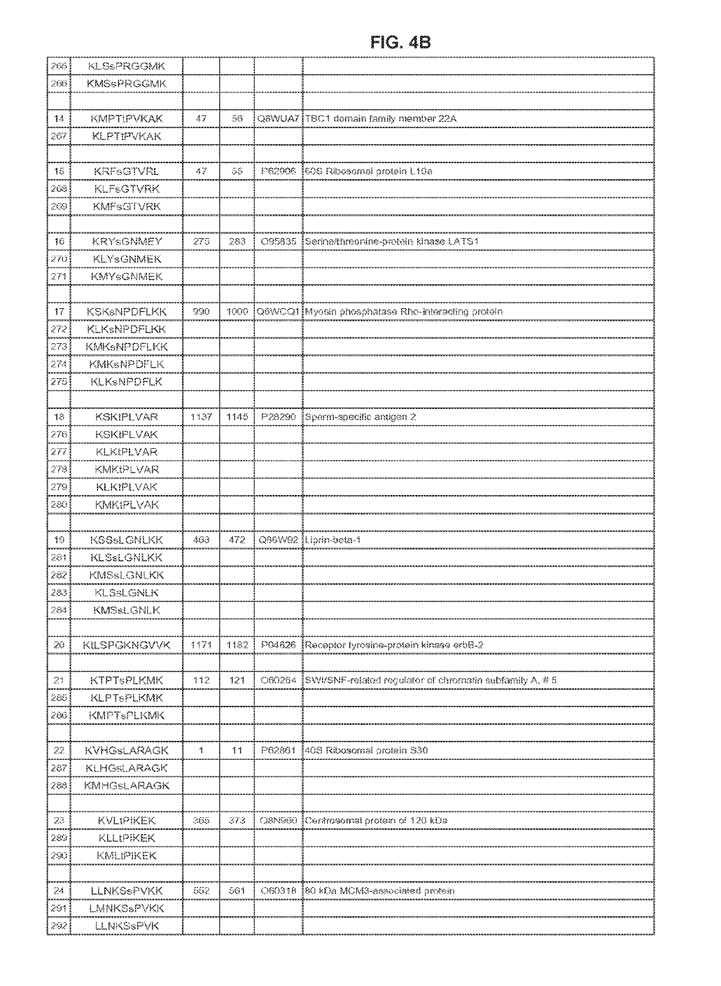

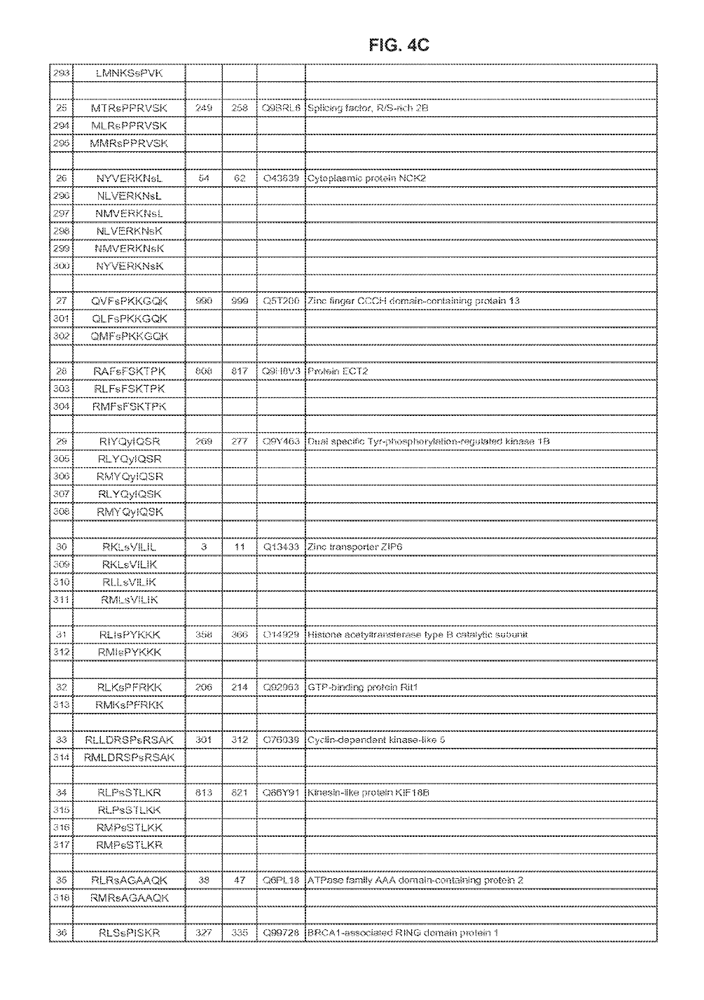

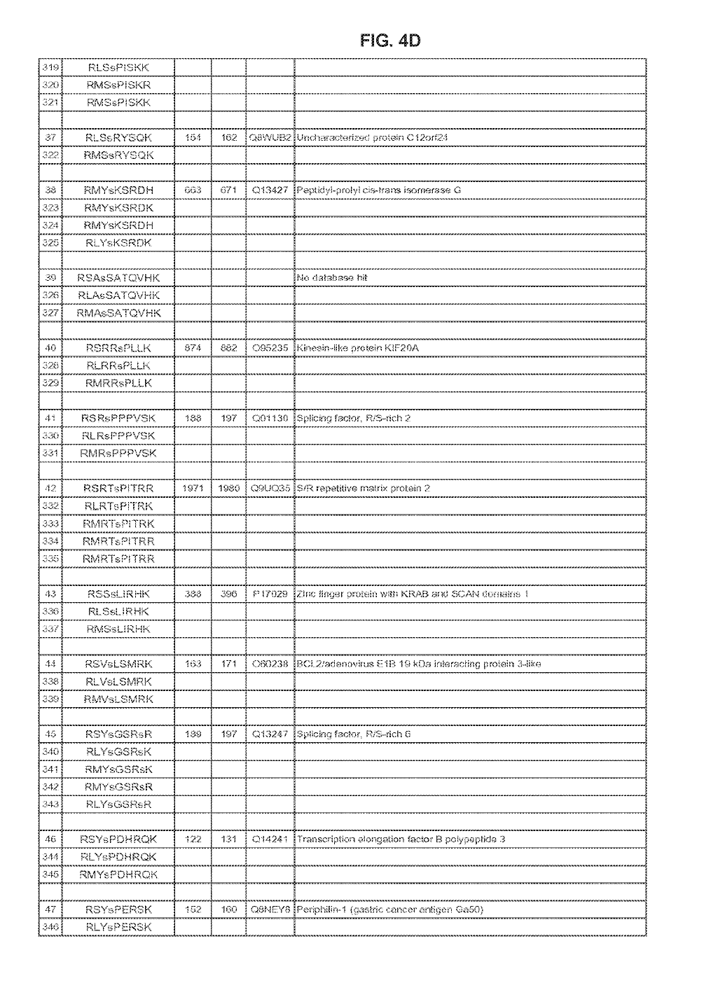

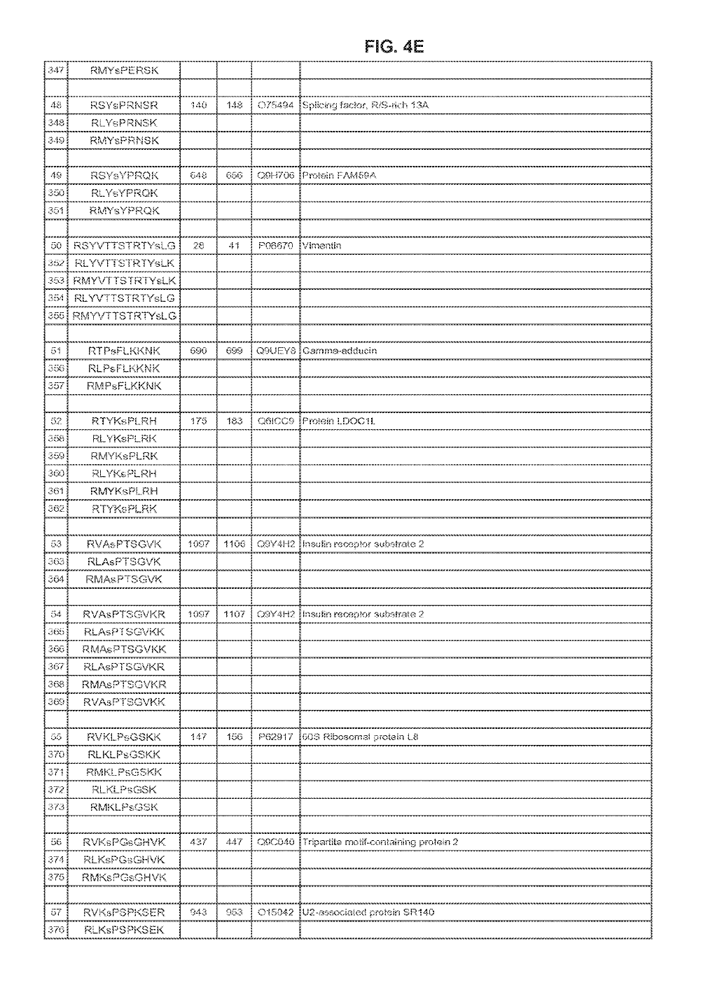

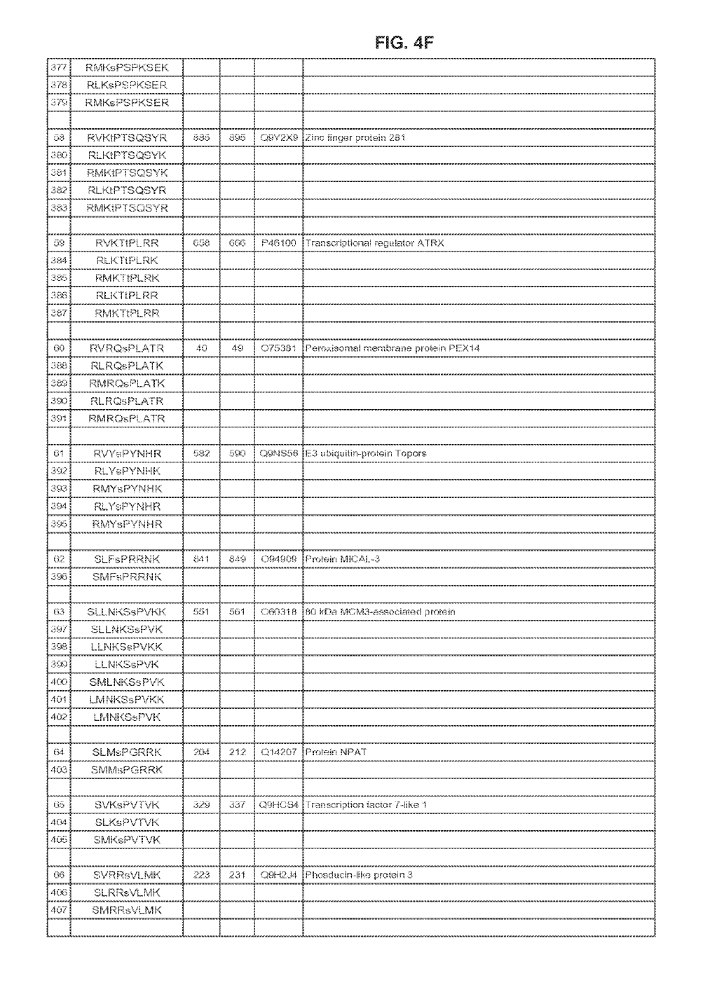

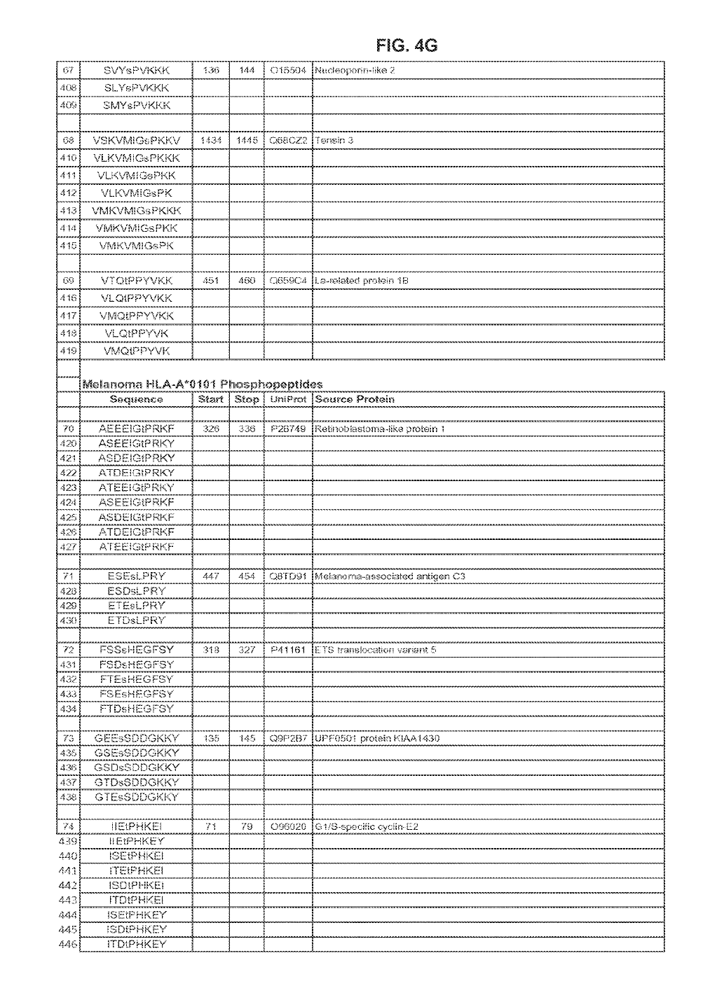

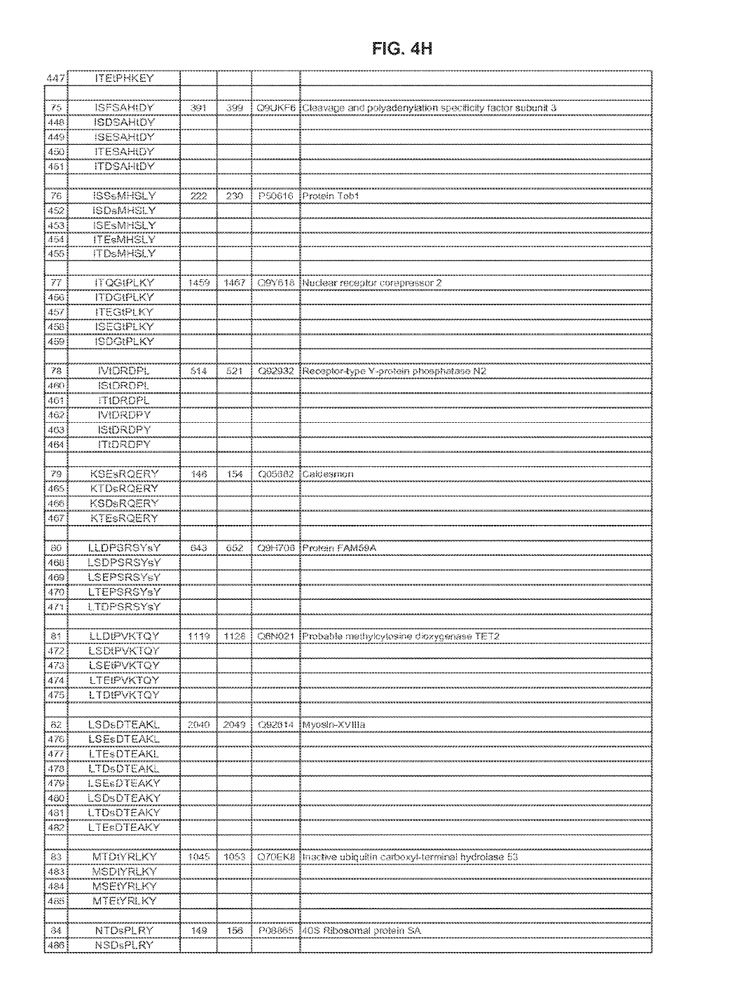

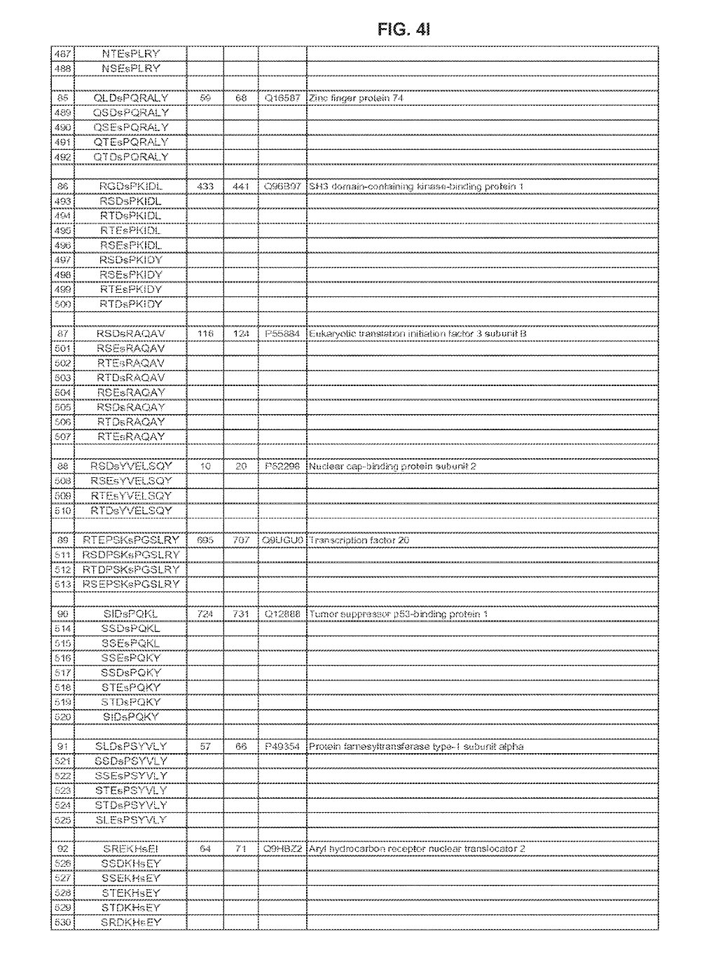

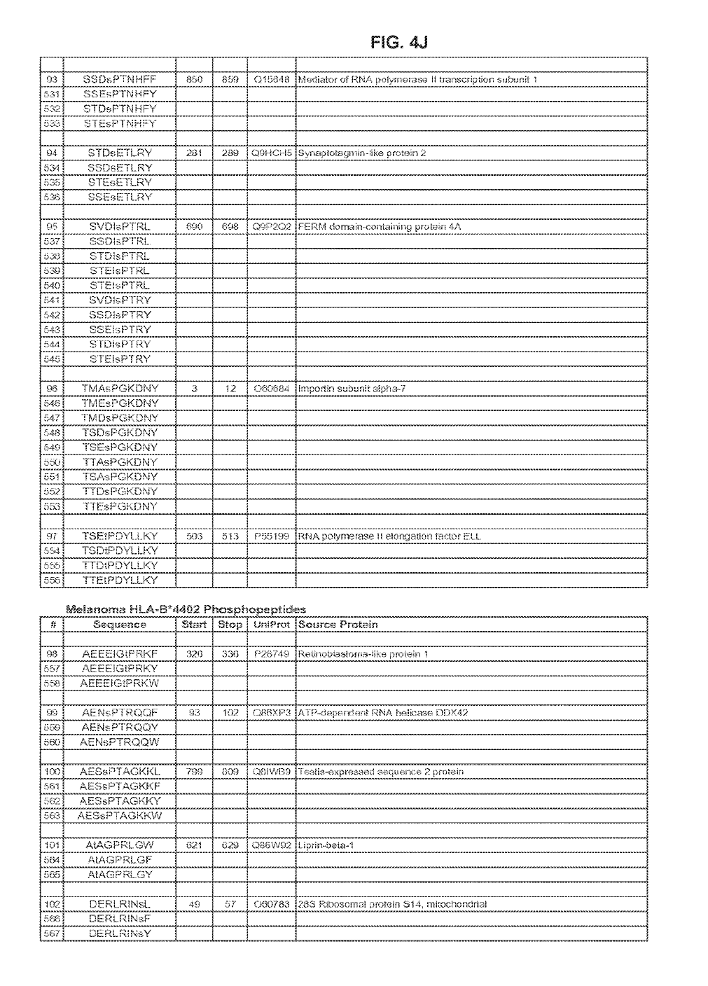

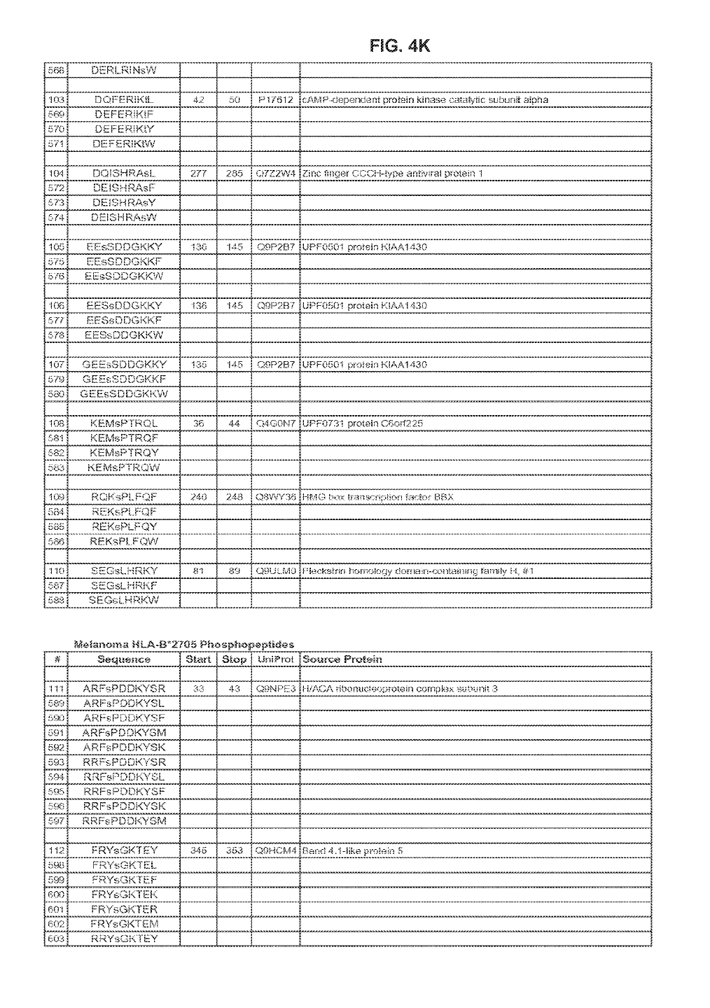

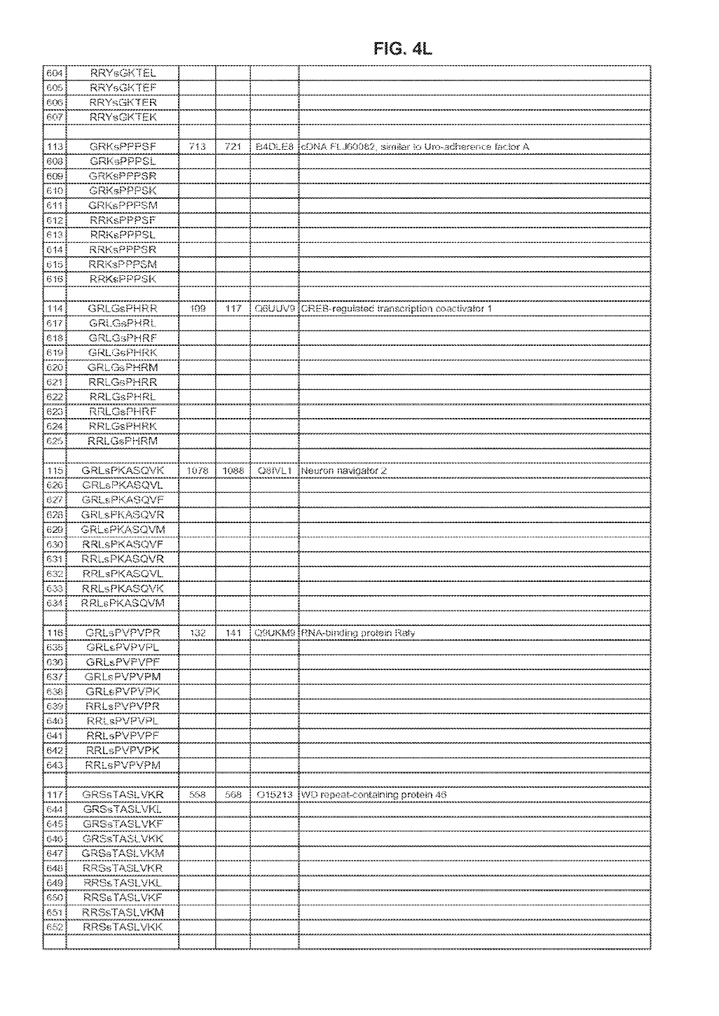

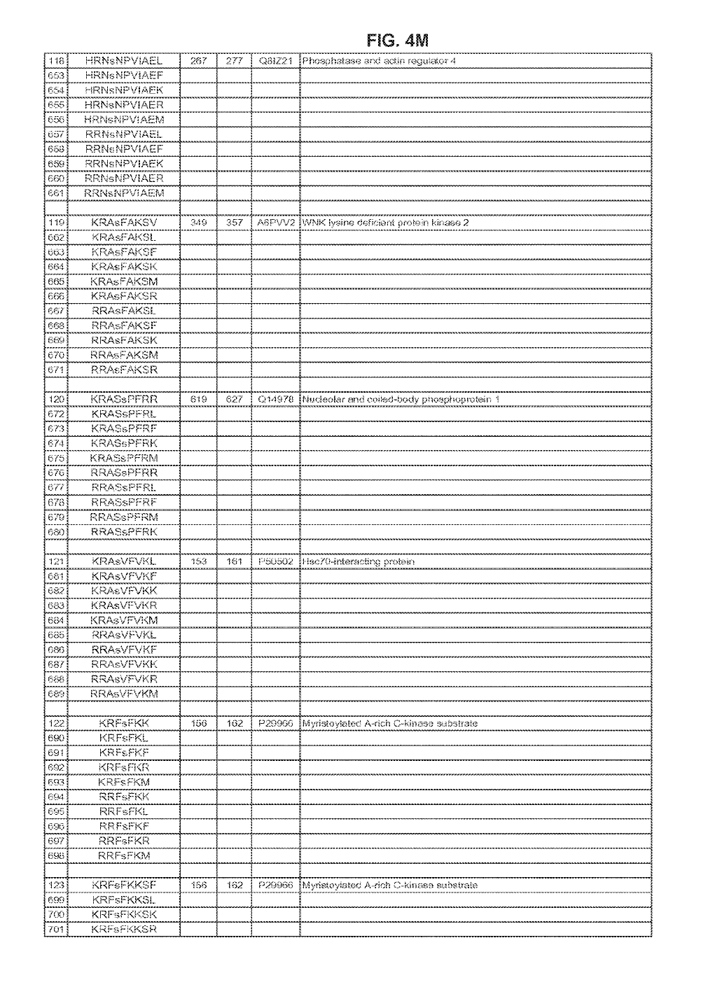

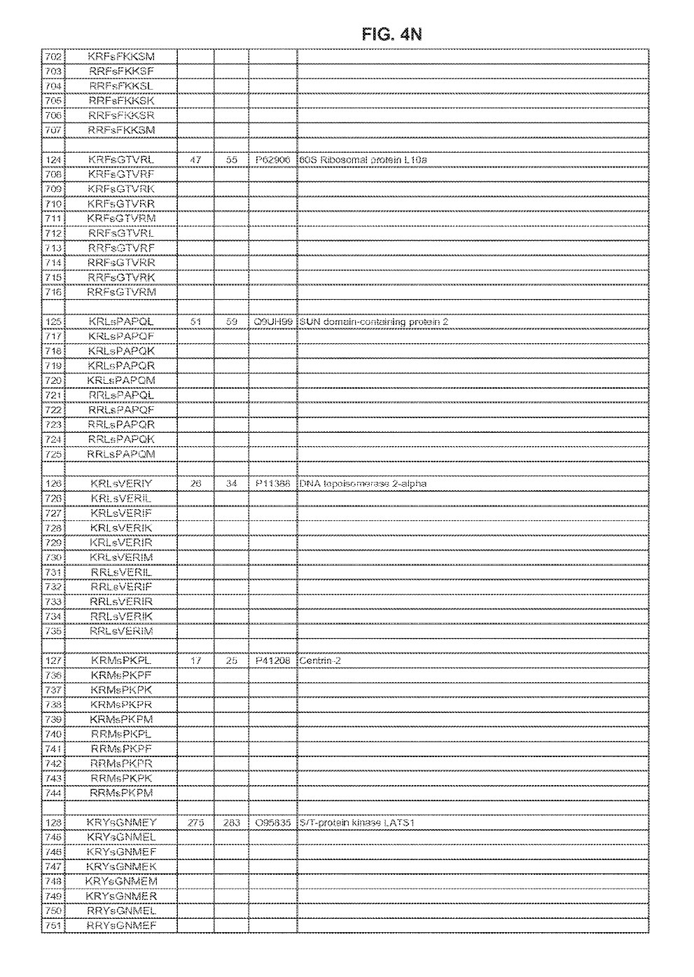

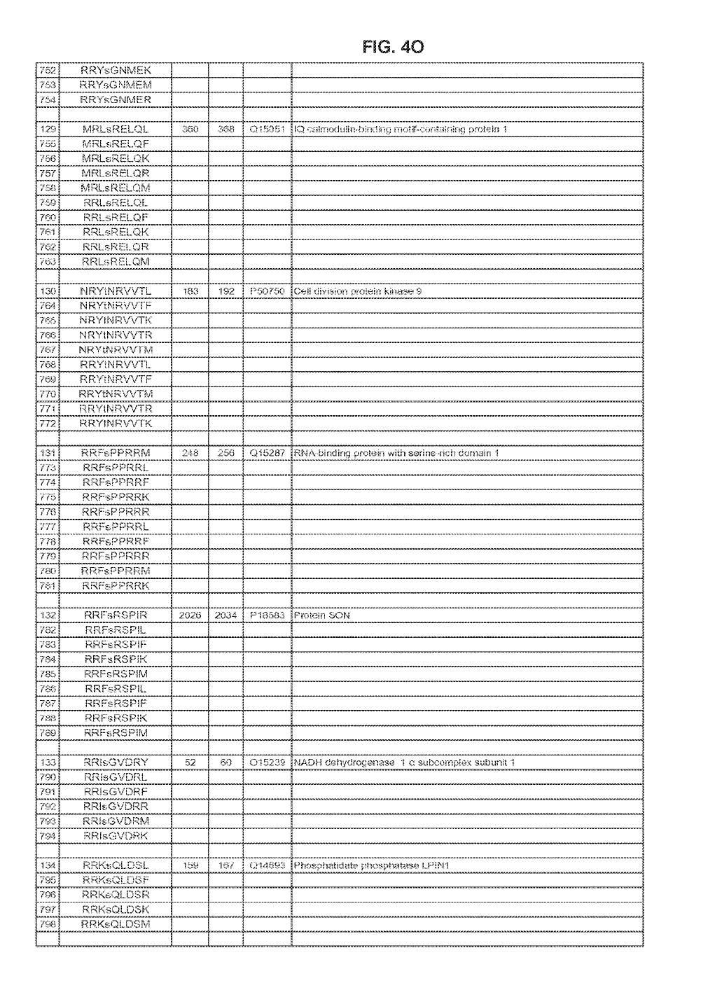

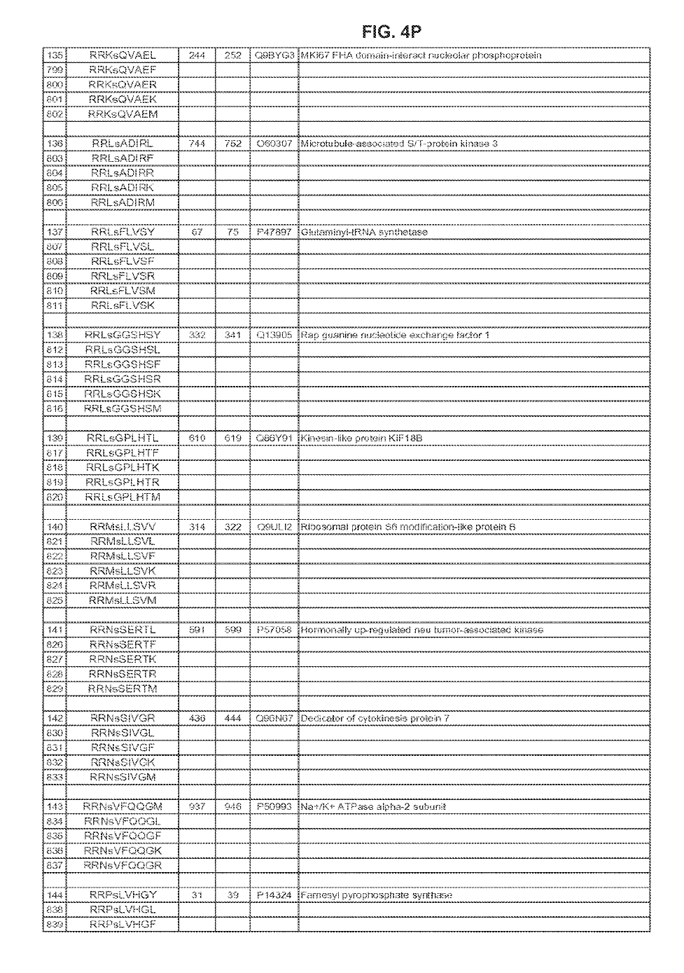

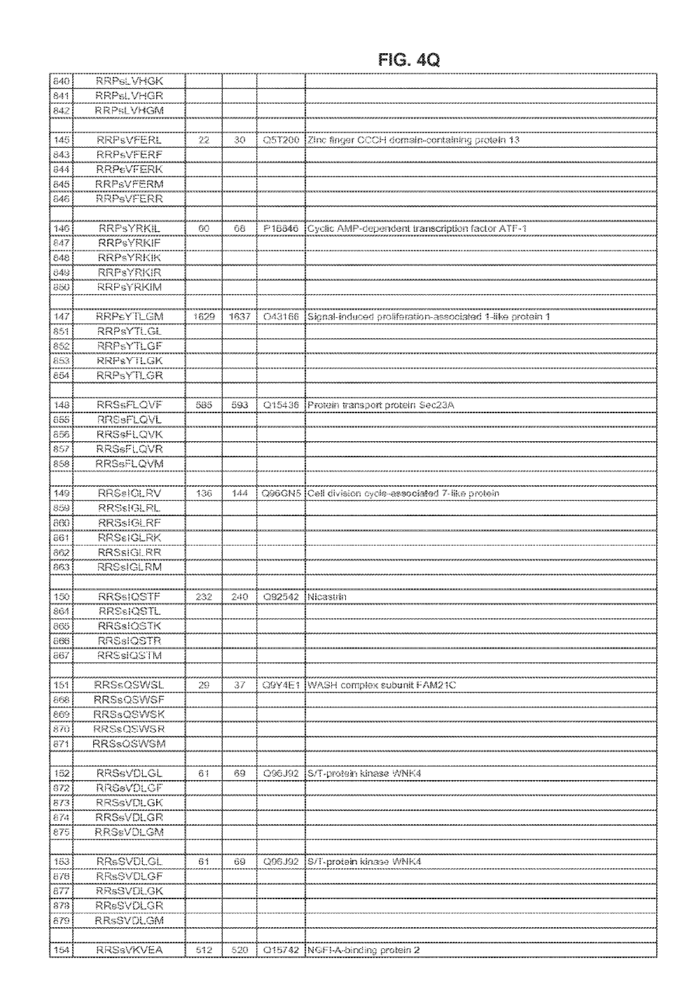

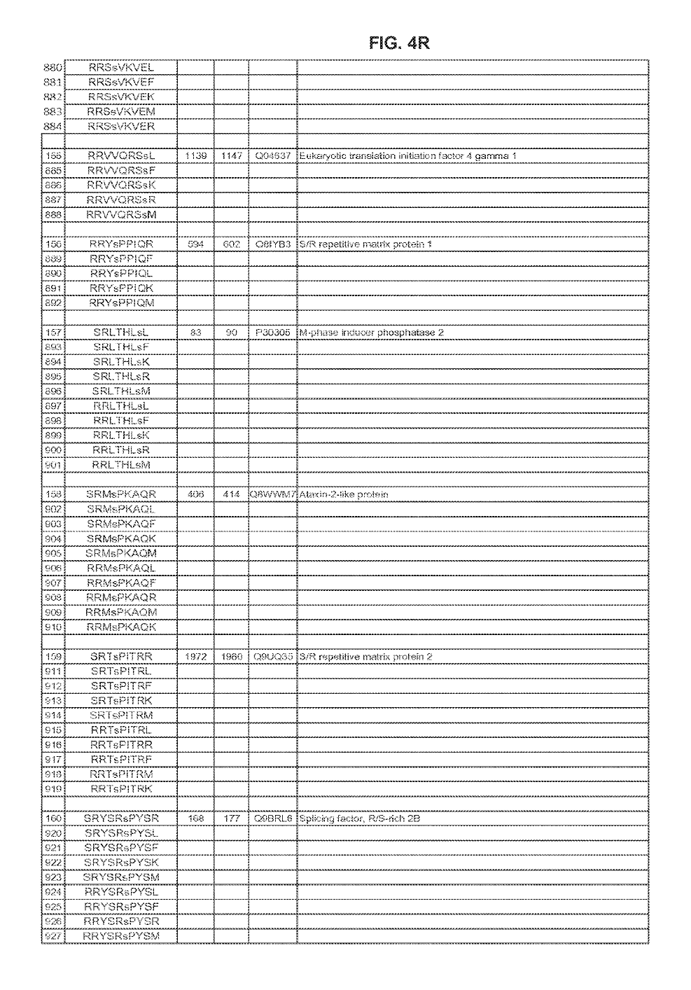

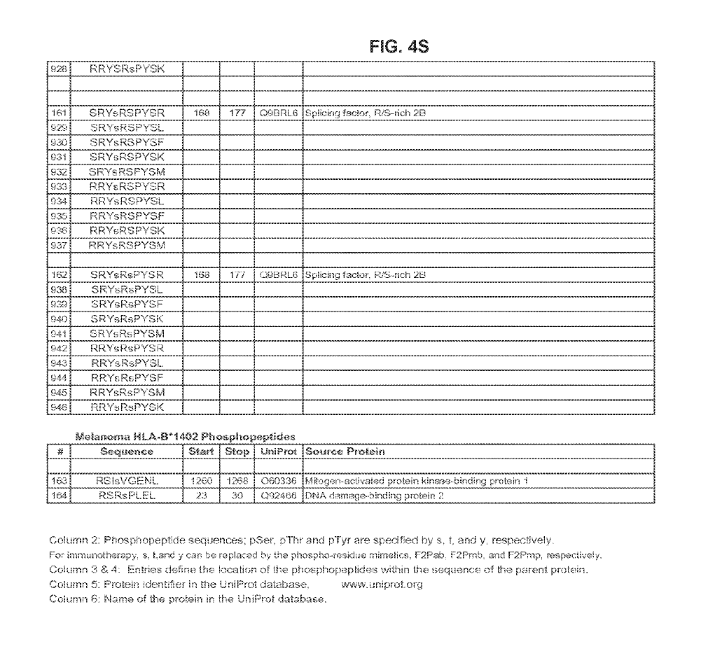

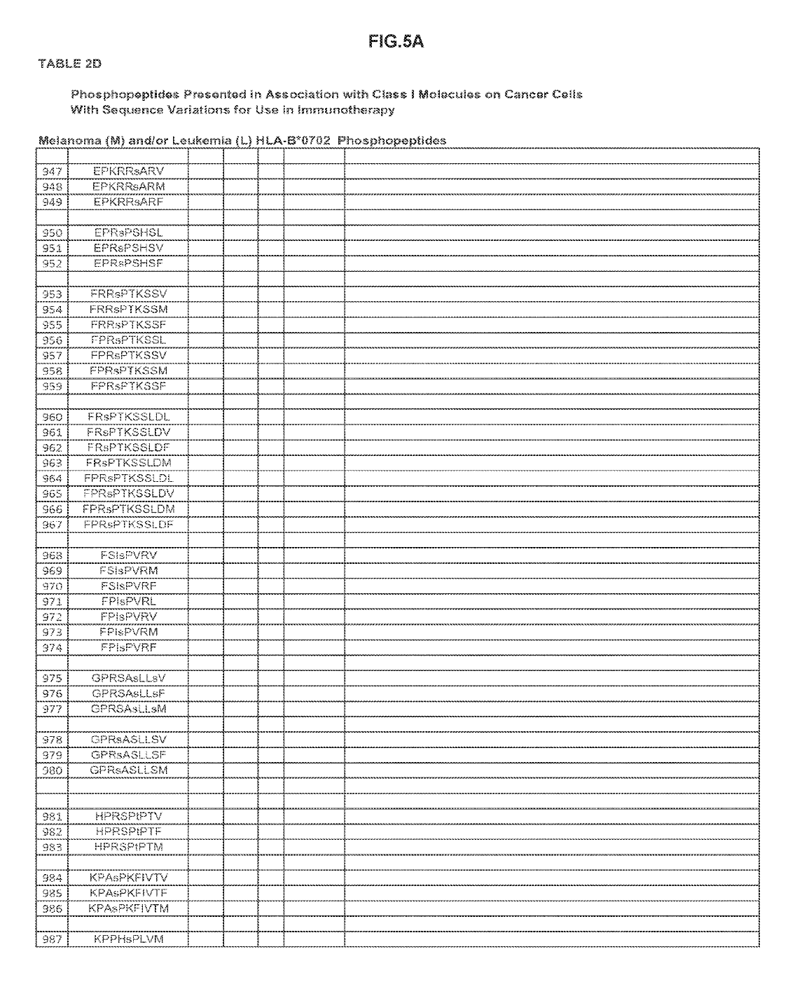

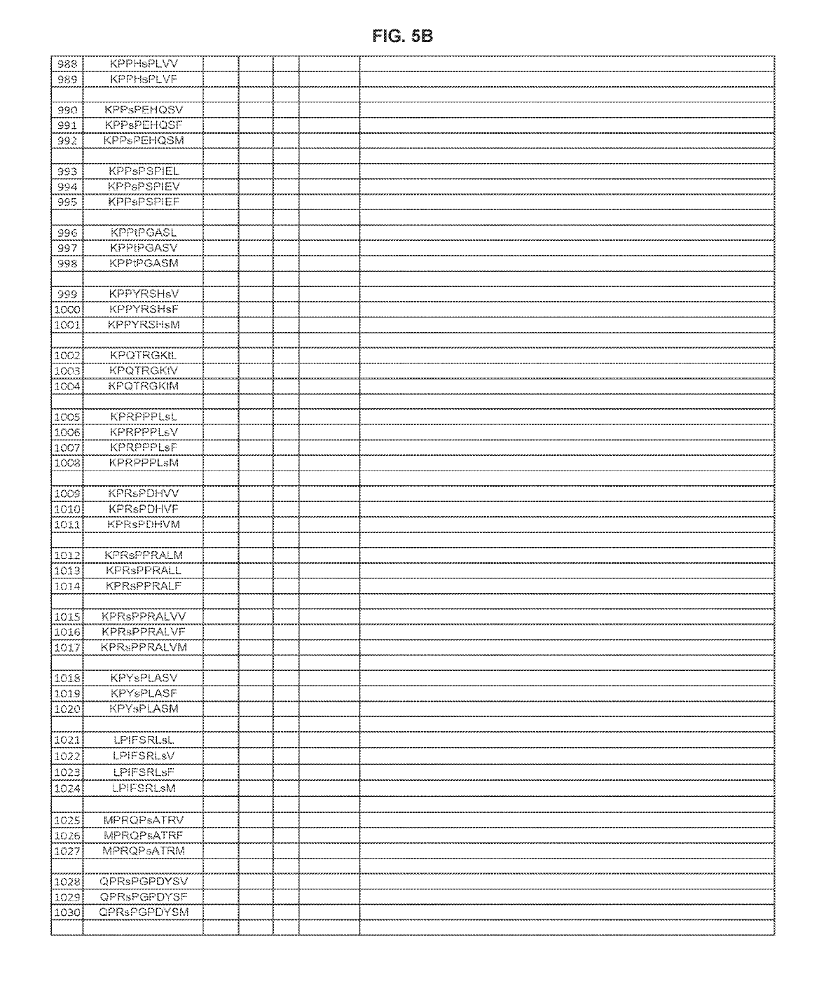

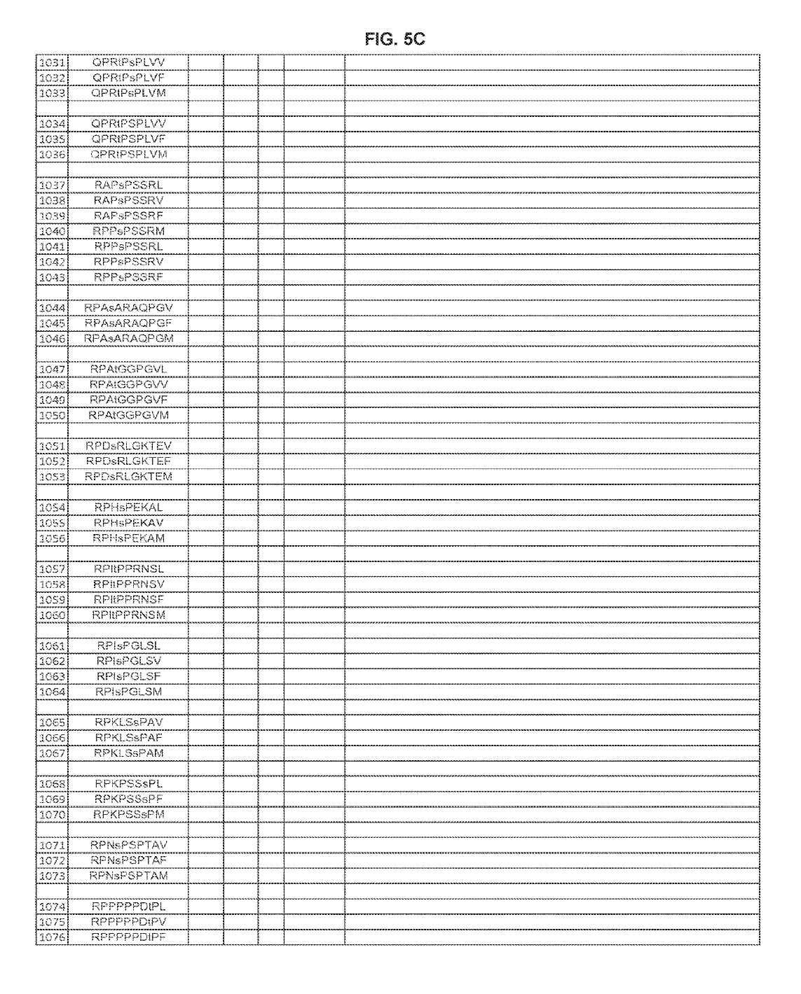

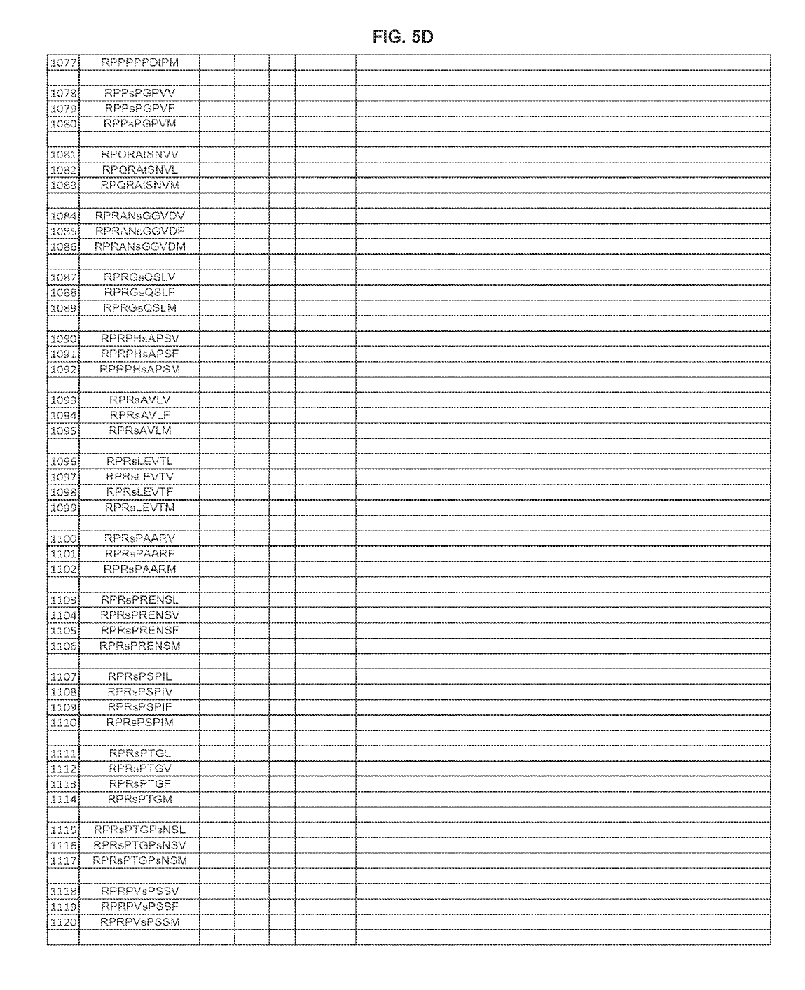

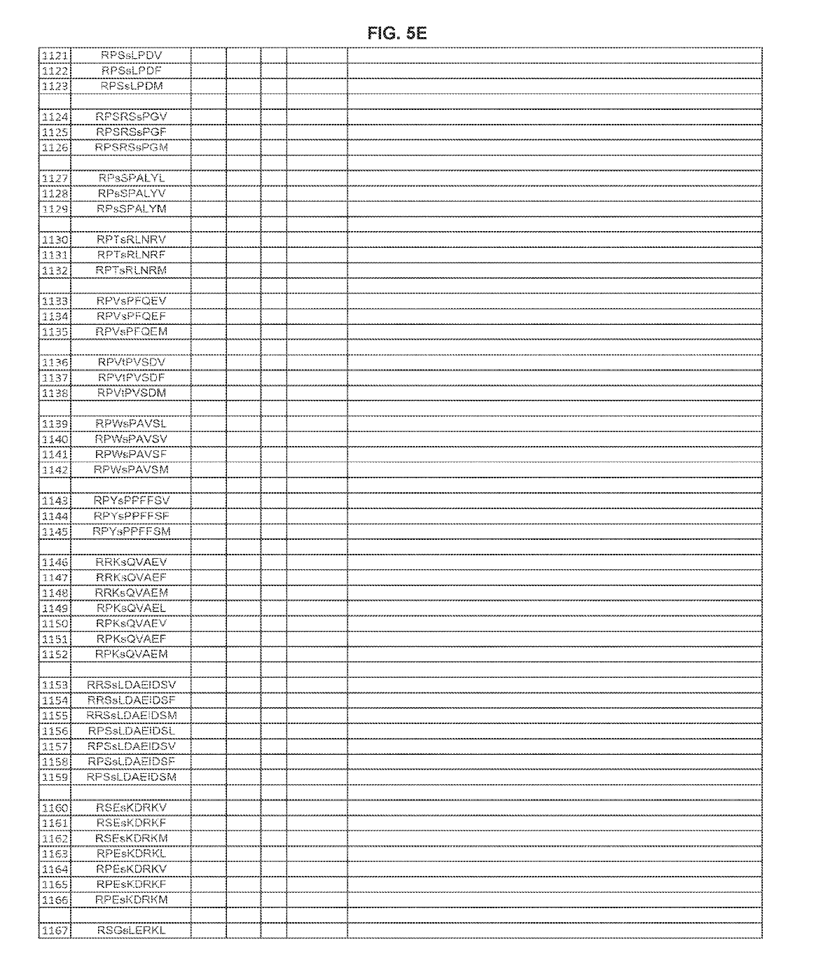

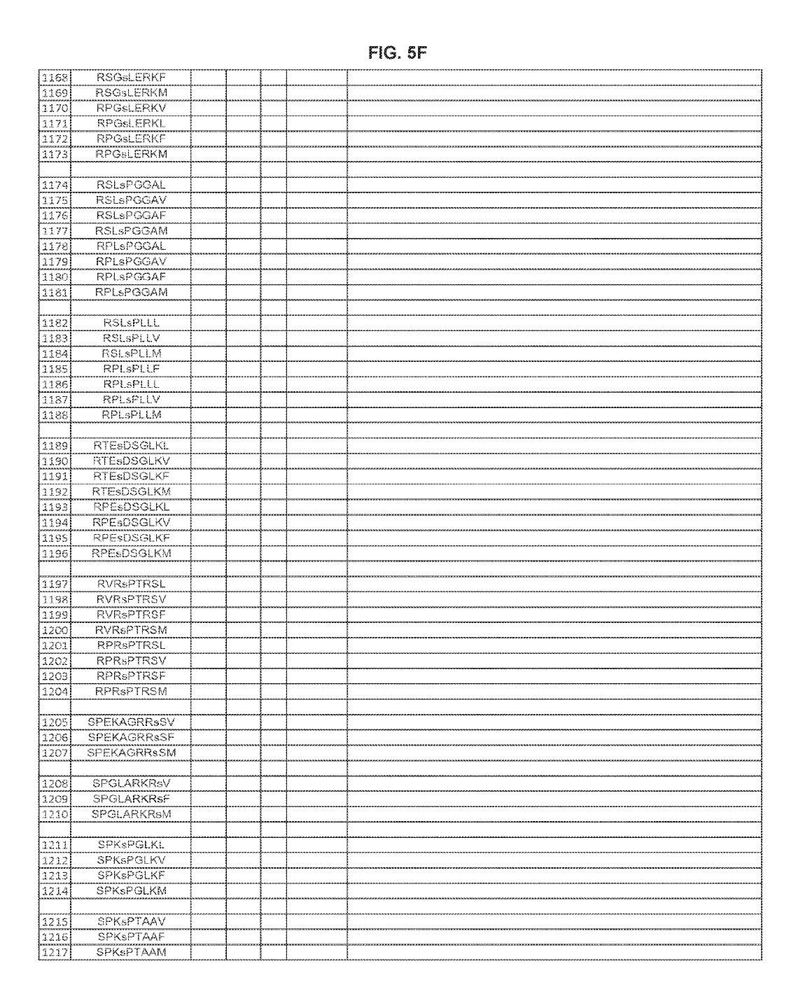

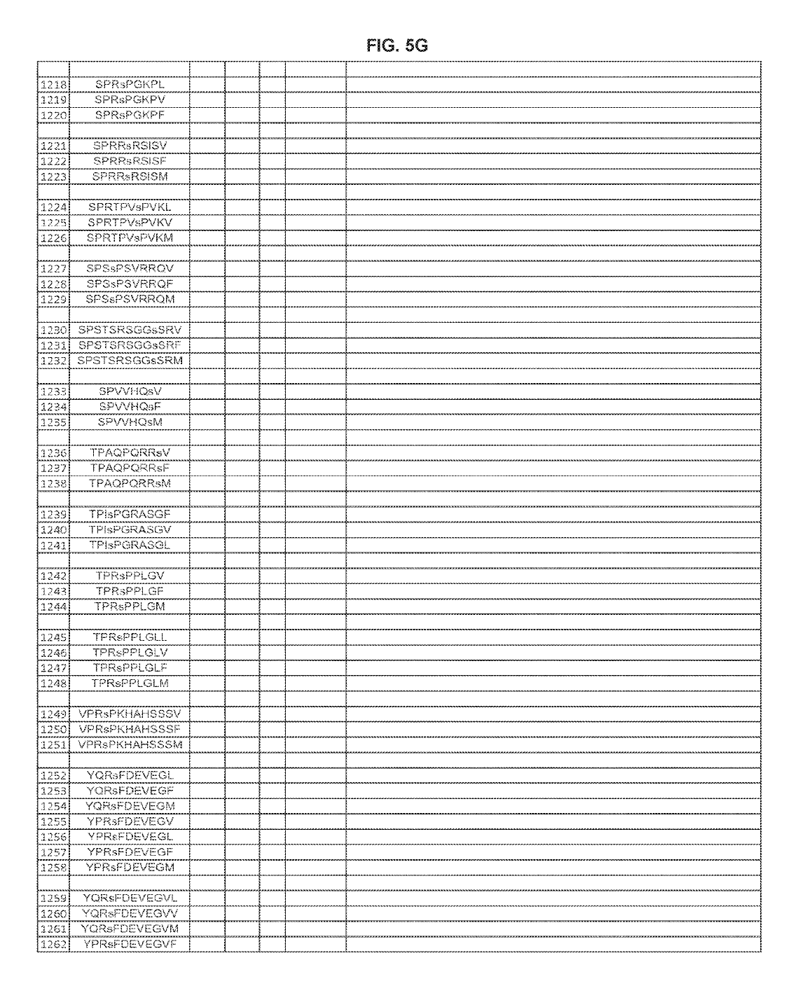

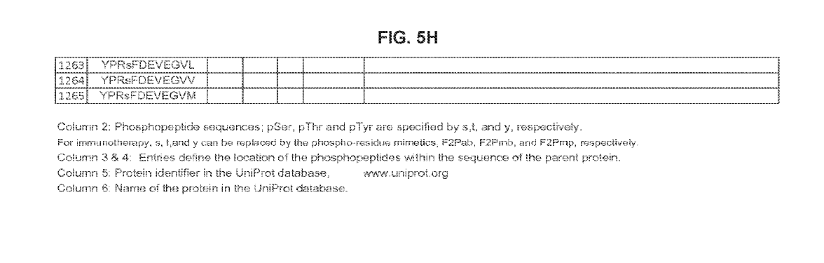

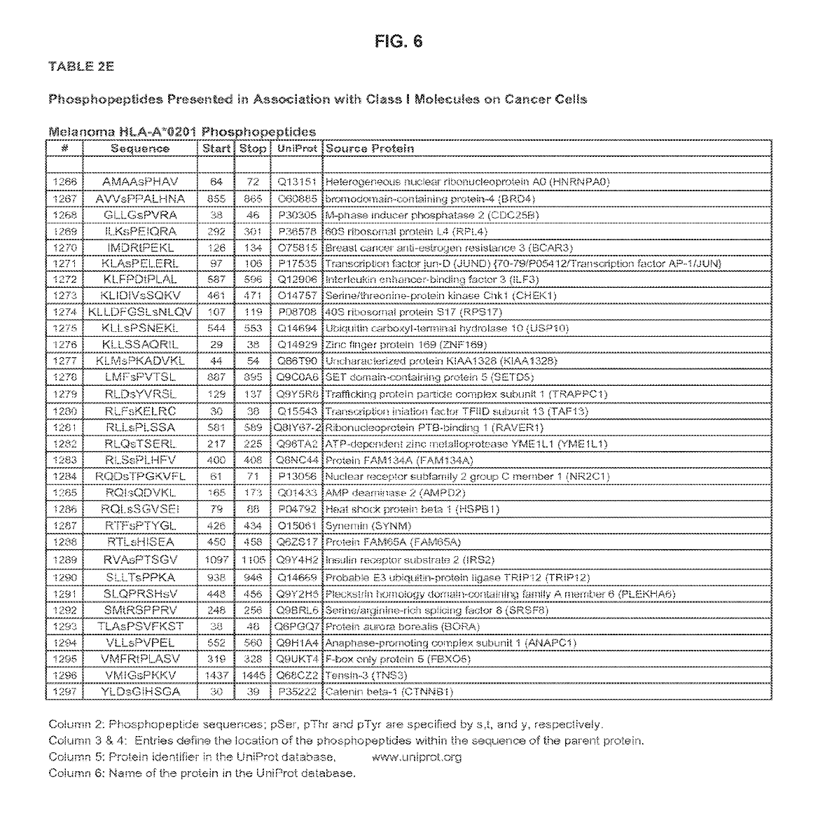

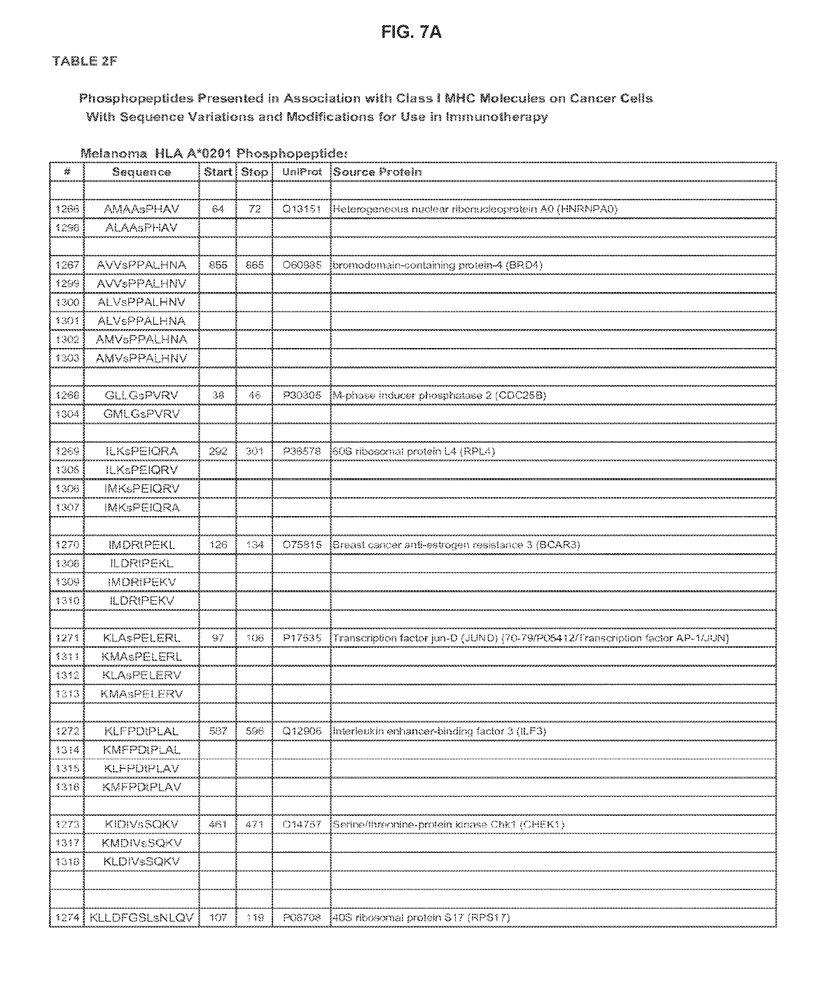

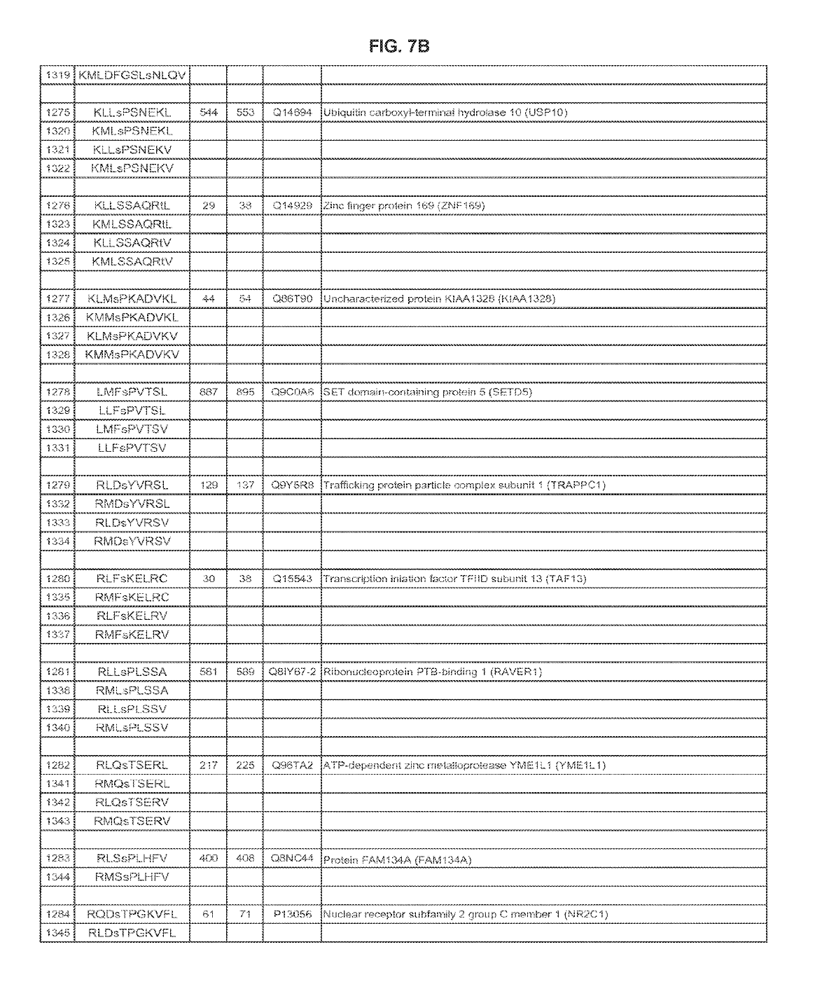

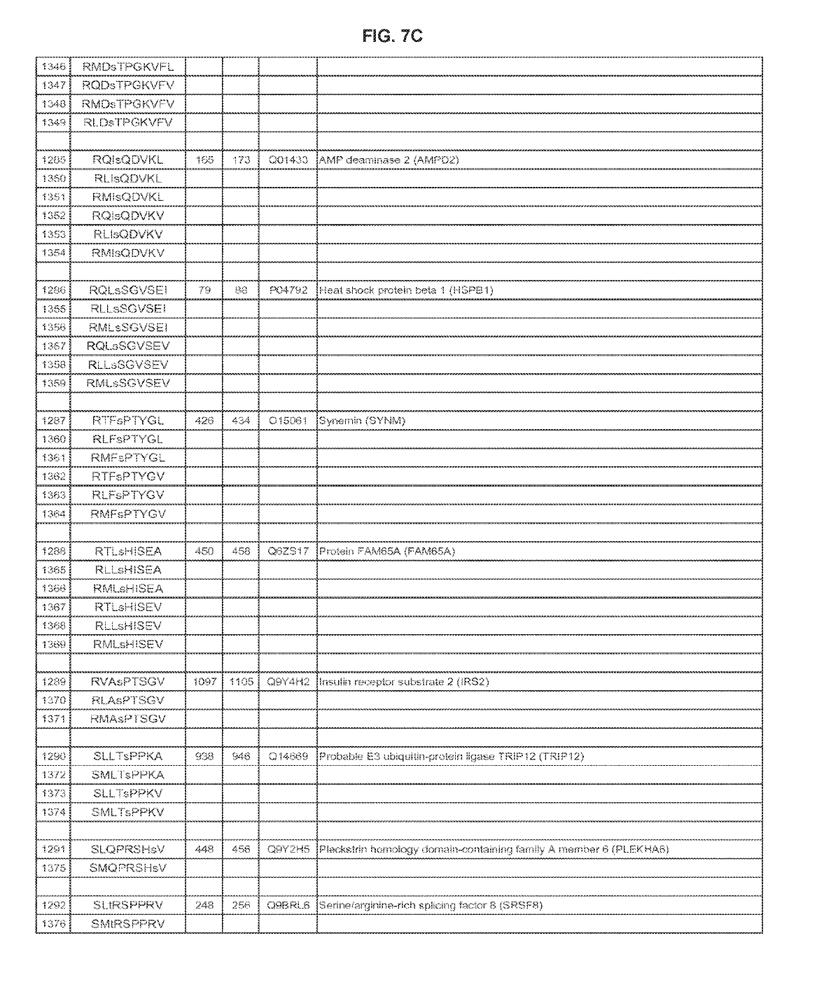

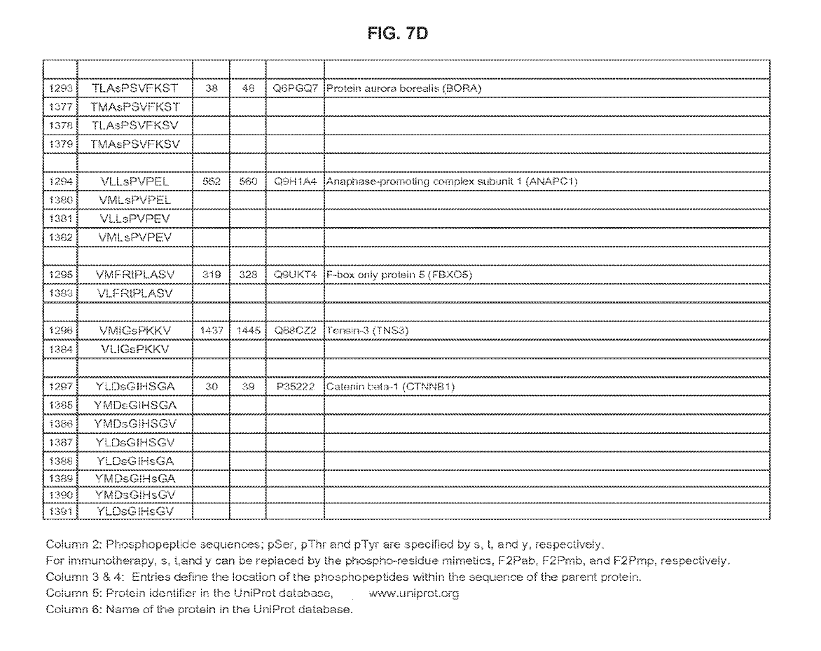

Tables 2A through 2F are shown in FIGS. 2A-7D. Sequence identifiers are listed in the first column. UniProt database sequences provide the sequences of the full human proteins from which the peptides are derived. The UniProt sequences are incorporated by reference.

The class I phosphopeptide antigens reported here allow adoptive T-cell therapy to be extended to melanoma patients that do not express the HLA-A*0201 allele and also make it possible to treat a variety of other cancers by the same approach.

We have also shown that we can clone the T-cell receptor on the murine cytotoxic T-cells and then inject the corresponding DNA into normal human T-cells. This process turns them into cytotoxic T-cells that now recognize cancer cells that express the same class I phosphopeptides derived from IRS-2 and β-catenin. In short, we have now demonstrated that this process can be used to convert cancer patient T- cells into activated cytotoxic T-cell that recognize class I phosphopeptides and kill their tumor. These experiments also open the door for using class I phosphopeptides in adopted T-cell therapy of cancer. This approach has shown dramatic success in the treatment of advanced stage metastatic melanoma. In conclusion, it should be noted that HLA A*0201 and HLA *A0301 both present peptides from the IRS-2 protein that contain the same phosphorylation site, Ser1100. RVApSPTSGV (SEQ ID NO: 1289) binds to HLA A*0201 and both RVApSPTSGVK (SEQ ID NO: 53) and RVApSPTSGVKR (SEQ ID NO: 54) bind to HLA A*0301. Neither of the A*0301 peptides bind to A*0201 and the A*0201 peptide cannot be presented by the A*0301 molecule.

### REFERENCES

The disclosure of each reference cited is expressly incorporated herein.

1. The Cancer Vaccine Roller Coaster, Goldman B and DeFrancesco, L, Nature Biotech, 2009, 27, 129

[symbol]

2. Phosphopeptide Antigens Associated with MHC Molecules, US 2005/0277161 Al, Dec. 15, 2005

3. Phosphorylated Peptides are Naturally Processed and Presented by Major Hitocompatibility Complex Class I Molecules In Vivo, Zarling A L, Ficarro S B, White F M, Shabanowitz J, Hunt D F, and Engelhard V H, J. Exp. Med., 2000, 192, 1755-1762.

4. Identification of Class I MHC-Associated Phosphopeptides as Targets for Cancer Immunotherapy, Zarling A L, Polefrone J M, Evans A M, Mikesh L M, Shahan ewis S T, Engelhard V H, and Hunt D F, Proc Natl Acad Sci, USA, 2006, 103, 14889-14894.

5. Phosphorylation-Dependent Interaction between Antigenic Peptides and MHC Class I: A Molecular Basis for the Presentation of Transformed Self, Mohammed F, Cobbold M, Zarling A L, Salim M, Barrett-Wilt G A, Shabanowitz J, Hunt D F, Engelhard V E, Willcox B E, Nat. Immunol. 2008, 11,1236-43.

6. Adoptive Cell Therapy for Patients with Metastatic Melanoma: Evaluation of Intensive Myeloablative Chemoradiation Preparative Regimens, Dudley M E, Yang J C, Sherry R, Hughes M S, Royal R, Kammula U, Robbins P F, Huang J P, Citrin D E, Leitman S F, Wunderlich J, Restifo N P, Tomasian A, Downey S G, Smith F O, Klaper J, Morton K, Laurencot C, White D E and Rosenberg S A, J. Clin Oncology, 2008, 26, 5233-5239.

7. Adoptive Cell Therapy for the Treatment of Patients with Metastatic Melanoma, Rosenberg S A and Dudley M E, Curr Opinion in Immun, 2009, 21, 233-240

1. A composition comprising one or more recombinant or synthetic peptides, wherein each recombinant or synthetic peptide is 8 to 50 amino acid residues long, and comprises an amino acid sequence selected from the group consisting of SEQ ID NOs: 1-1391.

2.-7. (canceled)

8. The composition of claim 1, further comprising an ajuvant, a pH stabilizing agent, a wetting agent, or an emulsifying agent.

9.-26. (canceled)

27. An antibody that specifically binds to a recombinant or synthetic peptide comprising an amino acid sequence selected from the group consisting of SEQ ID NOs: 1-1391.

28.-50. (canceled)

51. The composition of claim 1, which comprises an adjuvant.

52. The composition of claim 51, wherein the adjuvant is selected from the group consisting of complete Freund's adjuvant, incomplete Freund's adjuvant, aluminum hydroxide, lysolecithin, pluronic polyols, dinitrophenol, bacille Calmette-Guerin (BCG), and corynebacterium parvum, or combinations thereof.

53.1 The composition of claim 51, wherein the composition stimulates a T cell mediated immune response to at least one of the synthetic peptides when administered to a subject.

54. The composition of claim 1, wherein at least one of the recombinant or synthetic peptides is a phosphopeptide or a phosphopeptide mimetic.

55. The composition of claim 54, wherein the phosphopeptide mimetic comprises a mimetic of phosphoserine, phosphothreonine, or phosphotyrosine.

56. The composition of claim 55, wherein the mimetic of phosphoserine, phosphothreonine, or phosphotyrosine comprises a phosphorous atom is linked to the serine, threonine, or tyrosine amino acid residue through a carbon atom.

57. The composition of claim 56, wherein the mimetic of phosphoserine, phosphothreonine, or phosphotyrosine comprises a —CF₂—PO₃H group.

58. The composition of claim 56, wherein the mimetic of phosphoserine, phosphothreonine, or phosphotyrosine comprises a —CH₂—PO₃H group.

59. An in vitro composition comprising dendritic cells loaded with one or more recombinant or synthetic peptides, wherein each recombinant or synthetic peptide:
(i) is 8 to 50 amino acids long; and
(ii) comprises an amino acid sequence selected from the group consisting of SEQ ID NOs: 1-1391.
